SEQUENCE LISTING (196 sequences)

SEQ ID NO: 1 - DNA, 21 bp, Homo sapiens
tcatgagtggcagctgcaatt

SEQ ID NO: 2 - DNA, 19 bp, Homo sapiens
cagtctgatcagaactgtc

SEQ ID NO: 3 - DNA, 20 bp, Homo sapiens
catctcctagcatacgaatg

SEQ ID NO: 4 - DNA, 19 bp, Homo sapiens
ggctccttacacgtacatc

SEQ ID NO: 5 - DNA, 20 bp, Homo sapiens
gacagacacttcaatttcag

SEQ ID NO: 6 - DNA, 19 bp, Homo sapiens
gctggaaacttagtagcct

SEQ ID NO: 7 - DNA, 18 bp, Homo sapiens
gtgaagatctaagatatc

SEQ ID NO: 8 - DNA, 19 bp, Homo sapiens
tgaaaccgcagtaccacga

SEQ ID NO: 9 - DNA, 19 bp, Homo sapiens
aggttaagcttactagact

SEQ ID NO: 10 - DNA, 19 bp, Homo sapiens
tggaagtactcaattaatc

SEQ ID NO: 11 - DNA, 20 bp, Homo sapiens
ctacgaagtttcatcatatc

SEQ ID NO: 12 - DNA, 19 bp, Homo sapiens
ggactgcgttatggtaagg

SEQ ID NO: 13 - DNA, 19 bp, Homo sapiens
catggcctgagtaacatcc

SEQ ID NO: 14 - DNA, 19 bp, Homo sapiens
cagaattggcaagcttagg

SEQ ID NO: 15 - DNA, 19 bp, Homo sapiens
atgatccagatcatgagtg

SEQ ID NO: 16 - DNA, 19 bp, Homo sapiens
gttgccaagtaatggttag

SEQ ID NO: 17 - DNA, 19 bp, Homo sapiens
cagtactgaggtattcttg

SEQ ID NO: 18 - DNA, 18 bp, Homo sapiens
cactgcagatctccagtg

SEQ ID NO: 19 - DNA, 19 bp, Homo sapiens
gaataagcctgattctctc

SEQ ID NO: 20 - DNA, 20 bp, Homo sapiens
gagcacctatctgtgtctcc

SEQ ID NO: 21 - DNA, 20 bp, Homo sapiens
gtggctagtatcagagcacc

SEQ ID NO: 22 - DNA, 19 bp, Homo sapiens
gactgatgtctgtggctag

SEQ ID NO: 23 - DNA, 19 bp, Homo sapiens
gtgtctggtgtcttctagg

SEQ ID NO: 24 - DNA, 20 bp, Homo sapiens
ccacgtccgacatgggttac

SEQ ID NO: 25 - DNA, 19 bp, Homo sapiens
tctatatctccttgatgga

SEQ ID NO: 26 - DNA, 19 bp, Homo sapiens
tagattcagtgtgacgatc

SEQ ID NO: 27 - DNA, 19 bp, Homo sapiens
accaatctggatgttggca

SEQ ID NO: 28 - DNA, 19 bp, Homo sapiens
ctggatgatccagatcatg

SEQ ID NO: 29 - DNA, 18 bp, Homo sapiens
ggcttgtccattataatc

SEQ ID NO: 30 - DNA, 20 bp, Homo sapiens
acatctcctagcatacgaat

SEQ ID NO: 31 - DNA, 21 bp, Homo sapiens
aaggttaagcttactagacta

SEQ ID NO: 32 - DNA, 21 bp, Homo sapiens
gtgcatggcctgagtaacatc

SEQ ID NO: 33 - DNA, 21 bp, Homo sapiens
  misc_feature: All linkages are phosphorothioate
acagtgcatggcctgagtaac

SEQ ID NO: 34 - DNA, 21 bp, Homo sapiens
cagaattggcaagcttaggtt

SEQ ID NO: 35 - DNA, 21 bp, Homo sapiens
tggcaagcttaggttccttta

SEQ ID NO: 36 - DNA, 20 bp, Homo sapiens
tgatccagatcatgagtggc

SEQ ID NO: 37 - DNA, 21 bp, Homo sapiens
taaagttgccaagtaatggtt

SEQ ID NO: 38 - DNA, 21 bp, Homo sapiens
actgaggtattcttgtataaa

SEQ ID NO: 39 - DNA, 20 bp, Homo sapiens
gcagatctccagtggtgatt

SEQ ID NO: 40 - DNA, 21 bp, Homo sapiens
catacacttggcttgtccatt

SEQ ID NO: 41 - DNA, 21 bp, Homo sapiens
  misc_feature: All linkages are phosphorothioate
taagtaacagtgcatggcctg

SEQ ID NO: 42 - DNA, 19 bp, Homo sapiens
  misc_feature: All linkages are phosphorothioate
ggttgctcagntctgcaca

SEQ ID NO: 43 - DNA, 16 bp, Homo sapiens
  misc_feature (2)..(12): Nucleotides 2 and 12 are inosine
ancatccttttnaact

SEQ ID NO: 44 - DNA, 16 bp, Homo sapiens
  misc_feature (3)..(3): Nucleotide 3 is inosine
agnccccatttgttca

SEQ ID NO: 45 - DNA, 17 bp, Homo sapiens
  misc_feature (6)..(12): Nucleotides 6 and 12 are inosine
gccacntcagcntggta

SEQ ID NO: 46 - DNA, 17 bp, Homo sapiens
  misc_feature (9)..(12): Nucleotides 9 and 12 are inosine
accatgtcnatnaccat

SEQ ID NO: 47 - DNA, 16 bp, Homo sapiens
  misc_feature (5)..(11): Nucleotides 5 and 11 are inosine
tcatntgtttngacat

SEQ ID NO: 48 - DNA, 17 bp, Homo sapiens
  misc_feature (3)..(6): Nucleotides 3 and 6 are inosine
ganctngtcactttctt

SEQ ID NO: 49 - DNA, 17 bp, Homo sapiens
  misc_feature (15)..(15): Nucleotide 15 is inosine
gcacagtgcaccatntt

SEQ ID NO: 50 - DNA, 18 bp, Homo sapiens
  misc_feature (7)..(10): Nucleotides 7 and 10 are inosine
gctcagntcngcacagtg

SEQ ID NO: 51 - DNA, 17 bp, Homo sapiens
  misc_feature (9)..(15): Nucleotides 9 and 15 are inosine
tcacacatngggctnat

SEQ ID NO: 52 - DNA, 17 bp, Homo sapiens
  misc_feature (3)..(12): Nucleotides 3 and 12 are inosine
ttntcacacatngggct

SEQ ID NO: 53 - DNA, 17 bp, Homo sapiens
  misc_feature (7)..(7): Nucleotide 7 is inosine
gccattntccacatcaa

SEQ ID NO: 54 - DNA, 17 bp, Homo sapiens
  misc_feature (8)..(9): Nucleotides 8 and 9 are inosine
ggatccannggactccg

SEQ ID NO: 55 - DNA, 20 bp, Homo sapiens
  misc_feature (7)..(16): Nucleotides 7 and 16 are inosine
gtcatantcgctgtcngatc

SEQ ID NO: 56 - DNA, 18 bp, Homo sapiens
  misc_feature (7)..(13): Nucleotides 7 and 13 are inosine
gacctgngcaaanggagt

SEQ ID NO: 57 - DNA, 17 bp, Homo sapiens
  misc_feature (15)..(15): Nucleotide 15 is inosine
gccatctcactgacnga

SEQ ID NO: 58 - DNA, 17 bp, Homo sapiens
  misc_feature (6)..(15): Nucleotides 6 and 15 are inosine
cttttnaacttgttnga

SEQ ID NO: 59 - DNA, 17 bp, Homo sapiens
  misc_feature (9)..(12): Nucleotides 9 and 12 are inosine
tctaagaangtntttga

SEQ ID NO: 60 - DNA, 17 bp, Homo sapiens
  misc_feature (6)..(15): Nucleotides 6 and 15 are inosine
tgcttntctaagaangt

SEQ ID NO: 61 - DNA, 17 bp, Homo sapiens
  misc_feature (7)..(10): Nucleotides 7 and 10 are inosine
ttcagtnttnactccaa

SEQ ID NO: 62 - DNA, 17 bp, Homo sapiens
  misc_feature (9)..(11): Nucleotides 9 and 11 are inosine
tggtcttcnanagtcat

SEQ ID NO: 63 - DNA, 17 bp, Homo sapiens
  misc_feature (15)..(15): Nucleotide 15 is inosine
atctccaaatctgtnaa

SEQ ID NO: 64 - DNA, 17 bp, Homo sapiens
  misc_feature (3)..(3): Nucleotide 3 is inosine
agnatctccaaatctgt

SEQ ID NO: 65 - DNA, 17 bp, Homo sapiens
  misc_feature (9)..(12): Nucleotides 9 and 12 are inosine
ggatgatcnacntcatg

SEQ ID NO: 66 - DNA, 17 bp, Homo sapiens
  misc_feature (3)..(15): Nucleotides 3 and 15 are inosine
acnccaggatgatcnac

SEQ ID NO: 67 - DNA, 17 bp, Homo sapiens
  misc_feature (9)..(12): Nucleotides 9 and 12 are inosine
gcaagttcngantttgt

SEQ ID NO: 68 - DNA, 18 bp, Homo sapiens
  misc_feature (4)..(7): Nucleotides 4 and 7 are inosine
cttngtngggttgctcag

SEQ ID NO: 69 - DNA, 17 bp, Homo sapiens
  misc_feature (9)..(15): Nucleotides 9 and 15 are inosine
tcacacatngggctnat

SEQ ID NO: 70 - DNA, 17 bp, Homo sapiens
  misc_feature (6)..(12): Nucleotides 6 and 12 are inosine
ggatgnacaatntagtc

SEQ ID NO: 71 - DNA, 17 bp, Homo sapiens
  misc_feature (3)..(3): Nucleotide 3 is inosine
gangccatctcactgac

SEQ ID NO: 72 - DNA, 17 bp, Homo sapiens
  misc_feature (13)..(15): Nucleotides 13 and 15 are inosine
gtaatggtcttcnanag

SEQ ID NO: 73 - DNA, 21 bp, Homo sapiens
  misc_feature: All linkages are phosphorothioate
tcatgagtggcagctgcaatt

SEQ ID NO: 74 - DNA, 21 bp, Homo sapiens
  misc_feature: All linkages are phosphorothioate
tcntgngtggcngctgcnntt

SEQ ID NO: 75 - DNA, 21 bp, Homo sapiens
tcatgagtggcagctgcaatt

SEQ ID NO: 76 - DNA, 21 bp, Homo sapiens
  misc_feature: Nucleotides3, 6, 12, 18-19 are 2-amino-2′deoxyadenosine
tcntgngtggcngctgcnntt

SEQ ID NO: 77 - DNA, 21 bp, Homo sapiens
  misc_feature (1)..(5): Nucleotides are 2′-OMe-RNA
tcatgagtggcagctgcaatt

SEQ ID NO: 78 - DNA, 21 bp, Homo sapiens
  misc_feature (1)..(9): Linkage to next nucleotide is phosphorothioate
tcatgagtggcagctgcaatt

SEQ ID NO: 79 - DNA, 21 bp, Homo sapiens
  misc_feature (1)..(7): Linkage to next nucleotide is phosphorothioate
tcatgagtggcagctgcaatt

SEQ ID NO: 80 - DNA, 21 bp, Homo sapiens
  misc_feature (1)..(5): Linkage to next nucleotide is phosphorothioate
tcatgagtggcagctgcaatt

SEQ ID NO: 81 - DNA, 21 bp, Homo sapiens
  misc_feature: Nucleotides 6, 12, 18-19 are 2-amino-2′deoxyadenosine
tcntgngtggcngctgcnntt

SEQ ID NO: 82 - DNA, 21 bp, Homo sapiens
  misc_feature (1)..(4): Linkage to next nucleotide is phosphorothioate
tcatgagtggcagctgcaatt

SEQ ID NO: 83 - DNA, 21 bp, Homo sapiens
  misc_feature (1)..(4): Linkage to next nucleotide is phosphorothioate
tcatgagtggcagctgcaatt

SEQ ID NO: 84 - DNA, 21 bp, Homo sapiens
  misc_feature (1)..(4): Linkage to next nucleotide is phosphorothioate
tcatgagtggcagctgcaatt

SEQ ID NO: 85 - DNA, 21 bp, Homo sapiens
  misc_feature (1)..(2): Linkage to next nucleotide is phosphorothioate
tcatgagtggcagctgcaatt

SEQ ID NO: 86 - DNA, 21 bp, Homo sapiens
  misc_feature: Nucleotides 3, 6, 12, 18-19 are 2-amino-2′deoxyadenosine
tcntgngtggcngctgcnntt

SEQ ID NO: 87 - DNA, 21 bp, Homo sapiens
  misc_feature (1)..(2): Linkage to next nucleotide is phosphorothioate
tcatgagtggcagctgcaatt

SEQ ID NO: 88 - DNA, 21 bp, Homo sapiens
  misc_feature: Nucleotides 3, 6, 12, 18-19 are 2-amino-2′deoxyadenosine
tcntgngtggcngctgcnntt

SEQ ID NO: 89 - DNA, 21 bp, Homo sapiens
  misc_feature (1)..(2): Linkage to next nucleotide is phosphorothioate
tcatgagtggcagctgcaatt

SEQ ID NO: 90 - DNA, 21 bp, Homo sapiens
  misc_feature: Nucleotides 3, 6, 12, 18-19 are 2-amino-2′deoxyadenosine
tcntgngtggcngctgcnntt

SEQ ID NO: 91 - DNA, 21 bp, Homo sapiens
  misc_feature (1)..(2): Linkage to next nucleotide is phosphorothioate
tcatgagtggcagctgcaatt

SEQ ID NO: 92 - DNA, 21 bp, Homo sapiens
  misc_feature: Nucleotides 3, 6, 12, 18-19 are 2-amino-2′deoxyadenosine
tcntgngtggcngctgcnntt

SEQ ID NO: 93 - DNA, 20 bp, Homo sapiens
  misc_feature: All linkages are phosphorothioate
tcatggtggcagctgcaatt

SEQ ID NO: 94 - DNA, 21 bp, Homo sapiens
  misc_feature (1)..(2): Linkage to next nucleotide is phosphorothioate
acagtgcatggcctgagtaac

SEQ ID NO: 95 - DNA, 21 bp, Homo sapiens
  misc_feature: Nucleotides 1, 3, 8, 16, 19-20 are 2-amino-2′deoxyadenosine
ncngtgcntggcctgngtnnc

SEQ ID NO: 96 - DNA, 21 bp, Homo sapiens
  misc_feature (1)..(2): Linkage to next nucleotide is phosphorothioate
acagtgcatggcctgagtaac

SEQ ID NO: 97 - DNA, 21 bp, Homo sapiens
  misc_feature: Nucleotides 1, 3, 8, 16, 19-20 are 2-amino-2′deoxyadenosine
ncngtgcntggcctgngtnnc

SEQ ID NO: 98 - DNA, 21 bp, Homo sapiens
  misc_feature (1)..(2): Linkage to next nucleotide is phosphorothioate
acagtgcatggcctgagtaac

SEQ ID NO: 99 - DNA, 21 bp, Homo sapiens
  misc_feature: Nucleotides 1, 3, 8, 16, 19-20 are 2-amino-2′deoxyadenosine
ncngtgcntggcctgngtnnc

SEQ ID NO: 100 - DNA, 21 bp, Homo sapiens
  misc_feature (1)..(2): Linkage to next nucleotide is phosphorothioate
taagtaacagtgcatggcctg

SEQ ID NO: 101 - DNA, 21 bp, Homo sapiens
  misc_feature: Nucleotides 2, 3, 6, 7, 9, 14 are 2-amino-2′deoxyadenosine
tnngtnncngtgcntggcctg

SEQ ID NO: 102 - DNA, 21 bp, Homo sapiens
  misc_feature (1)..(2): Linkage to next nucleotide is phosphorothioate
taagtaacagtgcatggcctg

SEQ ID NO: 103 - DNA, 21 bp, Homo sapiens
  misc_feature: Nucleotides 2, 3, 6, 7, 9, 14 are 2-amino-2′deoxyadenosine
tnngtnncngtgcntggcctg

SEQ ID NO: 104 - DNA, 21 bp, Homo sapiens
  misc_feature (1)..(2): Linkage to next nucleotide is phosphorothioate
taagtaacagtgcatggcctg

SEQ ID NO: 105 - DNA, 21 bp, Homo sapiens
  misc_feature: Nucleotides 2, 3, 6, 7, 9, 14 are 2-amino-2′deoxyadenosine
tnngtnncngtgcntggcctg

SEQ ID NO: 106 - DNA, 19 bp, Homo sapiens
  misc_feature: All linkages are phosphorothioate
ggttgctcagntctgcaca

SEQ ID NO: 107 - DNA, 19 bp, Homo sapiens
  misc_feature: All linkages are phosphorothioate
ggttgctcngntctgcncn

SEQ ID NO: 108 - DNA, 19 bp, Homo sapiens
  misc_feature (11)..(11): Nucleotide 11 is inosine
ggttgctcagntctgcaca

SEQ ID NO: 109 - DNA, 19 bp, Homo sapiens
  misc_feature: Nucleotides 9, 17, 19 are 2-amino-2′deoxyadenosine
ggttgctcngntctgcncn

SEQ ID NO: 110 - DNA, 19 bp, Homo sapiens
  misc_feature (1)..(5): Nucleotides are 2′-OMe-RNA
ggttgctcagntctgcaca

SEQ ID NO: 111 - DNA, 19 bp, Homo sapiens
  misc_feature (1)..(8): Linkage to next nucleotide is phosphorothioate
ggttgctcagntctgcaca

SEQ ID NO: 112 - DNA, 19 bp, Homo sapiens
  misc_feature (1)..(6): Linkage to next nucleotide is phosphorothioate
ggttgctcagntctgcaca

SEQ ID NO: 113 - DNA, 19 bp, Homo sapiens
  misc_feature (1)..(5): Linkage to next nucleotide is phosphorothioate
ggttgctcagntctgcaca

SEQ ID NO: 114 - DNA, 19 bp, Homo sapiens
  misc_feature: Nucleotides 9, 17, 19 are 2-amino-2′deoxyadenosine
ggttgctcngntctgcncn

SEQ ID NO: 115 - DNA, 19 bp, Homo sapiens
  misc_feature (1)..(4): Linkage to next nucleotide is phosphorothioate
ggttgctcagntctgcaca

SEQ ID NO: 116 - DNA, 19 bp, Homo sapiens
  misc_feature (1)..(4): Linkage to next nucleotide is phosphorothioate
ggttgctcagntctgcaca

SEQ ID NO: 117 - DNA, 19 bp, Homo sapiens
  misc_feature (1)..(4): Linkage to next nucleotide is phosphorothioate
ggttgctcagntctgcaca

SEQ ID NO: 118 - DNA, 19 bp, Homo sapiens
  misc_feature (1)..(2): Linkage to next nucleotide is phosphorothioate
ggttgctcagntctgcaca

SEQ ID NO: 119 - DNA, 19 bp, Homo sapiens
  misc_feature: Nucleotides 9, 17, 19 are 2-amino-2′deoxyadenosine
ggttgctcngntctgcncn

SEQ ID NO: 120 - DNA, 19 bp, Homo sapiens
  misc_feature (1)..(2): Linkage to next nucleotide is phosphorothioate
ggttgctcagntctgcaca

SEQ ID NO: 121 - DNA, 19 bp, Homo sapiens
  misc_feature: Nucleotides 9, 17, 19 are 2-amino-2′deoxyadenosine
ggttgctcngntctgcncn

SEQ ID NO: 122 - DNA, 19 bp, Homo sapiens
  misc_feature (1)..(2): Linkage to next nucleotide is phosphorothioate
ggttgctcagntctgcaca

SEQ ID NO: 123 - DNA, 19 bp, Homo sapiens
  misc_feature: Nucleotides 9, 17, 19 are 2-amino-2′deoxyadenosine
ggttgctcngntctgcncn

SEQ ID NO: 124 - DNA, 19 bp, Homo sapiens
  misc_feature (1)..(3): Linkage to next nucleotide is phosphorothioate
ggttgctcagntctgcaca

SEQ ID NO: 125 - DNA, 19 bp, Homo sapiens
  misc_feature: Nucleotides 9, 17, 19 are 2-amino-2′deoxyadenosine
ggttgctcngntctgcncn

SEQ ID NO: 126 - DNA, 19 bp, Homo sapiens
  misc_feature (1)..(2): Linkage to next nucleotide is phosphorothioate
ggttgctcagntctgcaca

SEQ ID NO: 127 - DNA, 19 bp, Homo sapiens
  misc_feature: Nucleotides 9, 17, 19 are 2-amino-2′deoxyadenosine
ggttgctcngntctgcncn

SEQ ID NO: 128 - DNA, 21 bp, Homo sapiens
  misc_feature: All linkages are phosphorothioate
agtactcaccgtcgacgttaa

SEQ ID NO: 129 - DNA, 21 bp, Homo sapiens
  misc_feature: All linkages are phosphorothioate
tgtcacgtaccggactcattg

SEQ ID NO: 130 - DNA, 21 bp, Homo sapiens
  misc_feature: All linkages are phosphorothioate
attcattgtcacgtaccggac

SEQ ID NO: 131 - DNA, 19 bp, Homo sapiens
  misc_feature: All linkages are phosphorothioate
ccaacgagtcnagacgtgt

SEQ ID NO: 132 - DNA, 21 bp, Homo sapiens
  misc_feature: Nucleotides 1, 5, 11, 15, 20 are 2-amino-2′deoxyadenosine
nttcnttgtcncgtnccggnc

SEQ ID NO: 133 - DNA, 20 bp, Homo sapiens
  misc_feature (1)..(2): Linkage to next nucleotide is phosphorothioate
gggggacgatcgtcgggggg

SEQ ID NO: 134 - DNA, 17 bp, Mus musculus
  misc_feature: All linkages are phosphorothioate
tccagatcgtgagtggc

SEQ ID NO: 135 - DNA, 17 bp, Mus musculus
  misc_feature: All linkages are phosphorothioate
tgctcaacgtctgaggg

SEQ ID NO: 136 - DNA, 17 bp, Mus musculus
  misc_feature: Nucleotides 4, 6, 12 are 2-amino-2′deoxyadenosine
tccngntcgtgngtggc

SEQ ID NO: 137 - DNA, 20 bp, Mus musculus
  misc_feature: All linkages are phosphorothioate
ggttgctcaggtctgcacag

SEQ ID NO: 138 - DNA, 20 bp, Mus musculus
  misc_feature: Nucleotides 9, 17, 19 are 2-amino-2′deoxyadenosine
ggttgctcnggtctgcncng

SEQ ID NO: 139 - DNA, 20 bp, Homo sapiens
tcaggccatgcactgttact

SEQ ID NO: 140 - DNA, 21 bp, Homo sapiens
cctgattctctcaataagccc

SEQ ID NO: 141 - DNA, 20 bp, Homo sapiens
tggcagacctgaagacaatg

SEQ ID NO: 142 - DNA, 19 bp, Homo sapiens
aaattcctccatgatgcgg

SEQ ID NO: 143 - DNA, 20 bp, Homo sapiens
cagaatatggtgcactgtgc

SEQ ID NO: 144 - DNA, 21 bp, Homo sapiens
agtctatgaagcccacctgtg

SEQ ID NO: 145 - DNA, 20 bp, Homo sapiens
caataggaagatctcagtgc

SEQ ID NO: 146 - DNA, 20 bp, Homo sapiens
gtgttcaagtcttcggagtt

SEQ ID NO: 147 - DNA, 20 bp, Homo sapiens
agggcacatcctatgacagc

SEQ ID NO: 148 - DNA, 20 bp, Homo sapiens
atttgttgcccaggaaagtg

SEQ ID NO: 149 - DNA, 28 bp, Homo sapiens
ccagtgacaaaaatgaaatatagtggaa

SEQ ID NO: 150 - DNA, 20 bp, Homo sapiens
gactccacatcgaggtgcgt

SEQ ID NO: 151 - DNA, 21 bp, Homo sapiens
cagggacaggatatggagcaa

SEQ ID NO: 152 - DNA, 24 bp, Homo sapiens
gcagactcaaattccagcttgtta

SEQ ID NO: 153 - DNA, 26 bp, Homo sapiens
caaggactggtctttctatctcttgt

SEQ ID NO: 154 - DNA, 22 bp, Homo sapiens
gtggagcaacctgctcagatac

SEQ ID NO: 155 - DNA, 21 bp, Homo sapiens
  misc_feature: Nucleotides 1, 4, 8, 15, 20, 21 are 2-amino-2′deoxyadenosine
ngtnctcnccgtcgncgttnn

SEQ ID NO: 156 - DNA, 19 bp, Homo sapiens
  misc_feature: Nucleotides 3, 4, 7, 12, 14 are 2-amino-2′deoxyadenosine
ccnncgngtcnngncgtgt

SEQ ID NO: 157 - DNA, 19 bp, Homo sapiens
  misc_feature: All linkages are phosphorothioate
gggtctgcngcgggntggt

SEQ ID NO: 158 - DNA, 21 bp, Homo sapiens
  misc_feature: All linkages are phosphorothioate
gttnctncttccncctgcctg

SEQ ID NO: 159 - DNA, 18 bp, Rattus norvegicus
  misc_feature: All linkages are phosphorothioate
tggcactttaggtggctg

SEQ ID NO: 160 - DNA, 18 bp, Rattus norvegicus
  misc_feature: All linkages are phosphorothioate
actcatattcatagggtg

SEQ ID NO: 161 - DNA, 19 bp, Homo sapiens
  misc_feature: All linkages are phosphorothioate
cacctctgtcaccagcatg

SEQ ID NO: 162 - DNA, 19 bp, Homo sapiens
  misc_feature: Nucleotides 2, 11, 14, 17 are 2-amino-2′deoxyadenosine
cncctctgtcnccngcntg

SEQ ID NO: 163 - DNA, 19 bp, Homo sapiens
  misc_feature: Nucleotides 2, 11, 14, 17 are 2-amino-2′deoxyadenosine
cncctctgtcnccngcntg

SEQ ID NO: 164 - DNA, 19 bp, Homo sapiens
  misc_feature: Nucleotides 2, 11, 14, 17 are 2-amino-2′deoxyadenosine
cncctctgtcnccngcntg

SEQ ID NO: 165 - DNA, 19 bp, Homo sapiens
  misc_feature: Nucleotides 2, 11, 14, 17 are 2-amino-2′deoxyadenosine
cncctctgtcnccngcntg

SEQ ID NO: 166 - DNA, 19 bp, Homo sapiens
  misc_feature: Nucleotides 2, 11, 14, 17 are 2-amino-2′deoxyadenosine
cncctctgtcnccngcntg

SEQ ID NO: 167 - DNA, 19 bp, Homo sapiens
  misc_feature: Nucleotides 2, 11, 14, 17 are 2-amino-2′deoxyadenosine
cncctctgtcnccngcntg

SEQ ID NO: 168 - DNA, 19 bp, Homo sapiens
  misc_feature: Nucleotides 2, 11, 14, 17 are 2-amino-2′deoxyadenosine
cncctctgtcnccngcntg

SEQ ID NO: 169 - DNA, 19 bp, Homo sapiens
  misc_feature: Nucleotides 2, 11, 14, 17 are 2-amino-2′deoxyadenosine
cncctctgtcnccngcntg

SEQ ID NO: 170 - DNA, 19 bp, Homo sapiens
  misc_feature: Nucleotides 2, 11, 14, 17 are 2-amino-2′deoxyadenosine
cncctctgtcnccngcntg

SEQ ID NO: 171 - DNA, 19 bp, Homo sapiens
  misc_feature: Nucleotides 2, 11, 14, 17 are 2-amino-2′deoxyadenosine
cncctctgtcnccngcntg

SEQ ID NO: 172 - DNA, 21 bp, Homo sapiens
  misc_feature: All linkages are phosphorothioate
gtacttccggaacctctctcc

SEQ ID NO: 173 - DNA, 21 bp, Homo sapiens
  misc_feature: Nucleotides 3, 11, 12 are 2-amino-2′deoxyadenosine
gtncttccggnncctctctcc

SEQ ID NO: 174 - DNA, 21 bp, Homo sapiens
  misc_feature: Nucleotides 3, 11, 12 are 2-amino-2′deoxyadenosine
gtncttccggnncctctctcc

SEQ ID NO: 175 - DNA, 21 bp, Homo sapiens
  misc_feature: Nucleotides 3, 11, 12 are 2-amino-2′deoxyadenosine
gtncttccggnncctctctcc

SEQ ID NO: 176 - DNA, 21 bp, Homo sapiens
  misc_feature: Nucleotides 3, 11, 12 are 2-amino-2′deoxyadenosine
gtncttccggnncctctctcc

SEQ ID NO: 177 - DNA, 21 bp, Homo sapiens
  misc_feature: Nucleotides 3, 11, 12 are 2-amino-2′deoxyadenosine
gtncttccggnncctctctcc

SEQ ID NO: 178 - DNA, 21 bp, Homo sapiens
  misc_feature: Nucleotides 3, 11, 12 are 2-amino-2′deoxyadenosine
gtncttccggnncctctctcc

SEQ ID NO: 179 - DNA, 20 bp, Homo sapiens
  misc_feature: All linkages are phosphorothioate
ctctccacttccacggcctg

SEQ ID NO: 180 - DNA, 20 bp, Homo sapiens
  misc_feature: Nucleotides 7, 13 are 2-amino-2′deoxyadenosine
ctctccncttccncggcctg

SEQ ID NO: 181 - DNA, 20 bp, Homo sapiens
  misc_feature: Nucleotides 7, 13 are 2-amino-2′deoxyadenosine
ctctccncttccncggcctg

SEQ ID NO: 182 - DNA, 20 bp, Homo sapiens
  misc_feature: Nucleotides 7, 13 are 2-amino-2′deoxyadenosine
ctctccncttccncggcctg

SEQ ID NO: 183 - DNA, 20 bp, Homo sapiens
  misc_feature: Nucleotides 7, 13 are 2-amino-2′deoxyadenosine
ctctccncttccncggcctg

SEQ ID NO: 184 - DNA, 20 bp, Homo sapiens
  misc_feature: Nucleotides 7, 13 are 2-amino-2′deoxyadenosine
ctctccncttccncggcctg

SEQ ID NO: 185 - DNA, 20 bp, Homo sapiens
  misc_feature: Nucleotides 7, 13 are 2-amino-2′deoxyadenosine
ctctccncttccncggcctg

SEQ ID NO: 186 - DNA, 20 bp, Homo sapiens
  misc_feature: Nucleotides 7, 13 are 2-amino-2′deoxyadenosine
ctctccncttccncggcctg

SEQ ID NO: 187 - DNA, 20 bp, Homo sapiens
  misc_feature: Nucleotides 7, 13 are 2-amino-2′deoxyadenosine
ctctccncttccncggcctg

SEQ ID NO: 188 - DNA, 20 bp, Homo sapiens
  misc_feature: Nucleotides 7, 13 are 2-amino-2′deoxyadenosine
ctctccncttccncggcctg

SEQ ID NO: 189 - DNA, 19 bp, Homo sapiens
  misc_feature: All linkages are phosphorothioate
gaccgagctggccacctcc

SEQ ID NO: 190 - DNA, 19 bp, Homo sapiens
  misc_feature: Nucleotides 2, 6, 14 are 2-amino-2′deoxyadenosine
gnccgngctggccncctcc

SEQ ID NO: 191 - DNA, 19 bp, Homo sapiens
  misc_feature: Nucleotides 2, 6, 14 are 2-amino-2′deoxyadenosine
gnccgngctggccncctcc

SEQ ID NO: 192 - DNA, 19 bp, Homo sapiens
  misc_feature: Nucleotides 2, 6, 14 are 2-amino-2′deoxyadenosine
gnccgngctggccncctcc

SEQ ID NO: 193 - DNA, 19 bp, Homo sapiens
  misc_feature: Nucleotides 2, 6, 14 are 2-amino-2′deoxyadenosine
gnccgngctggccncctcc

SEQ ID NO: 194 - DNA, 19 bp, Homo sapiens
  misc_feature: Nucleotides 2, 6, 14 are 2-amino-2′deoxyadenosine
gnccgngctggccncctcc

SEQ ID NO: 195 - DNA, 19 bp, Homo sapiens
  misc_feature: Nucleotides 2, 6, 14 are 2-amino-2′deoxyadenosine
gnccgngctggccncctcc

SEQ ID NO: 196 - DNA, 19 bp, Homo sapiens
  misc_feature: Nucleotides 2, 6, 14 are 2-amino-2′deoxyadenosine
gnccgngctggccncctcc

(19) United States
(12) Patent Application Publication
(10) Pub. No.: US 2014/0163085 A1
(43) Pub. Date: Jun. 12, 2014

(54) Oligonucleotide Inhibitors with Chimeric Backbone and 2-Amino-2'-Deoxyadenosine

(71) Applicants: Rosanne Seguin, Montreal (CA); Nicolay Ferrari, Boucherville (CA)

(72) Inventors: Rosanne Seguin, Montreal (CA); Nicolay Ferrari, Boucherville (CA)

(73) Assignee: PHARMAXIS LTD, New South Wales (AU)

(21) Appl. No.: 14/001,480

(22) PCT Filed: Feb. 23, 2012
(86) PCT No.: PCT/CA2012/000169

§ 371 Date: Feb. 12, 2014

Related U.S. Application Data
(60) Provisional application No. 61/446,144, filed on Feb. 24, 2011.

(51) Int. Cl. C12N 15/113

(52) U.S. Cl. CPC C12N 15/1137; C12N 15/1136; C12N 15/1138
USPC 514/44 A; 536/24.5

(57) ABSTRACT

There is provided herein, an oligonucleotide directed against a target gene, wherein the oligonucleotide is capable of hybridizing to at least a portion of a nucleic acid sequence encoding the gene under stringent conditions, and wherein, at least one nucleotide of the oligonucleotide is 2-amino-2′-deoxyadenosine (DAP); and the internucleoside linkages of the oligonucleotide comprises at least three alternating segments, each segment consisting of either at least one phosphorothioate or at least one phosphodiester bond.

### CROSS REFERENCES TO RELATED APPLICATIONS

This application claims priority from U.S. Provisional Patent Application No. 61/446,144 filed on Feb. 24, 2011.

### FIELD OF THE INVENTION

The present invention relates to oligonucleotides useful in the treatment of inflammation and respiratory diseases, and specifically to oligonucleotide inhibitors of phosphodiesterase expression.

### BACKGROUND OF THE INVENTION

The alveolar and airway epithelium is recognized as a dynamic barrier that plays an important role in regulating inflammatory and metabolic responses to oxidative stress, sepsis, endotoxemia, and other critical illnesses in the lung. The respiratory epithelium, in particular, is a primary target of inflammatory conditions/infections at the epithelial-blood interface, and is itself capable of amplifying an inflammatory signal by recruiting inflammatory cells and producing inflammatory mediators.

Chronic Obstructive Pulmonary Disease (COPD) is one example of an inflammatory airway and alveolar disease where persistent upregulation of inflammation is thought to play a role. Inflammation in COPD is characterized by increased infiltration of neutrophils, CD8 positive lymphocytes, and macrophages into the airways. Neutrophils and macrophages play an important role in the pathogenesis of airway inflammation in COPD because of their ability to release a number of mediators including elastase, metalloproteases, and oxygen radicals that promote tissue inflammation and damage. It has been suggested that inflammatory cell accumulation in the airways of patients with COPD is driven by increased release of pro-inflammatory cytokines and of chemokines that attract the inflammatory cells into the airways, activate them and maintain their presence. The cells that are present also release enzymes (like metalloproteases) and oxygen radicals which have a negative effect on tissue and perpetuate the disease. A vast array of pro-inflammatory cytokines and chemokines has been shown to be increased within the lungs of patients with COPD. Among them, an important role is played by tumor necrosis factor alpha (TNF-alpha), granulocyte-macrophage colony stimulating factor (GM-CSF) and interleukin 8 (IL-8), which are increased in the airways of patients with COPD.

Other examples of respiratory diseases where inflammation seems to play a role include: asthma, eosinophilic cough, bronchitis, cystic fibrosis, pulmonary hypertension, acute respiratory distress syndrome (ARDS), acute and chronic rejection of lung allograft, sarcoidosis, pulmonary fibrosis, rhinitis and sinusitis.

Asthma is defined by airway inflammation, reversible obstruction and airway hyperresponsiveness. In this disease the inflammatory cells that are involved are predominantly eosinophils, T lymphocytes and mast cells, although neutrophils and macrophages may also be important. A vast array of cytokines and chemokines have been shown to be increased in the airways and play a role in the pathophysiology of this disease by promoting inflammation, obstruction and hyperresponsiveness.

Eosinophilic cough is characterized by chronic cough and the presence of inflammatory cells, mostly eosinophils, within the airways of patients in the absence of airway obstruction or hyperresponsiveness. Several cytokines and chemokines are increased in this disease, although they are mostly eosinophil directed. Eosinophils are recruited and activated within the airways and potentially release enzymes and oxygen radicals that play a role in the perpetuation of inflammation and cough.

Acute bronchitis is an acute disease that occurs during an infection or irritating event for example by pollution, dust, gas or chemicals, of the lower airways. Chronic bronchitis is defined by the presence of cough and phlegm production on most days for at least 3 months of the year, for 2 years. One can also find during acute or chronic bronchitis within the airways inflammatory cells, mostly neutrophils, with a broad array of chemokines and cytokines. These mediators are thought to play a role in the inflammation, symptoms and mucus production that occur during these diseases.

Lung transplantation is performed in patients with end stage lung disease. Acute and more importantly chronic allograft rejection occur when the inflammatory cells of our body, lymphocytes, do not recognize the donor organ as “self”. Inflammatory cells are recruited by chemokines and cytokines and release a vast array of enzymes that lead to tissue destruction and in the case of chronic rejection a disease called bronchiolitis obliterans.

Sarcoidosis is a disease of unknown cause where chronic non-caseating granulomas occur within tissue. The lung is the organ most commonly affected. Lung bronchoalveolar lavage shows an increase in mostly lymphocytes, macrophages and sometimes neutrophils and eosinophils. These cells are also recruited and activated by cytokines and chemokines and are thought to be involved in the pathogenesis of the disease.

Pulmonary fibrosis is a disease of lung tissue characterized by progressive and chronic fibrosis (scarring) which will lead to chronic respiratory insufficiency. Different types and causes of pulmonary fibrosis exist but all are characterized by inflammatory cell influx and persistence, activation and proliferation of fibroblasts with collagen deposition in lung tissue. These events seem related to the release of cytokines and chemokines within lung tissue.

Acute rhinitis is an acute disease that occurs during an infection or irritating event, for example, by pollution, dust, gas or chemicals, of the nose or upper airways. Chronic rhinitis is defined by the presence of a constant chronic runny nose, nasal congestion, sneezing and pruritis. One can also find within the upper airways during acute or chronic rhinitis inflammatory cells with a broad array of chemokines and cytokines. These mediators are thought to play a role in the inflammation, symptoms and mucus production that occur during these diseases.

Acute sinusitis is an acute, usually infectious disease of the sinuses characterized by nasal congestion, runny, purulent phlegm, headache or sinus pain, with or without fever. Chronic sinusitis is defined by the persistence for more than 6 months of the symptoms of acute sinusitis. One can also find during acute or chronic sinusitis within the upper airways and sinuses inflammatory cells with a broad array of chemokines and cytokines. These mediators are thought to play a role in the inflammation, symptoms and phlegm production that occur during these diseases.

There is a growing body of evidence suggesting an intimate link between inflammation and neoplastic diseases. The tumor microenvironment is shaped by cells entering it, and their functions reflect the local conditions. Successive changes occurring at the tumor site during tumor progression resemble chronic inflammation. This chronic inflammatory reaction seems to be largely orchestrated by the tumor, and it seems to promote tumor survival. It has become evident that early and persistent inflammatory responses observed in or around developing neoplasms regulates many aspects of tumour development (matrix remodelling, angiogenesis, malignant potential) by providing diverse mediators implicated in maintaining tissue homeostasis, e.g., soluble growth and survival factors, matrix remodelling enzymes, reactive oxygen species and other bioactive molecules.

As described above, these inflammatory respiratory diseases or diseases in which inflammation plays a critical role are all characterized by the presence of mediators that recruit and activate different inflammatory cells which release enzymes or oxygen radicals causing symptoms, the persistence of inflammation and when chronic, destruction or disruption of normal tissue.

A therapeutic approach that would decrease pro-inflammatory cytokine and chemokine release by a vast array of cells while having a reduced effect on the release of anti-inflammatory mediators or enzymes may have an advantage over current therapies for inflammatory respiratory diseases or any other systemic inflammatory disease.

The cyclic nucleotides cAMP and cGMP are ubiquitous second messengers participating in signaling transduction pathways and mechanisms. Both cAMP and cGMP are formed from their respective triphosphates (ATP and GTP) by the catalytic activity of adenylyl (adenylate) or guanylyl (guanylate) cyclase. Inactivation of cAMP/cGMP is achieved by hydrolytic cleavage of the 3′-phosphodiester bond catalyzed by the cyclic-nucleotide-dependent phosphodiesterases (PDEs), resulting in the formation of the corresponding, inactive 5′-monophosphate. It has been shown that the inflammatory response and its progression is exquisitely sensitive to modulations in the steady-state levels of cyclic nucleotides, where target cells for their effects extend beyond immune cells to include accessory cells, such as airway smooth muscle, epithelial and endothelial cells, and neurons. The cyclic nucleotide PDEs are a large, growing multigene family, comprising at least 11 families of PDE enzymes. The profile of selective and nonselective PDE inhibitors in vitro and in vivo, therefore, suggests a potential therapeutic utility as antidepressants, antiproliferative, immunomodulatory, tocolytics, inotropes/chronotropes, and cytoprotective agents. Use of antisense oligonucleotides (“AONs”) directed against PDE are described in co-owned WO 05/030787 and WO 07/134,451.

For potential clinical uses, oligonucleotides should exhibit stability against degradation by serum and cellular nucleases, show low non-specific binding to serum and cell proteins, exhibit enhanced recognition of the target mRNA sequence, demonstrate cell-membrane permeability and elicited cellular nucleases when complexed with complementary mRNA. It is well documented that oligonucleotides containing natural sugars (D-ribose and D-2-deoxyribose) and phosphodiester (PO) linkages are rapidly degraded by serum and intracellular nucleases, which limit their utility as effective therapeutic agents. Chemical strategic modifications have been described for oligonucleotides in order to improve their stability and efficacy as therapeutic agents. The main chemical changes included, modification of the sugar moiety, the base moiety, and/or modification or replacement of the internucleotide phosphodiester linkage. To date the most widely studied analogues are the phosphorothioate (PS) oligodeoxynucleotides, in which one of the non-bridging oxygen atoms in the phosphodiester backbone is replaced with a sulfur.

For example, some PO linkages within immunostimulatory sequences such as CpG-containing oligonucleotides have been replaced with PS linkages to increase the stability of the oligonucleotide. However, generally PO linkages within the CpG motif are required and therefore a mixed PO/PS backbone is needed for maximal immunostimulatory activity of the CpG-containing oligonucleotides.

There are examples of antisense oligonucleotides with PO/PS linkages and incorporation of FANA modifications. However, such modifications can reduce the activity of the antisense oligonucleotide, as described in co-owned WO09/137,912.

Other modifications to oligonucleotides include the replacement of adenosine residues with 2-amino-2′-deoxyadenosine (DAP), as described in co-owned WO 03/004511.

It would be desirable to have improved oligonucleotides directed against PDEs for use in treating inflammation and respiratory diseases.

### SUMMARY OF THE INVENTION

In an aspect, there is provided an oligonucleotide directed against a target Phosphodiesterase (PDE), wherein the oligonucleotide is capable of hybridizing to at least a portion of a nucleic acid sequence encoding the PDE under stringent conditions, and wherein,
at least one nucleotide of the oligonucleotide is 2-amino-2′-deoxyadenosine (DAP); and
the internucleoside linkages of the oligonucleotide comprises at least three alternating segments, each segment consisting of either at least one phosphorothioate or at least one phosphodiester bond.

In a further aspect, there is provided a pharmaceutical composition comprising one oligonucleotide described herein and a pharmaceutically acceptable carrier.

In a further aspect, there is provided the pharmaceutical composition described herein for the treatment of inflammation.

In a further aspect, there is provided the pharmaceutical composition described herein for treatment of respiratory disease.

In a further aspect, there is provided a method of treating respiratory disease in a subject comprising administering the pharmaceutical composition described herein.

In a further aspect, there is provided the use of the pharmaceutical composition described herein in the preparation of a medicament for the treatment of respiratory disease.

In a further aspect, there is provided the use of the pharmaceutical composition described herein for the treatment of respiratory disease.

In a further aspect, there is provided an oligonucleotide comprising the base sequence of any one of SEQ ID NOs. 1-72.

In a further aspect, there is provided an oligonucleotide consisting of any one of SEQ ID NOs. 1-127.

### BRIEF DESCRIPTION OF THE DRAWINGS

FIG. 1 illustrates the efficacy of AON sequences at reducing the PDE7A mRNA expression. Flp-In T-Rex 293 cells were transfected for 24 h using 236 nM of AON sequences TOP1766 (SEQ. ID No. 30) to TOP1777 (SEQ. ID. No. 41) and efficacy compared to that of a known PDE7A AON TOP1731 (SEQ. ID No. 1). TOP1731s (SEQ. ID No. 128) was used as negative control. PDE7A mRNA expression (mean±SEM) was quantified using the Quantigene 2.0 assay, with normalization to the expression levels of the control gene β2M. The average percentage inhibition of PDE7A mRNA compared to control non-transfected cells (Ctrl NT, dotted line) was indicated for AON treatments when greater than 20%. N=6 for Ctrl NT and n=3 for transfected samples.

FIG. 2 illustrates the efficacy of AON sequences at reducing the PDE4D and PDE4B mRNA expression. Flp-In T-Rex 293 cells were transfected for 24 h using 236 nM of AON sequence against TOP1572 (SEQ. ID No. 42). TOP1572s (SEQ. ID No. 131) was used as negative control. (A) PDE4D and (B) PDE4B mRNA expression (mean±SEM) was quantified using the Quantigene 2.0 assay, with normalization to the expression levels of the control gene β2M. The average percentage inhibition of PDE4D and PDE4B mRNA compared to gene levels in control non-transfected cells (Ctrl NT, dotted line) was indicated for AON treatments when greater than 20%. N=6 for Ctrl NT and n=3 for transfected samples.

FIG. 3 illustrates the efficacy of human AON sequences at reducing mouse PDE mRNA expression. NIH3T3 cells were transfected for 24 h with 125 nM of the human PDE7A AON sequences TOP1731 (SEQ. ID No. 1), TOP1769 (SEQ. ID No. 33), TOP1777 (SEQ. ID No. 41) and the PDE4B/4D sequence TOP1572 (SEQ ID No. 42). The efficacy of the human AON was compared to that of known mouse specific PDE7A AON sequence TOP2707-F3 (SEQ. ID No. 134) and PDE4B/4D specific sequence TOP2804-F1 (SEQ. ID No.) or control sequences TOP2707Mi2-F3 (SEQ. ID No. 125) and TOP1572s (SEQ. ID No. 131). The Quantigene 2.0 assay was used to quantify murine (A) PDE7A, (B) PDE4D or (C) PDE4B mRNA expression (mean±SEM) normalized to β2M levels. The average percentage inhibition of PDE mRNA compared to gene levels in non-transfected control cells (Ctrl NT, dotted line) was indicated when greater than 20% (n=3 for all tested group, except n=6 for Ctrl NT).

FIG. 4 illustrates the efficacy of human AON sequences at reducing rat PDE mRNA expression. L2 cells were transfected for 24 h with 250 nM of PDE7A AON sequences TOP1731-P²M-7-DAP (SEQ. ID No. 86), TOP1769-P²M-7-DAP (SEQ. ID No. 95), TOP1777-P²M-7-DAP (SEQ. ID No. 101) or 500 nM of PDE4B/4D AON sequence TOP1572-P²M-7-DAP (SEQ ID No. 119). The efficacy of the human AON sequences was compared to that of control sequences TOP1731s-P²M-7-DAP (SEQ. ID No. 155) and TOP1572s-P²M-7-DAP (SEQ. ID No. 156). The Quantigene 2.0 assay was used to quantify rat (A) PDE7A, (B) PDE4D or (C) PDE4B mRNA expression (mean±SEM) normalized to PPIB levels. The average percentage inhibition of PDE mRNA compared to gene levels in non-transfected control cells (Ctrl NT, dotted line) was indicated when greater than 20% (n=3 for all tested group, except n=6 for Ctrl NT).

FIG. 5 illustrates the efficacy of human AON sequences at reducing monkey PDE mRNA expression. CYNOM-K1 cells were transfected for 24 h with 250 nM of PDE7A AON sequences TOP1731-P²M-7-DAP (SEQ. ID No. 86), TOP1769-P²M-7-DAP (SEQ. ID No. 95), TOP1777-P²M-7-DAP (SEQ. ID No. 101), or the PDE4B/4D sequence TOP1572 (SEQ ID No. 42) or control sequences TOP1731s-P²M-7-DAP (SEQ. ID No. 155) and TOP1572s (SEQ. ID No. 131). The Quantigene 2.0 assay was used to quantify monkey (A) PDE7A, (B) PDE4D or (C) PDE4B mRNA expression (mean±SEM) normalized to PPIB levels. The average percentage of PDE mRNA inhibition compared to gene levels in non-transfected control cells (Ctrl NT, dotted line) was indicated when greater than 20% (n=3 for all tested group, except n=6 for Ctrl NT).

FIG. 6 illustrates the efficacy comparison between selected unmodified AON (PS-DNA) and DAP-containing AON sequences at reducing target mRNA expression. Flp-In T-Rex 293 cells were transfected for 24 h using 118 nM of TOP1731 (SEQ. ID No. 1), TOP1731-DAP (SEQ. ID No. 74) or 263 nM of TOP1572 (SEQ. ID. No. 42) or TOP1572-DAP (SEQ. ID No. 107). (A) PDE7A, (B) PDE4D and C) PDE4B mRNA expression (mean±SEM) were quantified using the Quantigene 2.0 assay, with normalization to the expression levels of the control gene β2M. The average percentage of PDE mRNA inhibition compared to gene levels in control non-transfected cells (Ctrl NT, dotted line) was indicated for AON treatments. N=6 for Ctrl NT and n=3 for transfected samples.

FIG. 7 illustrates the efficacy comparison between selected P²M-DAP AON sequences at reducing target mRNA expression. FIp-In T-REx 293 cells were transfected for 24 h with 236 nM of PDE7A AON as PS-DNA or as various P²M or P²M-DAP versions of (A) TOP1731 (SEQ. ID No. 1), (B) TOP1769 (SEQ. ID No. 33) or (C) TOP1777 (SEQ. ID No. 41). The mRNA expression (mean±SEM) of PDE7A was quantified using the Quantigene 2.0 assay with normalization to the expression levels of β2M. The average percentage of PDE mRNA inhibition compared to gene level in non-transfected control (Ctrl NT, dotted line) is indicated when greater than 20%. Student t-test analyses, *p<0.05, **p<0.01, ***p<0.001. N=3 for all test groups except n=6 for Ctrl NT.

FIG. 8 illustrates the efficacy comparison between selected PS-DNA and P²M-DAP-containing AON sequences at reducing target mRNA expression. FIp-In T-REx 293 cells were transfected for 24 h with 15 to 1200 nM of (A) TOP1731 (SEQ. ID No. 1), (B) TOP1769 (SEQ. ID No. 33), (C) TOP1777 (SEQ. ID No. 41) or (D, E) TOP1572 (SEQ. ID No. 42) as PS-DNA or P²M-7-DAP, respectively. Sequences TOP1731s (SEQ. ID No. 128) and TOP1777s-P²M-7-DAP (SEQ. ID No. 132) were used as controls. The Quantigene 2.0 assay was used to quantify PDE mRNA expression (mean±SEM) relative to the expression of the control gene β2M (n=3 for all test groups except n=6 for Ctrl NT). Non-linear fit (one phase exponential decay) curves were generated using the GraphPad Prism software. Panel F illustrates the area under the dose-response curves (AUC) and compares the percentage reduction of the AUC between P²M-7-DAP-containing AON sequences and PS-DNA.

FIG. 9 illustrates the dose-response comparison between selected FANA-containing and P²M-DAP-containing AONs. FIp-In T-REx 293 cells were transfected for 24 h with 6 to 1260 nM of a combination of either FANA-containing or P²M-DAP-containing TOP1572 (SEQ. ID No. 42) and TOP1731 (SEQ. ID. No. 1). (A) PDE7A, (B) PDE4D and (C) PDE4B mRNA expression (mean±SEM) were measured using the Quantigene 2.0 assay, with normalization to the expression of the control gene β2M. Non-linear fit (one phase exponential decay) curves were generated using the GraphPad Prism software. The percentage of PDE inhibition at the maximal concentration compared to gene levels in non-transfected cells are indicated (n=6 for Ctrl NT and n=3 for transfected conditions).

FIG. 10 illustrates the comparison between selected LNA-containing and P²M-DAP-containing AONs. FIp-In T-REx 293 cells were transfected for 24 h with either 0, 238, 476 or 713 nM of a P²M-DAP-containing sequence (TOP1731-P²M-7-DAP, SEQ. ID No. 86) or LNA-containing sequence (TOP1731-LNA1, SEQ. ID No. 93). Sequence TOP1777s-P²M-7-DAP (SEQ. ID No. 132) was used as control. PDE7A mRNA expression (mean±SEM) was measured using the Quantigene 2.0 assay, with normalization to the expression of the control gene β2M. N=6 for Ctrl NT and n=3 for transfected conditions.

FIG. 11 illustrates the dose-response efficacy of selected PDE4B/4D and PDE7A P²M-DAP-containing AON combinations. Flp-In T-REx-293 cells were transfected using 6 to 1260 nM of indicated AON combinations or control sequence TOP1777s-P²M-7-DAP (SEQ. ID No. 132). (A) PDE7A, (B) PDE4D and (C) PDE4B mRNA expressions (mean±SEM) were measured using the Quantigene 2.0 assay and normalized to the expression of the control gene β2M. Non-linear fit (sigmoidal dose-response/variable slope) curves on transformed data (Log(X)) were generated using the GraphPad Prism software.

FIG. 12 illustrates the tissue half-lives of selected PDE4B/4D and PDE7A AON in mouse lung. The amount of remaining TOP1572-P²M-7-DAP (SEQ. ID No. 119), TOP1731-P²M-7-DAP (SEQ. ID No. 86), TOP1769-P²M-7-DAP (SEQ. ID No. 95) and TOP1777-P²M-7-DAP (SEQ. ID No. 101) was determined in lung tissue homogenates at different time points following administration via intra-tracheal aerosols. Each data point represents the mean±SD of 5 mice per time point, and is expressed as the percentage of amount of AON recovered relative to time 0 h set at 100%. Non-linear regression curves were generated using Graph Pad Prism and the half-life of each AON was calculated using one-phase exponential decay parameters.

FIG. 13 illustrates the duration of action of selected PDE4B/4D and PDE7A P²M-DAP-containing AON combinations. Flp-In T-REx-293 cells were transfected using 378 nM of indicated AON combinations (PDE4B/4D; TOP1572-P²M-7-DAP (SEQ. ID No. 119), PDE7A; TOP1731-P²M-7-DAP (SEQ. ID No. 86), TOP1769-P²M-7-DAP (SEQ. ID No. 95), TOP1777-P²M-7-DAP (SEQ. ID No. 101)) or control sequence TOP1777s-P²M-7-DAP (SEQ. ID No. 132). (A) PDE7A, (B) PDE4D and (C) PDE4B mRNA expressions (mean±SEM) were measured at indicated time-points using the Quantigene 2.0 assay and normalized to the expression of the control gene β2M. Data are expressed on a log-time scale as mean±SEM with the control non-transfected (Ctrl NT) values set at 1.0. Paired t-test was used to calculate statistical difference versus TOP1572-P²M-7-DAP+TOP1731-P²M-7-DAP (*p<0.05, **p<0.01, n=3).

FIG. 14 shows the biological effect on AON on primary lung epithelial cells. Normal human bronchoepithelial (NHBE) cells were transfected for 20 h with a combination of PDE4B/4D- and PDE7A-specific P²M-DAP modified AON (250 nM total AON) (TOP1572-P²M-7-DAP (SEQ. ID No. 119) referred as TOP1572 on the “x”-axis of the figure, TOP1769-P²M-7-DAP (SEQ. ID No. 95) referred as TOP1769 on the “x”-axis of the figure) or control sequence (TOP1777s-P²M-7-DAP (SEQ. ID No. 132) referred as TOP1777s on the “x”-axis of the figure) and were stimulated with a cytomix (10 ng/mL each of TNF-α, IL-1β and IFN-γ) for 4 h to induce gene expression and protein secretion of chemokine (MCP-1), metalloproteases (MMP-2, MMP-12) and cytokine (GM-CSF, IL1-β). Rolipram (36 μM), a small molecule PDE4 inhibitor, was used as control. (A-G) Real-time PCR was used to quantify mRNA expression (mean±SEM) which was normalized to the expression of a control gene (β2M). N=6 for Ctrl NT and n=3 for transfected conditions. The percentage of inhibition compared to stimulated non-transfected cells (Ctrl NT, dotted line) are indicated. Ctrl NS represents non-transfected and non-stimulated cells. Student t-test was used to calculate statistical difference versus TOP1777s-P²M-7-DAP (*p<0.05, **p<0.01, ***p<0.001, n=3).

FIG. 15 shows the biological effect on AON on lung epithelial cells. A549 cells were transfected for 20 h with a combination of PDE4B/4D- and PDE7A-specific P²M-DAP modified AON (236 nM total AON) (PDE4B/4D; TOP1572 (SEQ. ID No. 42), TOP1572-P²M-7-DAP (SEQ. ID No. 119), PDE7A; TOP1731 (SEQ. ID No. 1), TOP1731-P²M-7-DAP (SEQ. ID No. 86), TOP1769-P²M-7-DAP (SEQ. ID No. 95) or TOP1777-P²M-7-DAP (SEQ. ID No. 101)) and were stimulated with IL-1β for 6 h to induce protein secretion of chemokines (IL-8 and MCP-1). Rolipram (36 μM), a small molecule PDE4 inhibitor, was used as control. Cell supernatants were harvested and levels (mean±SEM) of (A) IL-8 and (B) MCP-1 determined using ELISA. N=6 for Ctrl NT and n=3 for transfected conditions. The percentage of inhibition compared to stimulated non-transfected cells (Ctrl NT, dotted line) are indicated. Ctrl NS represents non-transfected and non-stimulated cells. Statistical significance is shown as ** (p<0.01). One-way ANOVA followed by Dunnett's post-test vs. stimulated cells (Ctrl NT).

FIG. 16 illustrates a comparison of the effects of P²M-DAP modified AONs to non-P²M-DAP modified AONs (PS-DNA) on the immune response in vitro. (A) Mouse macrophage cells (RAW 264.7) or (B) human PBMC were exposed to different concentrations of AONs (TOP1731 (SEQ. ID No. 1) or TOP1731-P²M-7-DAP (SEQ. ID No. 86)). Mouse macrophage cells (RAW 264.7) were also exposed to AON sequences TOP062 (SEQ. ID No.) or TOP062-P²M-11-DAP (SEQ. ID No.), TOP062-P²M-12-DAP (SEQ. ID No.), TOP057-P²M-4-DAP (SEQ. ID No.) and TOP057-P²M-5-DAP (SEQ. ID No.) against β-chain (C) or AONs TOP030 (SEQ. ID No.) or TOP030-P²M-15-DAP (SEQ. ID No.), TOP031-P²M-1-DAP (SEQ. ID No.) and TOP031-P²M-5-DAP (SEQ. ID No.) against CCR3 (D). A known immunostimuatory sequence CpG (SEQ. ID No. 133) was used as positive control and levels of TNF-α and IFN-α in culture supernatants measured 24 h later (n=3-5).

FIG. 17 illustrates a comparison of the effects of P²M-DAP modified AONs to non-P²M modified AONs (PS-DAP or PS-DNA) on the inflammatory response in the lung of rodents (mice or rat) following chronic dosing. (A) Rats were treated with selected AONs (PS-DAP; TOP004 (SEQ. ID No. 157), TOP005 (SEQ. ID No. 158), PS-DNA; TOP006 (SEQ. ID No. 159), TOP007 (SEQ. ID No. 160)) at the indicated dose for 14 consecutive days and the inflammatory response assessed by lung tissue histopathology. Mice were treated with selected AONs (TOP1731 (SEQ. ID No. 1) or TOP1731-P²M-7-DAP (SEQ. ID No. 86)) at indicated doses for 10 days and inflammatory response assessed in (B) bronchoalveolar lavage (BAL) and by (C) lung tissue histopathology.

FIG. 18 shows the effect of specific inhibition by P²M-DAP-modified PDE4B/4D and PDE7A AON on LPS-induced acute lung inflammation in mice. Inhibition of the cellular influx in the lung of LPS-exposed mice pre-treated with selected mouse (A) or human (B) AON combinations (PDE4B/4D; TOP2804-P²M-7-DAP (SEQ. ID No. 138), TOP1572-P²M-7-DAP (SEQ. ID No. 119), PDE7A; TOP2707-P²M-7-DAP (SEQ. ID No. 136), TOP1777-P²M-7-DAP (SEQ. ID No. 101)) or control sequence TOP1777s-P²M-7-DAP (SEQ. ID No. 132) or (C) roflumilast as positive control. Differential cell counts of BAL were performed 3 h after LPS exposure and percentage inhibition was determined by comparing with LPS-challenged vehicle treated animals. Values shown represent mean±SEM (n=5-10), Student t-test was used to calculate statistical difference versus LPS-challenged vehicle treated animals (*p<0.05, **p<0.01).

FIG. 19 shows the efficacy of treatment with combined PDE4B/4D and 7A P²M-DAP-modified AONs against cigarette smoke-induced lung inflammation. Inhibition of the cellular influx in the lung of smoke-exposed mice pre-treated with selected mouse (A and D) or human (B and E) AON combinations (PDE4B/4D; TOP2804-P²M-7-DAP (SEQ. ID No. 138), TOP1572-P²M-7-DAP (SEQ. ID No. 119), PDE7A; TOP2707-P²M-7-DAP (SEQ. ID No. 136), TOP1777-P²M-7-DAP (SEQ. ID No. 101)) or control sequence TOP1777s-P²M-7-DAP (SEQ. ID No. 132) or (C and F) roflumilast as positive control. Differential cell counts of BAL were performed 16-20 h after smoke exposure and percentage inhibition was determined by comparing with smoke-exposed vehicle treated animals. Values shown represent mean±SEM.

FIG. 20 illustrates the efficacy comparison between selected DAP-containing and P²M-DAP-containing AON sequences at reducing target mRNA and protein expression. (A and B) TF-1 cells were transfected for 24 h with 1.34 μM of a combination of either TOP004 (SEQ. ID No. 157) and TOP005 (SEQ. ID No. 158), or TOP062-P²M-12-DAP (SEQ. ID No. 185) and TOP030-P²M-15-DAP (SEQ. ID No. 170), or TOP062-P²M-12-DAP (SEQ. ID No. 185) and TOP031-P²M-1-DAP (SEQ. ID No. 172). The Quantigene 2.0 assay was used to quantify (A) CCR3 and (B) β-chain mRNA expression and represented as the % inhibition compared to the expression of the control untransfected cells. (C and D) TF-1 cells were transfected for 24 h with 334 or 668 nM total of a combination of either TOP004 (SEQ. ID No. 157) and TOP005 (SEQ. ID No. 158), or TOP062-P²M-12-DAP (SEQ. ID No. 185) and TOP030-P²M-15-DAP (SEQ. ID No. 170), or TOP062-P²M-12-DAP (SEQ. ID No. 185) and TOP031-P²M-1-DAP (SEQ. ID No. 172). Protein expression levels were measured by flow cytometry 24 h after transfection. Results are expressed as the mean fluorescence intensity (MFI)±SEM (n=3). Percentage inhibition compared to control non-transfected (Ctrl NT) cells indicated above each bar.

### BRIEF DESCRIPTION OF THE TABLES

Table 1 identifies AON sequences with specificity for the human phosphodiesterase (PDE) isoform 7A in accordance with the present invention.

Table 2 identifies AON sequences with specificity for the human phosphodiesterase (PDE) isoforms 4B and 4D in accordance with the present invention.

Table 3 illustrates the homology between human AON sequences and the sequences from different animal species (mouse, rat and monkey).

Table 4 identifies the AON sequences with specificity for the human phosphodiesterase (PDE) isoforms 4B, 4D and 7A containing modified chemistry in accordance with the present invention.

Table 5 identifies the oligonucleotide sequences used as control in accordance with the present invention.

Table 6 identifies AON sequences with specificity for the mouse phosphodiesterase (PDE) isoforms in accordance with the present invention.

Table 7 describes the real-time PCR primers used to quantify target genes and inflammatory markers in in vitro and in vivo models.

Table 8 describes the influx of inflammatory cells in bronchoalveolar lavages and the histopathological changes following administration of the AON sequences to the lungs of mice.

Table 9 identifies AON sequences used in in vivo toxicology studies in accordance with the present invention.

Table 10 identifies the AON sequences with specificity for the human (3-chain and CCR3 containing modified chemistry in accordance with the present invention.

### DETAILED DESCRIPTION

There is described herein oligonucleotides directed against phosphodiesterase (PDE) isoforms in order to downregulate the expression thereof. The oligonucleotides have a mixed phosphodiester/phosphorothioate backbone (P²M) and at least one 2-amino-2′-deoxyadenosine (DAP) nucleotide. It has been surprisingly found that the P²M and DAP modifications exhibit synergy in improving anti-PDE oligonucleotides by increasing their potency, efficacy or stability and/or decreasing their toxicity. For example, the oligonucleotides described herein preferably exhibit unexpectedly higher efficacy/toxicity ratios.

Accordingly, in an aspect, there is provided an oligonucleotide directed against a target Phosphodiesterase (PDE), wherein the oligonucleotide is capable of hybridizing to at least a portion of a nucleic acid sequence encoding the PDE under stringent conditions, and wherein,
at least one nucleotide of the oligonucleotide is 2-amino-2′-deoxyadenosine (DAP); and
the internucleoside linkages of the oligonucleotide comprises at least three alternating segments, each segment consisting of either at least one phosphorothioate or at least one phosphodiester bond.

Preferably, the target PDE is selected from the group consisting of PDE3A, PDE3B, PDE4A, PDE4B, PDE4C, PDE4D, PDE7A1, PDE7A2, PDE7A3 and PDE7B, preferably PDE7A, PDE4B and PDE4D.

In some embodiments, ratio of phosphorothioate to phosphodiester bonds is between 30:70 and 70:30, preferably 40:60 and 60:40, 45:55 and 55:45 and 50:50.

In some embodiments, the oligonucleotide is at least 80% complementary to the target PDE, preferably 100%.

The oligonucleotide preferably exhibits a efficacy/toxicity ratio greater than 0.25.

In some embodiments, the oligonucleotide is between 15-25 nucleotides in length, preferably between 18-22 nucleotides in length.

In one embodiment, the oligonucleotide has a base sequence selected from the group consisting of SEQ ID NOs. 1-72, wherein at least one adenosine is replaced with DAP, and is preferably SEQ ID NOs. 1, 33, 36, 41 and 42.

In a preferably embodiment, the oligonucleotide comprises, preferably consists of, any one of SEQ ID NOs. 81, 86, 90, 92, 95, 97, 99, 101, 103, 105, 114, 119, 121, 123, 125 and 127.

In a further aspect, there is provided a pharmaceutical composition comprising one oligonucleotide described herein and a pharmaceutically acceptable carrier. In one embodiment, the pharmaceutical compositions comprises at least two of the oligonucleotides described herein, preferably directed against PDE7A and PDE4B, or PDE7A and PDE4D respectively. In a preferably embodiment, the oligonucleotide directed against PDE4B or PDE4D also downregulates the other.

In a further aspect, there is provided the pharmaceutical composition described herein for the treatment of inflammation.

In a further aspect, there is provided the pharmaceutical composition described herein for treatment of respiratory disease, preferably at least one of chronic obstructive pulmonary disease, asthma, eosinophilic cough, bronchitis, acute and chronic rejection of lung allograft, sarcoidosis, pulmonary fibrosis, rhinitis, sinusitis, viral infection or a neoplastic disease.

In a further aspect, there is provided a method of treating respiratory disease in a subject comprising administering the pharmaceutical composition described herein.

In a further aspect, there is provided the use of the pharmaceutical composition described herein in the preparation of a medicament for the treatment of respiratory disease.

In a further aspect, there is provided the use of the pharmaceutical composition described herein for the treatment of respiratory disease.

In a further aspect, there is provided an oligonucleotide comprising the base sequence of any one of SEQ ID NOs. 1-72, preferably wherein at least one adenosine is replaced with DAP.

In a further aspect, there is provided an oligonucleotide consisting of any one of SEQ ID NOs. 1-127.

The terms “nucleic acid” and “nucleic acid molecule” as used interchangeably herein, refer to a molecule comprised of nucleotides, i.e., ribonucleotides, deoxyribonucleotides, or both. The term includes monomers and polymers of ribonucleotides and deoxyribonucleotides, with the ribonucleotide and/or deoxyribonucleotides being connected together, in the case of the polymers, via 5′ to 3′ linkages. However, linkages may include any of the linkages known in the nucleic acid synthesis art including, for example, nucleic acids comprising 5′ to 2′ linkages. The nucleotides used in the nucleic acid molecule may be naturally occurring or may be synthetically produced analogues that are capable of forming base-pair relationships with naturally occurring base pairs.

“Bases” includes any one of the natively found purine and pyrimidine bases, adenine (A), thymine (T), cytosine (C), guanine (G) and uracil (U), but also any modified or analogous forms thereof. Examples of non-naturally occurring bases that are capable of forming base-pairing relationships include, but are not limited to, aza and deaza pyrimidine analogues, aza and deaza purine analogues, and other heterocyclic base analogues, wherein one or more of the ring atoms and/or functional groups of the purine and pyrimidine rings have been substituted by heteroatoms, e.g., carbon, fluorine, nitrogen, oxygen, sulfur, and the like. Preferably, such bases include, but are not limited to, inosine, 5-methylcytosine, 2-thiothymine, 4-thiothymine, 7-deazaadenine, 9-deazaadenine, 3-deazaadenine, 7-deazaguanine, 9-deazaguanine, 6-thioguanine, isoguanine, 2,6-diaminopurine, hypoxanthine, and 6-thiohypoxanthine. Bases may also include, but are not limited to, 5-fluorocytosine, 5-bromocytosine, 5-iodocytosine, isocytosine, N4-methylcytosine, 5-iodouracil, 5-fluorouracil, 4-thiouracil, 2-thiouracil, (E)-5-(2-bromovinyl)uracil, N6-methyladenine, 2-chloroadenine, 2-fluoroadenine, 2-chloroadenine, N6-cyclopropyl-2,6-diaminopurine, nicotinamide, 2-aminopurine, 1,2,4-triazole-3-carboxamide.

The term “nucleic acid backbone” or “internucleoside linkage” as used herein refers to the structure of the chemical moiety linking nucleotides in a molecule. This may include structures formed from any and all means of chemically linking nucleotides.

The term “oligonucleotide” as used herein refers to a nucleic acid molecule from about 2 to about 100 nucleotides, and in increasing preferability, 2 to 80 nucleotides, 4 to 35 nucleotides, 10 to 30 nucleotides, 15 to 25 nucleotides, and 18 to 22 nucleotides.

Moreover, the skilled artisan recognizes that substantially similar nucleic acid sequences encompassed by this invention are also defined by their ability to hybridize, under stringent conditions (for example, 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions involves a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of highly stringent conditions involves the use of higher temperatures in which the washes are identical to those above except the temperature of the final two 30 min. washes in 0.2×SSC, 0.5% SDS was increased to 60° C. Another preferred set of very highly stringent conditions involves the use of two final washes in 0.1×SSC, 0.1% SDS at 65° C.

The terms “treatment”, “treating”, “therapy” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or amelioration of an adverse effect attributable to the disease. “Treatment” as used herein covers any treatment of a disease in a subject as previously defined, particularly a human, and includes:
(a) preventing a disease from occurring in a subject which may be predisposed to the disease but has not yet been diagnosed as having it;
(b) inhibiting a disease, i.e., arresting its development; or
(c) relieving a disease, i.e., causing regression of the disease.

The term “pharmaceutically acceptable” as it is used herein with respect to carriers, excipients, surfactants and compositions refers to substances which are acceptable for use in the treatment of a subject patient that are not toxic or otherwise unacceptable for administration by any of the routes herein described.

The formulations of the present invention are preferably administered directly to the site of action and, thus, preferably are topical, including but not limited to, oral, intrabuccal, intrapulmonary, rectal, intrauterine, intratumor, nasal, intrathecal, inhalable, transdermal, intradermal, intracavitary, iontophoretic, ocular, vaginal, intraarticular, otical, transmucosal, rectal, slow release or enteric coating formulations. Without limiting any of the foregoing, formulations of the present invention may also be intracranial, intramuscular, subcutaneous, intravascular, intraglandular, intraorgan, intralymphatic, intraperitoneal, intravenous, and implantable. The carriers used in the formulations may be, for example, solid and/or liquid carriers.

Reference may be made to “Remington's Pharmaceutical Sciences”, 17th Ed., Mack Publishing Company, Easton, Pa., 1985, for other carriers that would be suitable for combination with the present oligonucleotide compounds to render compositions/formulations suitable for administration to treat respiratory disease.

Optionally, the presently described oligonucleotides may be formulated with a variety of physiological carrier molecules. The presently described oligonucleotides may also be complexed with molecules that enhance their ability to enter the target cells. Examples of such molecules include, but are not limited to, carbohydrates, polyamines, amino acids, peptides, lipids, and molecules vital to cell growth. For example, the oligonucleotides may be combined with a lipid, the resulting oligonucleotide/lipid emulsion, or liposomal suspension may, inter alia, effectively increase the in vivo half-life of the oligonucleotide.

The pharmaceutical compositions provided herein may comprise oligonucleotide compounds described above and one or more pharmaceutically acceptable surfactants. Suitable surfactants or surfactant components for enhancing the uptake of the oligonucleotides of the invention have been previously described in U.S. Application Publication No. 2003/0087845, the contents of which are incorporated herein with respect to surfactants. The application states that suitable surfactants “ . . . include synthetic and natural as well as full and truncated forms of surfactant protein A, surfactant protein B, surfactant protein C, surfactant protein D and surfactant protein E, di-saturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine; phosphatidic acid, ubiquinones, lysophosphatidylethanolamine, lysophosphatidylcholine, palmitoyl-lysophosphatidylcholine, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate; as well as natural and artificial lamelar bodies which are the natural carrier vehicles for the components of surfactant, omega-3 fatty acids, polyenic acid, polyenoic acid, lecithin, palmitinic acid, non-ionic block copolymers of ethylene or propylene oxides, polyoxypropylene, monomeric and polymeric, polyoxyethylene, monomeric and polymeric, poly (vinyl amine) with dextran and/or alkanoyl side chains, Brij 35™, Triton X-100™ and synthetic surfactants ALEC™, Exosurf™, Survan™ and Atovaquone™, among others. These surfactants may be used either as single or part of a multiple component surfactant in a formulation, or as covalently bound additions to the 5′ and/or 3′ ends of the AONs.

The oligonucleotide component of the present compositions may be contained in a pharmaceutical formulation within a lipid particle or vesicle, such as a liposome or microcrystal. As described in U.S. Pat. No. 6,025,339, the lipid particles may be of any suitable structure, such as unilamellar or plurilamellar, so long as the oligonucleotide is contained therein. Positively charged lipids such as N-[1-(2,3-dioleoyloxi) propyl]-N,N,N-trimethyl-ammoniumethylsulfate, or “DOTAP,” are particularly preferred for such particles and vesicles. The preparation of such lipid particles is well known. See, e.g., U.S. Pat. No. 4,880,635 to Janoff et al.; U.S. Pat. No. 4,906,477 to Kurono et al.; U.S. Pat. No. 4,911,928 to Wallach; U.S. Pat. No. 4,917,951 to Wallach; U.S. Pat. No. 4,920,016 to Allen et al.; U.S. Pat. No. 4,921,757 to Wheatley et al.; etc.

The composition of the invention may be administered by any means that transports the oligonucleotide compound to the desired site, such as for example, the lung. The oligonucleotide compounds disclosed herein may be administered to the lungs of a patient by any suitable means, but are preferably administered by inhalation of an aerosol comprised of respirable particles that comprise the oligonucleotide compound.

The oligonucleotides may be formulated to be administered in a dry powder inhaler, metered dose inhaler, nebulizer, soft mist inhaler and by any other suitable device having the capacity to deliver oligonucleotides to the lungs via inhalation route.

The composition of the present invention may be administered into the respiratory system as a formulation including particles of respirable size, e.g. particles of a size sufficiently small to pass through the nose, mouth and larynx upon inhalation and through the bronchi and alveoli of the lungs. In general, respirable particles range from about 0.5 to 10 microns in size. Particles of non-respirable size that are included in the aerosol tend to deposit in the throat and be swallowed, and the quantity of non-respirable particles in the aerosol is preferably thus minimized. For nasal administration, a particle size in the range of 10-500 μM is preferred to ensure retention in the nasal cavity.

A solid particulate composition comprising the oligonucleotide compound may optionally contain a dispersant that serves to facilitate the formation of an aerosol as well as other therapeutic compounds. A suitable dispersant is lactose, which may be blended with the antisense compound in any suitable ratio, e.g., a 1 to 1 ratio by weight.

Liquid pharmaceutical compositions of active compound (the oligonucleotide compound(s)) for producing an aerosol may be prepared by combining the oligonucleotide compound with a suitable vehicle, such as sterile pyrogen free water or phosphate buffered saline.

The aerosols of liquid particles comprising the oligonucleotide compound may be produced by any suitable means, such as with a nebulizer. Nebulizers are commercially available devices that transform solutions or suspensions of the active ingredient into a therapeutic aerosol mist either by means of acceleration of a compressed gas, typically air or oxygen, through a narrow venturi orifice or by means of ultrasonic agitation. Suitable formulations for use in nebulizers comprise the active oligonucleotide ingredient in a liquid carrier in an amount of up to 40% w/w preferably less than 20% w/w of the formulation. The carrier is typically water or a dilute aqueous alcoholic solution, preferably made isotonic with body fluids by the addition of, for example, sodium chloride. Optional additives include preservatives if the formulation is not prepared sterile, for example, methyl hydroxybenzoate, anti-oxidants, anti-bacterials, flavorings, volatile oils, buffering agents and emulsifiers and other formulation surfactants.

The aerosols of solid particles comprising the active oligonucleotide compound(s) and a pharmaceutically acceptable surfactant may likewise be produced with any solid particulate medicament aerosol generator. Aerosol generators for administering solid particulate medicaments to a subject produce particles that are respirable, as explained above, and generate a volume of aerosol containing a predetermined metered dose of a medicament at a rate suitable for human administration. The active oligonucleotide ingredient typically comprises from 0.1 to 100 w/w of the formulation. A second type of illustrative aerosol generator comprises a metered dose inhaler. Metered dose inhalers are pressurized aerosol dispensers, typically containing a suspension or solution formulation of the active ingredient in a liquified propellant. During use these devices discharge the formulation through a valve adapted to deliver a metered volume, typically from 10 to 150 μL, to produce a fine particle spray containing the active ingredient. Suitable propellants include certain chlorofluorocarbon compounds, for example, dichlorodifluoromethane, trichlorofluoromethane, dichlorotetrafluoroethane or hydrofluoroalkanes and mixtures thereof. The formulation may additionally contain one or more co-solvents, for example, ethanol, emulsifiers and other formulation surfactants, such as oleic acid or sorbitan trioleate, anti-oxidants and suitable flavoring agents.

The aerosol, whether formed from solid or liquid particles, may be produced by the aerosol generator at a rate of from about 1 to 150 litres per minute.

The present invention will be more readily understood by referring to the examples that are given to illustrate the following invention rather than to limit its scope.

### EXAMPLES

### Methods

### Cell Culture

A549 cells (human lung carcinoma; ATCC; #CCL-185) were grown in Ham's F12 media containing 10% FBS, 100 U/mL Penicillin and 100 μg/mL Streptomycin. Flp-In T-Rex 293 cells (human transformed embryonic kidney; Invitrogen; #R780-07) were cultivated in DMEM containing 10% FBS, penicillin 100 U/mL and streptomycin 100 μg/mL. NHBE primary cells (normal human bronchial epithelial; Cederlane; #CC-2540) were cultivated in BEBM media (500 mL) supplemented with 2 ml BPE, 0.5 mL Hydrocortisone, 0.5 mL hEGF, 0.5 mL Epinephrine, 0.5 mL Transferrin, 0.5 mL Insulin, 0.5 mL Retinoic Acid, 0.5 mL Triiodothyronine (Cederlane, cat#CC-3170). CYNOM-K1 cells (cynomolgus monkey skin embryonic; ECACC; #90071809) were cultivated in EMEM (EBSS) media containing 10% FBS, 2 mM L-glutamine, 1% non-essential amino acids, 100 U/mL Penicillin and 100 μg/mL Streptomycin. L2 cells (rat lung epithelial; ATCC; #CRL-149) were cultivated in F12K media containing 10% FBS, penicillin 100 U/mL and streptomycin 100 μg/mL. NIH3T3 cells (mouse fibroblast; ATCC; #CRL-1658) were cultivated in DMEM media containing 10% BCS, penicillin 100 U/mL and streptomycin 100 μg/mL. RAW264.7 cells (Leukaemic monocyte macrophage; ATCC; #TIB-71) were cultivated in DMEM media containing 10% FBS, 1 mM sodium pyruvate, penicillin 100 U/mL and streptomycin 100 μg/mL. Human peripheral blood mononuclear cells (PBMC) were obtained from healthy volunteers. PBMC were isolated by Ficoll-Hypaque density gradient centrifugation of EDTA K3 blood from normal donors. PBMC were plated at 0.2×10⁶ cells/200 μL in 48-well plates (for suspension cells) in AIM-V culture media. TF-1 cells (erythroleukemia) were maintained in RPMI-1640 supplemented with 2 mM L-glutamine, 10 mM HEPES, 4.5 g/L glucose, 1.5 g/L sodium bicarbonate, 1 mM sodium pyruvate, 10% non-inactivated FBS and 2 ng/mL rhGM-CSF.

### Preparation of Antisense Oligonucleotides (AON)

Phosphorothioate-DNA (PS-DNA) AON (Sigma Genosys), Phosphorothioate-FANA (PS-FANA) AON (Topigen, Montreal or UcDNA, Calgary), DAP, P²M and P²M-DAP AON (Biospring, Germany), 2′-OMe AON (Biospring, Germany) or Lock nucleic acid (LNA) AON (Exiqon, USA) were resuspended in sterile water and diluted in Opti-MEM for in vitro transfection or diluted in PBS for in vivo studies.

### AON Transfection of Cells

A549 cells were seeded at 0.25×10⁶ cells/mL (200 μL per well in 48-well plates) in HAM-F12 medium supplemented with 10% serum without antibiotics then incubated overnight at 37° C. prior to transfection with AON/Lipofectamine complexes (ratio of 1 μg AON:1 μL Lipofectamine 2000). Flp-In T-Rex 293 cells were seeded at 0.9×10⁶ cells/mL (200 μL per well in 48-well plates) in DMEM medium supplemented with 10% serum without antibiotics then incubated overnight at 37° C. prior to transfection as described above. NHBE cells were seeded at 0.5×10⁶ cells/mL (200 μL per well in 48-well plates) in BEBM complete medium without antibiotics for 24 h prior to transfection as described. L2 cells were seeded at 1.75×10⁶ cells/mL (400 μL per well in 24-well plates) in F12K medium supplemented with 10% serum without antibiotics then incubated overnight at 37° C. and transfected as described above. CYNOM-K1 cells were seeded at 0.25×10⁶ cells/mL (200 μL per well in 48-well plates) in EMEM medium supplemented with 10% serum without antibiotics then incubated overnight at 37° C. prior to transfection with AON/Lipofectamine complexes (ratio of 1 μg AON:2 μL Lipofectamine 2000). NIH 3T3 cells were seeded at 0.25×10⁶ cells/mL (200 μL per well in 48-well plates) in DMEM medium supplemented with 10% serum without antibiotics then incubated overnight at 37° C. prior to transfection with AON/Lipofectamine complexes (ratio of 1 μg AON:3 μL Lipofectamine 2000). The day before the transfections, TF-1 cells were diluted as to reach a density between 0.6 and 0.8×10⁶ cells/mL the following day. The day of transfection, cells were plated in 12 well-plates at a density of 0.5×10⁶ cells/well in 400 μL growth media without antibiotics and then transfected with AONs mixed with the Lipofectamine 2000 transfection reagent according to a ratio of 1 μg nucleic acid:1 μl lipid. The complexes were incubated for 20 min. and added to the cells for 24 h treatment at 37° C., 5% CO₂.

### RNA Extraction

RNA was extracted from cell pellets according to the RNAeasy mini kit protocol using the QiaVac 24 manifold from Qiagen and treated with DNase according to Qiagen's procedure. RNA was quantified using the RiboGreen reagent according to the manufacturer's protocol (Invitrogen).

### Reverse Transcription (RT)

Preparation of first-strand cDNA was performed using the Superscript First-Strand Synthesis System for RT-PCR kit (Invitrogen), in a total reaction volume of 20 μL. 1 μg of RNA was first denatured at 65° C. for 5 min., with 0.5 mM of each dNTPs, 0.5 μg of oligo (dT)₁₈, 2 pmol of PDE4B gene specific reverse primer (5′-tctttgtctccctgctgga-3′) and chilled on ice for at least 1 min. The mixture was incubated at 42° C. for 2 min. and a second pre-mix containing 1× First-Strand Buffer, 10 mM DTT, and 40 units of SuperScript II RT was added. Reactions were incubated at 42° C. for 10 min., at 50° C. for 1 h and inactivated by heating at 70° C. for 15 min.

### Real-time PCR

PCR reaction mixtures were performed with 3 □L of cDNA reaction in a total volume of 20 μL in presence of 0.4 mM of each PCR primer and 4 μL of LC FastStart DNA Master SYBR Green 1 PLUS. Step 1 (Denaturation): 95° C., 10 min (slope 20° C./sec); Step 2 (Cycles×40): 95° C., (slope 20° C./sec); 57° C. or 59° C., 5 sec. (slope 20° C./sec) 72° C., 10 sec. (slope 20° C./sec); Step 3 (Melting curve):95° C., (slope 20° C./sec); 70° C., 30 sec. (slope 20° C./sec); 95° C., 0 sec. (slope 0.1° C./sec); Step 4 (Cool): 40° C., 30 sec (slope 20° C./sec)

PCR primer sequences used for each gene are described in Table 6. Quantification of PCR products was performed using the RelQuant program (Roche).

### Quantigene Assay

Cells were harvested and lysed for 30 min at 55° C. in 1× lysis mixture from the Quantigene® 2.0 kit (Affymetrix). Cell lysates were hybridized overnight at 55° C. in Quantigene® 2.0 capture plates in the presence of specific probe sets and mRNA signal was amplified (at 55° C.) and detected (at 50° C.). mRNA expression was linearly quantified by luminescence signal using the Luminoskan.

### Protein Quantification

For β-chain or CCR3 protein measurements, TF-1 were harvested 24 h post-transfection, washed twice with PBS, and incubated with eotaxin-biotin or IL-3-biotin for 1 h at 4° C. Avidin-FITC was then added and cells incubated for 30 min at 4° C. Cells were washed twice with PBS and fixed in 4% paraformaldehyde before flow cytometry analysis.

### In Vitro Functional Assays

NHBE cells were stimulated for 4 h with 10 ng/mL cytomix (TNF-α+IL-1β+IFN-γ) at the end of the transfection period. A549 cells were stimulated for 6 h with 10 ng/mL IL-1β at the end of the transfection period. The mRNA expression of inflammatory markers was analyzed by real-time PCR. The protein secretion of was measured in cells supernatant by multiplex ELISA (SearchLight service, Aushon), with normalization to the cell viability as measured by alamarBlue assay.

### In Vitro Immunostimulation Assays

RAW264.7 cells were seeded at 0.13×10⁶ cells/mL in complete media without antibiotics in 24-well plates and incubated in the presence of AON sequences or control CpG immunostimulatory sequences for 24 h. PBMC were seeded at 1×10⁶ cells/mL with AIM-V media without antibiotics in 48-well plates and treated as described above. Following treatment, culture supernatants were collected and protein content assayed by ELISA following the manufacturer's directions.

### Half-Life Determination in Mouse Lung

CD-1 male mice (25-30 g, Charles River) were acclimated to the animal facility (Mispro, Montreal, Canada) for approximately one week prior to the beginning of treatment. Mice were anesthesized with isoflurane (or ketamine/xylazine for t=0 h time point) and intubated. Aerosols of AONs (0.2 mg/kg) were administered into the trachea using a microsprayer (Penn-Century). Immediately after AON delivery (time=0 h) or at various time-points following delivery (time=2 h, 8 h, 16 h, 24 h, 48 h, 72 h and 144 h), mice were sacrificed using ketamine/xylazine. The lungs were removed, snap frozen in liquid nitrogen and stored at −80° C. until preparation of tissue homogenates. Lung tissues were homogenized in lysis buffer, centrifuged, and the resulting supernatants were snap frozen. A Hybridation-Enzyme-Linked-Immunosorbent-Assay (H-ELISA) method was used to perform quantification of each AON using specific probe sets. Standard curves were prepared for each AON using mouse lung tissue homogenate as a matrix.

### Rodent Model of Lung Inflammation

For all the animal studies the housing and care of mice (male C57Bl/6, Charles Rivers Laboratory) used in this study were provided according to protocols approved by Mispro Biotech's Institutional Animal Care and Use Committee, in conformity with Canadian Council on Animal Care (CCAC) guidelines. On days 1 to 5 and 8 to 12, groups of mice were anesthesized with isoflurane and intubated. Aerosols of vehicle (endotoxin-free PBS) or AONs or administered into the trachea using a microsprayer (Penn-Century). Bronchoalveolar lavages (BAL) were performed 24 h after the last treatment and differential cell counts were determined on at least 300 cells using standard morphological criteria. Lung tissues were harvested, formalin fixed and paraffin sections prepared. Histopathological evaluations were performed on Hematoxylin & Eosin stained slides.

The rat studies were performed at ITR Laboratories Canada (Bale d'Urfe, QC) in compliance with GLP regulations. Briefly, Male and female Sprague-Dawley rats received 14 consecutive doses of vehicle or 5 mg/kg of mg/kg of TOP004/TOP005 or TOP006/TOP007 (in a 1:1 w/w ratio in saline) administered daily as aerosols using a inhalation exposure system. The animals were examined 1-2 times daily for clinical symptoms including a qualitative assessment of food consumption, and body weight was measured weekly. Electrocardiographic (ECG) activity was recorded and ophthalmic examinations were conducted for animals pre-study and on Day 14.

One day after the last dose (Day 15), the rats (8/sex/group) were euthanized. Terminal procedures included complete gross necropsy examination, collection and preservation of approximately 40 tissues, and measurement of the weights of all major organs. Respiratory tract tissues (nasal cavity, nasopharynx, larynx, pharynx, trachea, bronchi, lungs including carina and bronchial lymph nodes) from all animals were examined by light microscopy, and all collected tissues was examined for all high dose and control group animals.

### Mouse Model of Lipopolysaccharide (LPS)-Induced Lung Inflammation

On day 1, groups of mice were anesthesized with isoflurane and intubated. Aerosols of vehicle (endotoxin-free PBS), AONs or controls were administered into the trachea using a microsprayer (Penn-Century). On day 1, other groups of mice were treated by gavage with vehicle (0.5% methylcellulose) or roflumilast (Rasayan). One hour after drug treatment, mice were exposed to saline or LPS (0.04 mg/mL) for 15 min in a plexiglass chamber. BAL were performed 3 h post-challenge and differential cell counts were determined on at least 300 cells using standard morphological criteria.

### Mouse Model of Cigarette Smoke-Induced Lung Inflammation

On days 1, 3, 6 and 8, or just 6 and 8 groups of mice were anesthesized with ketamine and xylazine (60 mg/kg and 12 mg/kg, i.p.) and intubated. Aerosols of vehicle (endotoxin-free PBS), AONs or controls were administered into the trachea using a microsprayer (Penn-Century). On days 6 to 9, other groups of mice were treated by gavage with vehicle (0.5% methylcellulose) or roflumilast (Rasayan). On days 6 to 9 (3 h after drug treatment), mice were nose-only exposed to the smoke of two 2R4f reference cigarettes (University of Kentucky) per day. Cigarette smoke was delivered to mice using a nose-only exposure system (Proto-Werx), at a rate of 1 puff (20 ml) per min. BAL were performed 18-24 h after the last smoke exposure and differential cell counts were determined on at least 300 cells using standard morphological criteria.

### Example 1

### Efficacy of AON Directed Against Human PDE7A

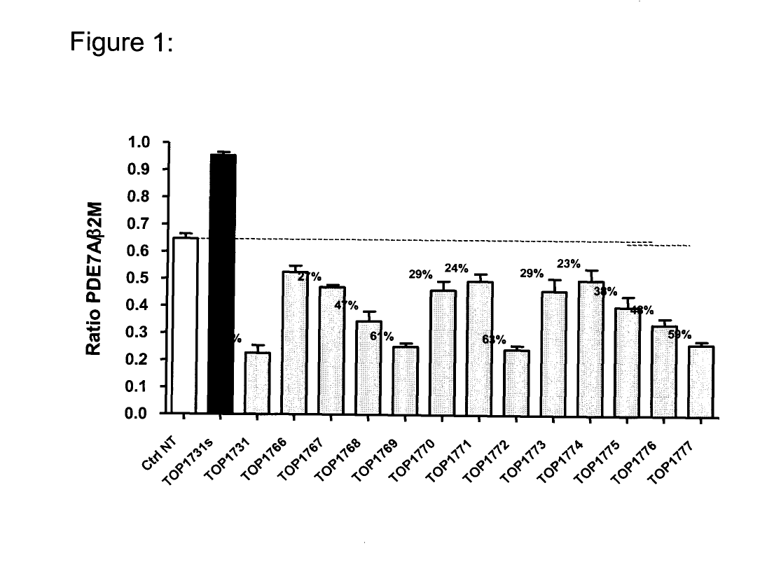

The sequences of the AON directed against the phosphodiesterase (PDE) isoform 7A are presented in Table 1. The potency of some selected phosphorothioate (PS) AON sequences (PS-DNA) is demonstrated in FIG. 1 which shows the reduction in gene expression following transfection with indicated AON sequence in the Flp-In T-Rex 293 cell line. The specificity of the selected AON sequences was assessed by comparing their efficacy at reducing PDE7A mRNA levels with the effect of a control sense sequence TOP1731s (SEQ. ID No. 128). AON comparative activity listed in Table 1 is expressed as the average percentage inhibition of PDE7A mRNA relative to untransfected controls (Ctrl NT).

### Example 2

### Efficacy of AON Directed Against Human PDE4D and PDE4B

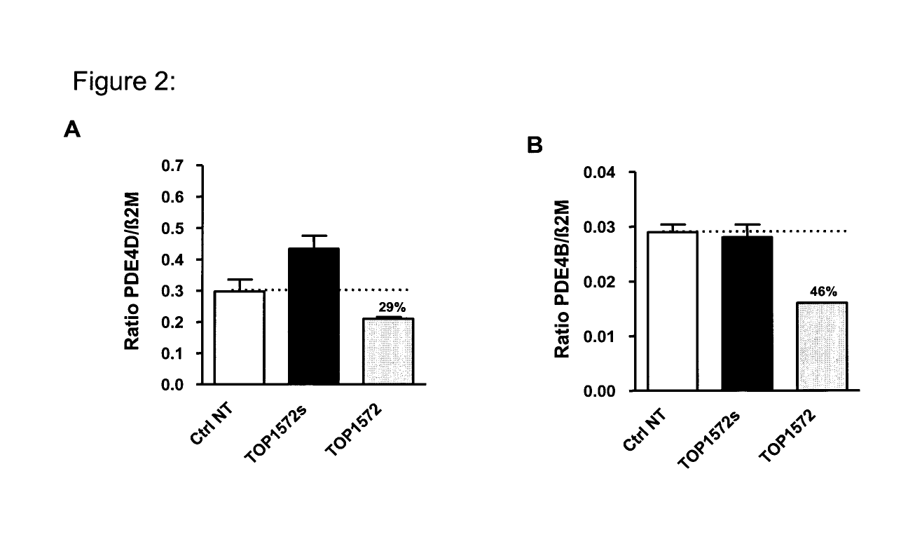

The sequences of the AON directed against the PDE isoforms 4D and 4B are presented in Table 2. The potency of some selected PS-DNA AON sequences is demonstrated in FIG. 2 which shows the reduction in gene expression following transfection with indicated AON sequences in the Flp-In T-Rex 293 cell line. The specificity of the selected AON sequences was assessed by comparing their efficacy at reducing PDE4D (FIG. 2A) and PDE4B (FIG. 2B) mRNA levels with the effect of a control sense sequence TOP1572s (SEQ. ID No. 131). AON comparative activity listed in Table 2 is expressed as the average percentage inhibition of PDE4D and PDE4B mRNA relative to untransfected controls.

### Example 3

### Efficacy of Human AON Sequences at Reducing PDE mRNA Levels Across Species

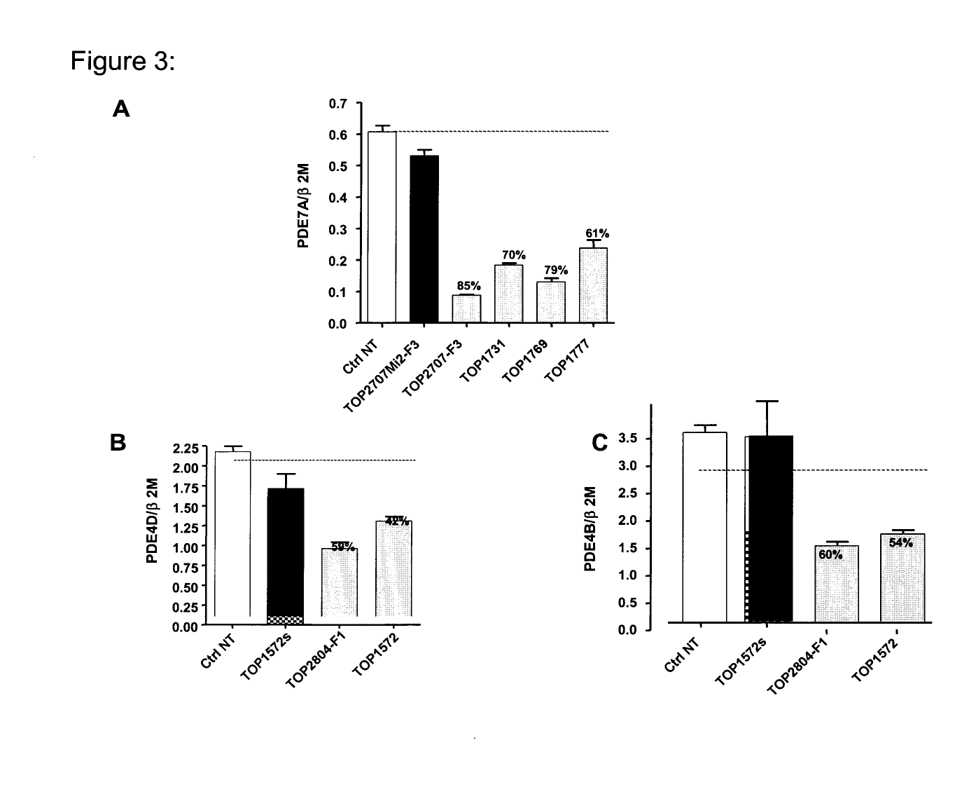

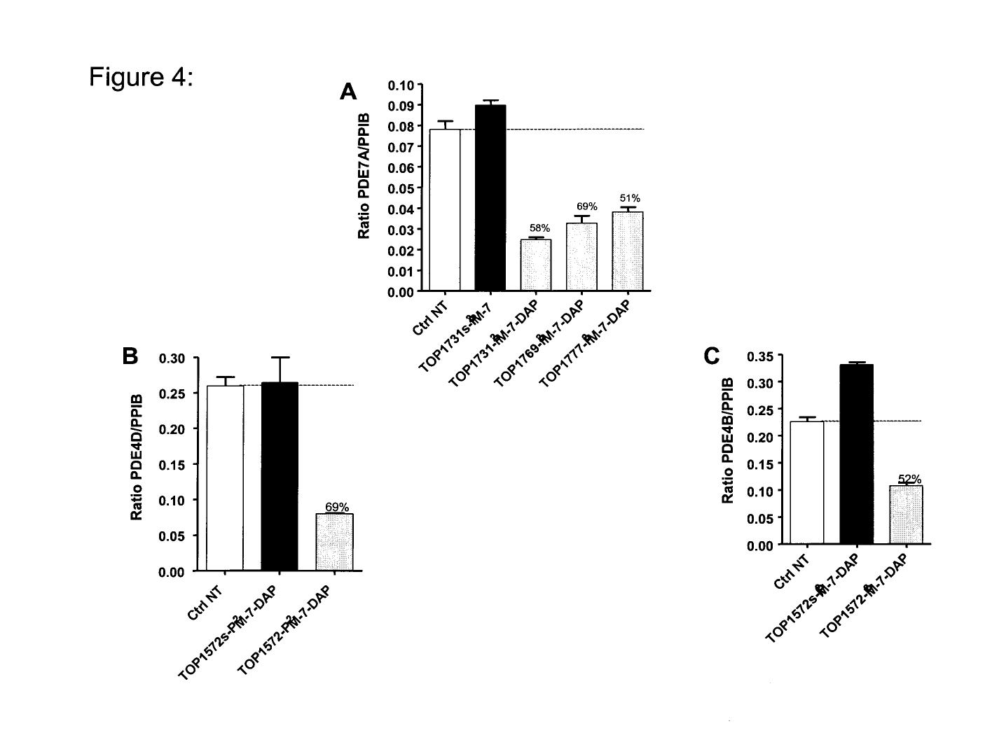

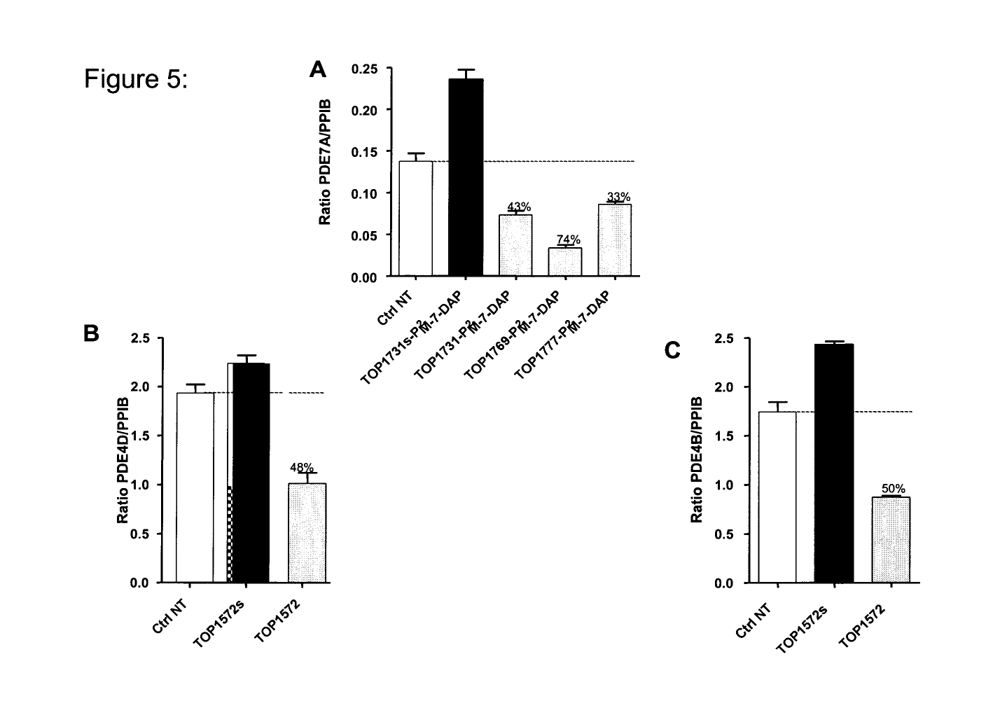

Some selected AON sequences targeting the human PDE genes were designed to maximize homology with the PDE gene sequence from other species, specifically the mouse, rat and monkey sequences. Table 3 describes the sequence homology and number of mismatch of selected AON sequences across these different species. FIG. 3 shows the specific reduction in PDE7A (FIG. 3A), PDE4D (FIG. 3B) and PDE4B (FIG. 3C) mRNA levels by some selected AON (PDE7A; TOP1731 (SEQ. ID No. 1), TOP1731-P²M-7-DAP (SEQ. ID No. 86), TOP1769 (SEQ. ID No. 33), TOP1769-P²M-7-DAP (SEQ. ID No. 95), TOP1777 (SEQ. ID No. 41), TOP1777-P²M-7-DAP (SEQ. ID No. 101), TOP2707-F3 (SEQ. ID No. 134); PDE4B/4D, TOP1572 (SEQ. ID No. 42), TOP1572-P²M-7-DAP (SEQ. ID No. 119), TOP2804-F1 (SEQ. ID No. 137)) following transfection for 24 h in NIH 3T3, a mouse cell line. The inhibitory activity of the selected AON sequences was also observed against the rat PDE mRNA in L2 cells (FIG. 4A-C) and the monkey PDE mRNA in CYNOM-K1 cells (FIG. 5A-C).

### Example 4

### AON with Both P²M and DAP Modifications Demonstrated Increased Efficacy for Target mRNA Knockdown

This example relates to the enhanced efficacy of specific AONs against various mRNA targets when P²M and DAP modifications are incorporated into the chemistry of the AON. Tables 4a-4-d and 10a-10b describe the compositions of AON modified with DAP residues and P²M linkages.

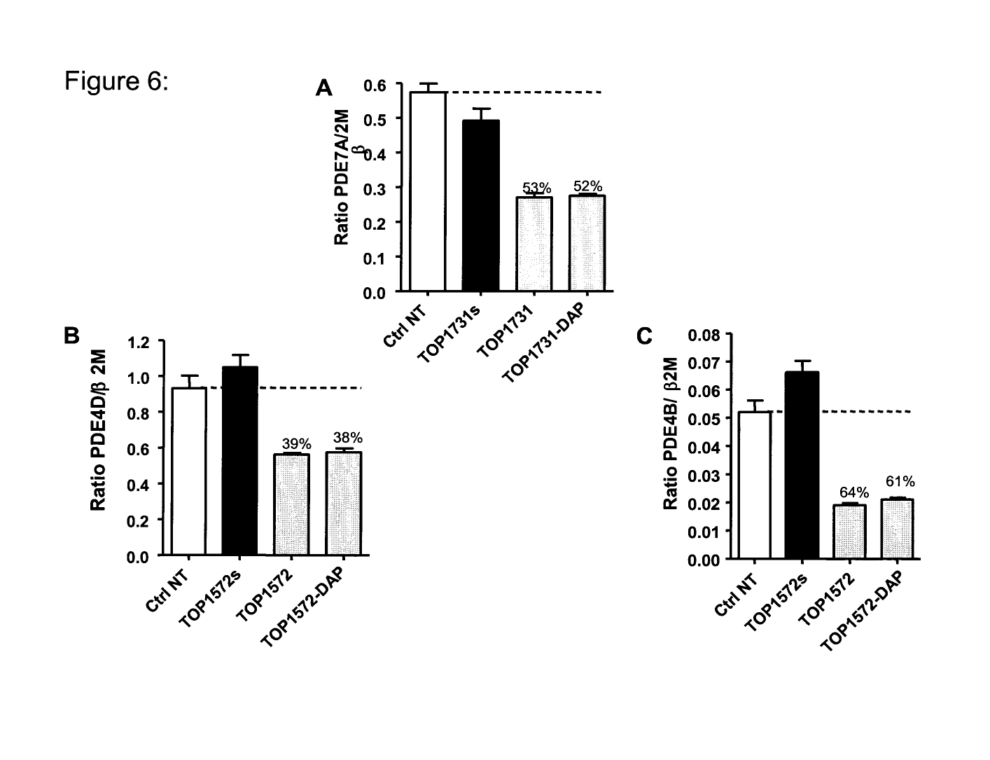

All adenosine bases of selected PS AON sequences were replaced with 2-amino-2′-deoxyadenosine bases (PS-DAP) and efficacy compared to unmodified PS-DNA AON sequences for their efficacy at reducing target mRNA expression in Flp-In T-Rex 293 cells transfected for 24 h. As shown in FIG. 6, the DAP modification didn't provide increased efficacy of TOP1731 (PS-DNA; TOP1731 (SEQ. ID No. 1), PS-DAP; TOP1731-DAP (SEQ. ID No. 74)) at reducing PDE7A (FIG. 6A) or TOP1572 (PS-DNA; TOP1572 (SEQ. ID No. 42), PS-DAP; TOP1572-DAP (SEQ. ID No. 107)) at reducing PDE4D (FIG. 6B) and PDE4B (FIG. 6C) compared to an unmodified version.

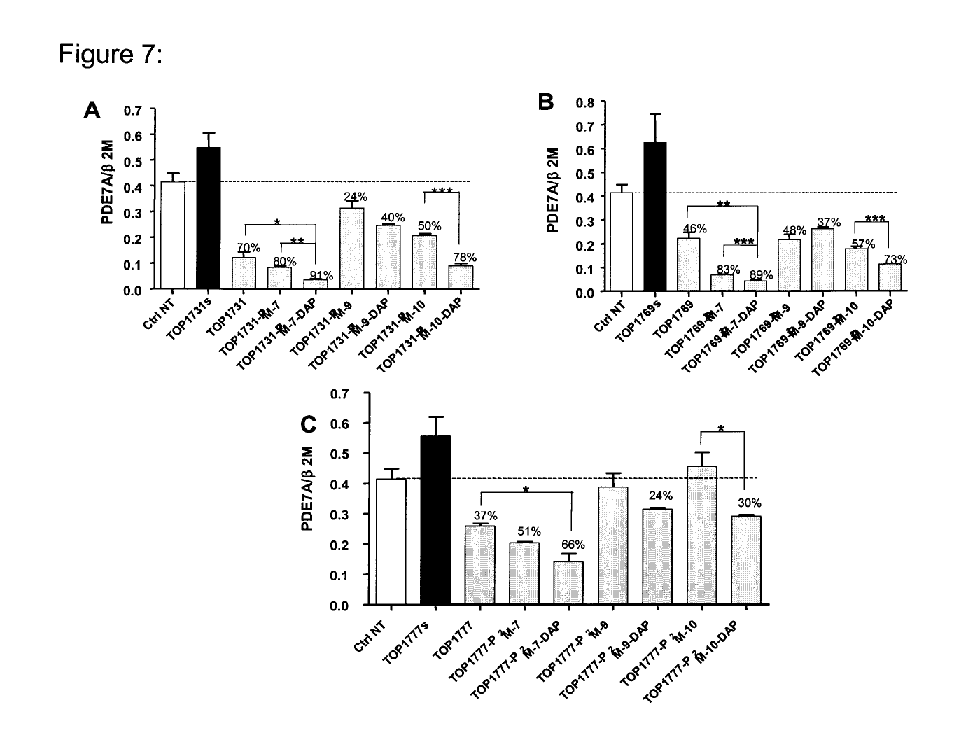

In FIG. 7, FIp-In T-Rex 293 cells were transfected for 24 h with 236 nM of a PDE7A-specific AON (A) TOP1731 (SEQ. ID No. 1), (B) TOP1769 (SEQ. ID No. 33) or (C) TOP1777 (SEQ. ID No. 41) either in phosphorothioate composition (PS-DNA) or containing P²M linkage modifications (P²M; TOP1731-P²M-7 (SEQ. ID No. 85), TOP1731-P²M-9 (SEQ. ID No. 89), TOP1731-P²M-10 (SEQ. ID No. 91), TOP1769-P²M-7 (SEQ. ID No. 94), TOP1769-P²M-9 (SEQ. ID No. 96), TOP1769-P²M-10 (SEQ. ID No. 98), TOP1777-P²M-7 (SEQ. ID No. 100), TOP1777-P²M-9 (SEQ. ID No. 102), TOP1777-P²M-10 (SEQ. ID No. 104)) as indicated in the legend provided in FIG. 7. Modification of TOP1731, TOP1769 or TOP1777 sequence with P²M linkages, (TOP1731-P²M-7 (SEQ. ID No. 85) TOP1769-P²M-7 (SEQ. ID No. 94) TOP1777-P²M-7 (SEQ. ID No. 100)) enhanced the efficacy of the AON to inhibit target gene expression, clearly showing an advantage of this modification for AON activity (FIG. 7A-C).

The DAP base modification was incorporated into AON sequences containing P²M linkage modifications. FIG. 7 shows that DAP base further enhanced the efficacy of P²M modified AON sequences (TOP1731-P²M-7-DAP (SEQ. ID No. 86), TOP1769-P²M-7-DAP (SEQ. ID No. 95), TOP1777-P²M-7-DAP (SEQ. ID No. 101)) to inhibit target gene expression, clearly showing a synergistic effect between the DAP modification and the P²M linkage modification for AON activity (FIG. 7A-C).

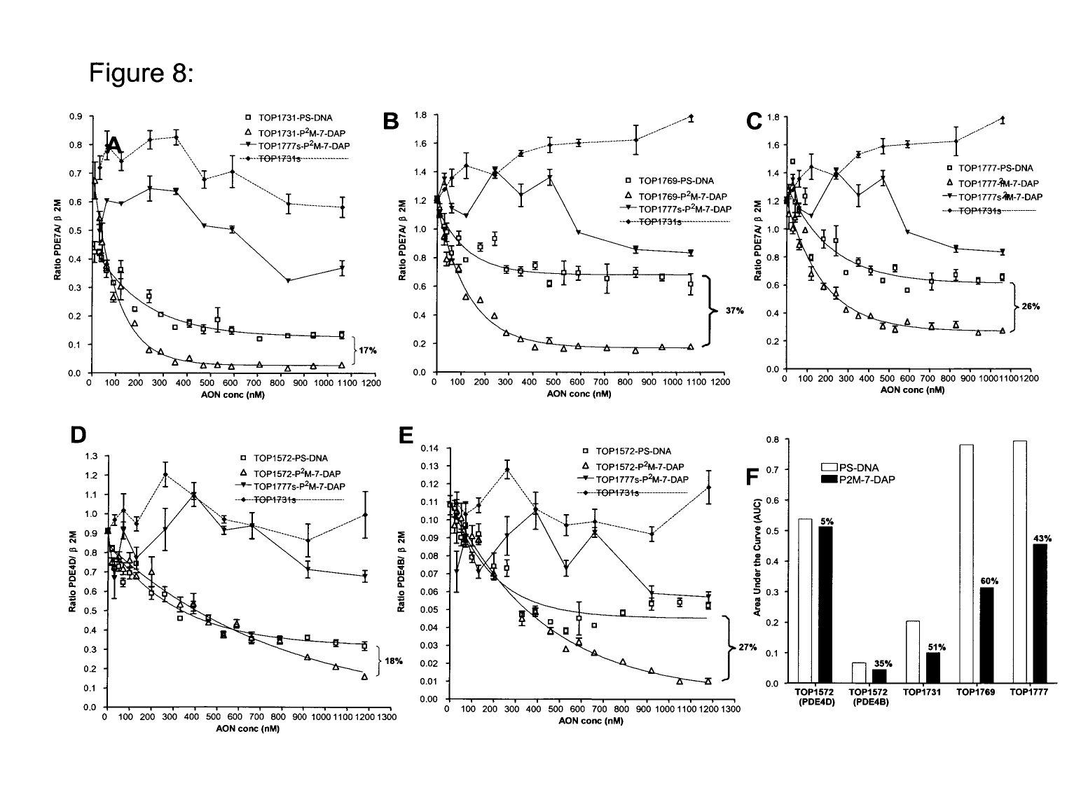

In FIG. 8, FIp-In T-Rex 293 cells were transfected for 24 h with increasing concentrations ranging between 15 and 1200 nM of PDE7A-specific AON (A) TOP1731 (SEQ. ID No. 1), (B) TOP1769 (SEQ. ID No. 33) or (C) TOP1777 (SEQ. ID No. 41) or PDE4B/4D-specific AON (D-E) TOP1572 (SEQ. ID No. 42) either in phosphorothioate composition (PS-DNA) or containing DAP base and P²M linkage modifications (P²M-DAP; TOP1731-P²M-7-DAP (SEQ. ID No. 86), TOP1769-P²M-7-DAP (SEQ. ID No. 95), TOP1777-P²M-7-DAP (SEQ. ID No. 101), TOP1572-P²M-7-DAP (SEQ. ID No. 119)) as indicated in the legend provided in FIG. 8. Results in FIG. 8 show that not only DAP base and P²M linkage modifications enhance the activity of the AON sequences compared to PS-DNA but also show that these modifications surprisingly increase the maximal target mRNA inhibition that the AON sequence can reach (FIG. 8A-F). The increased activity is surprising as a lower phosphorothioate/phosphodiester ratio is traditionally viewed to decrease stability (in fact, P²M-7-DAP indeed have a short half-life compared to PS-DNA, see Example 5 below). Furthermore, the advantageous increase in the maximal target mRNA inhibition is particularly surprising since changes in stability would likely affect activity, but not the maximal inhibition. As noted above in Example 4, modifying PS-DNA with DAP alone had little effect on inhibition.

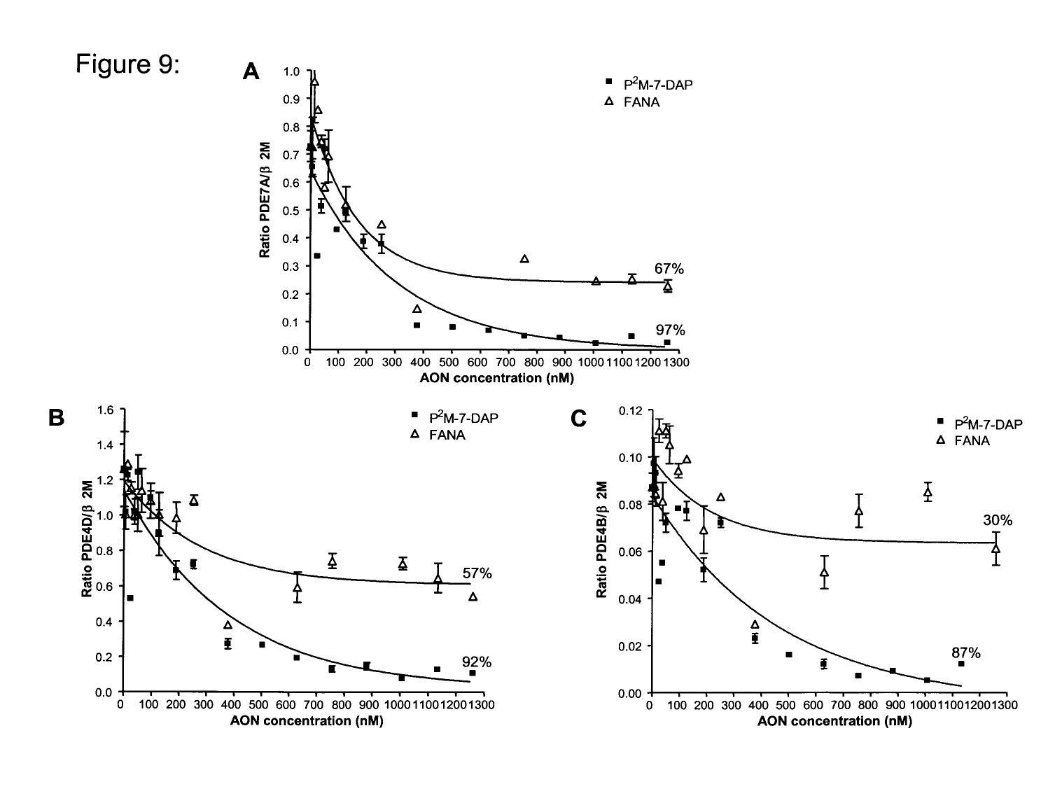

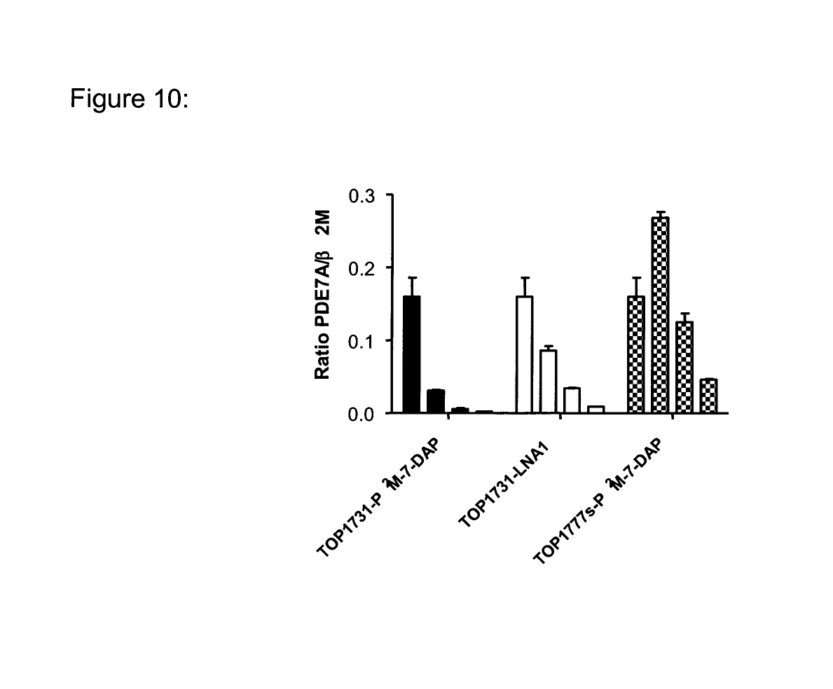

FIGS. 9 and 10 compare the activity of DAP base and P²M linkage modifications to other modifications used to improve the activity of AON. In FIG. 9, Flp-In T-Rex 293 cells were transfected for 24 h with increasing concentrations ranging between 6 and 1260 nM of a combination of either FANA-containing or P²M-DAP-containing versions of the PDE4B/4D AON sequence (TOP1572-P²M-7-DAP (SEQ. ID No. 119); TOP1572-F2 (SEQ. ID No. 106)) and the PDE7A AON sequence (TOP1731-P²M-7-DAP (SEQ. ID No. 86); TOP1731-F3 (SEQ. ID. No. 73)). Similarly to what was observed in the comparative analysis with unmodified PS-DNA AON sequences, results show that the P²M-DAP modifications have greater maximal target mRNA inhibition compared to FANA-containing AON sequences (FIG. 9A-C).

In FIG. 10, Flp-In T-Rex 293 cells were transfected for 24 h with increasing concentrations up to 713 nM of a locked nucleic acid (LNA)-containing or P²M-DAP-containing version of the PDE7A AON sequence (TOP1731-P²M-7-DAP (SEQ. ID No. 86); TOP1731-LNA1 (SEQ. ID. No. 93)). LNA AON have been shown to be very potent at inhibiting mRNA expression and have been describe as the most promising chemical modification (Kurreck J 2003, Eur. J. Biochem, 270:1628-1644; Jepsen and Wengel 2004, Curr. Opin. Drug Discov. Devel., 7:188-194). Results in FIG. 10 show that the DAP base and P²M linkage modifications enhance the activity of the AON sequences compared to LNA.

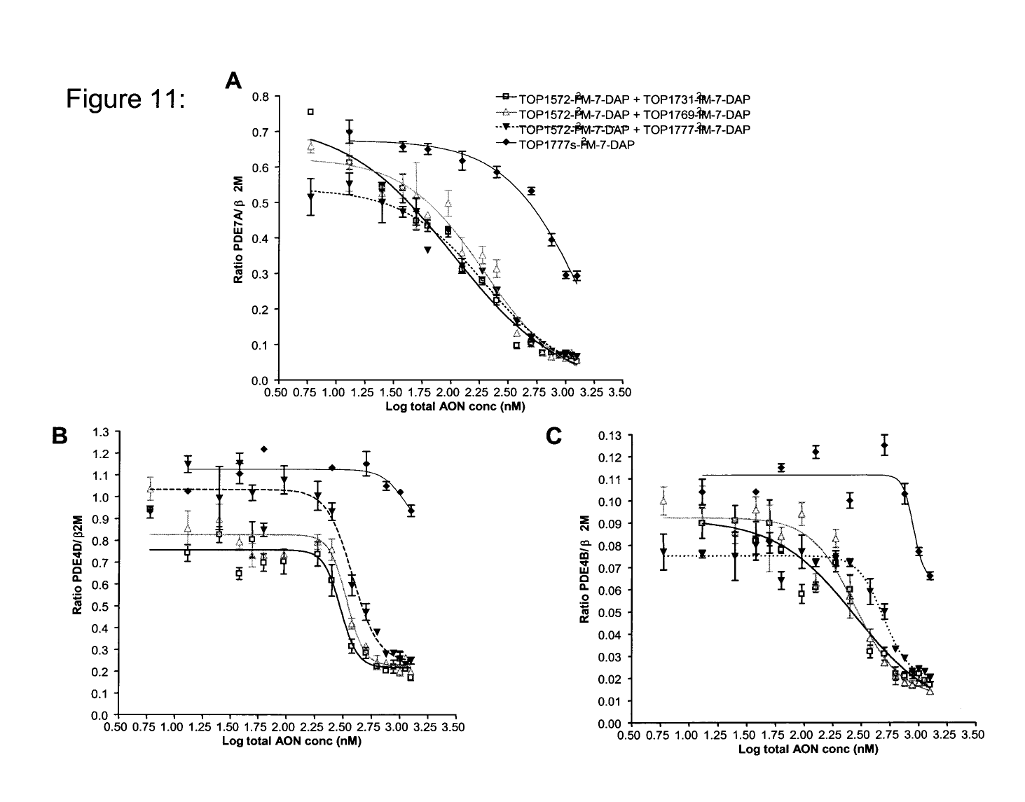

In FIG. 11, FIp-In T-Rex 293 cells were transfected for 24 h with increasing concentrations up to 1260 nM of combined PDE4B/4D and PDE7A P²M-7-DAP-containing AONs (PDE4B/4D: TOP1572-P²M-7-DAP (SEQ. ID No. 119); PDE7A: TOP1731-P²M-7-DAP (SEQ. ID No. 86); TOP1769-P²M-7-DAP (SEQ. ID No. 95), TOP1777-P²M-7-DAP (SEQ. ID No. 101) or the control sequence TOP1777s-P²M-7-DAP (SEQ. ID No. 132). Results in FIG. 10 show that all combinations of PDE4B/4D and PDE7A AON sequences provide potent target mRNA knock down.

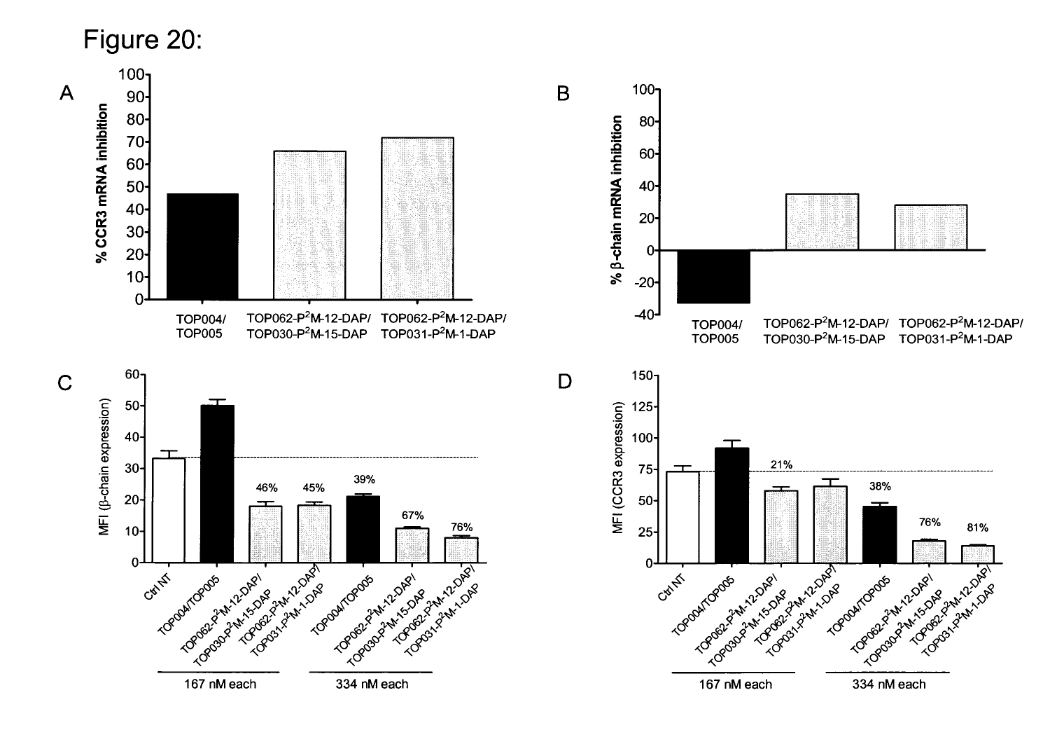

In FIG. 20, TF-1 cells were transfected for 24 h with increasing concentrations up to 1.34 μM of combined β-chain and CCR3 PS-DAP (TOP004 (SEQ. ID No. 157) and TOP005 (SEQ. ID No. 158)) or P²M-DAP-containing AONs (β-chain: TOP062-P²M-12-DAP (SEQ. ID No. 185); CCR3: TOP031-P²M-1-DAP (SEQ. ID No. 172); TOP030-P²M-15-DAP (SEQ. ID No. 170)). Results in FIG. 20 show that all combinations of P²M-DAP-containing AON AON sequences provide more potent target mRNA and protein knock down.

### Example 5

### AON Modified with P²M-DAP have Prolonged Duration of Action

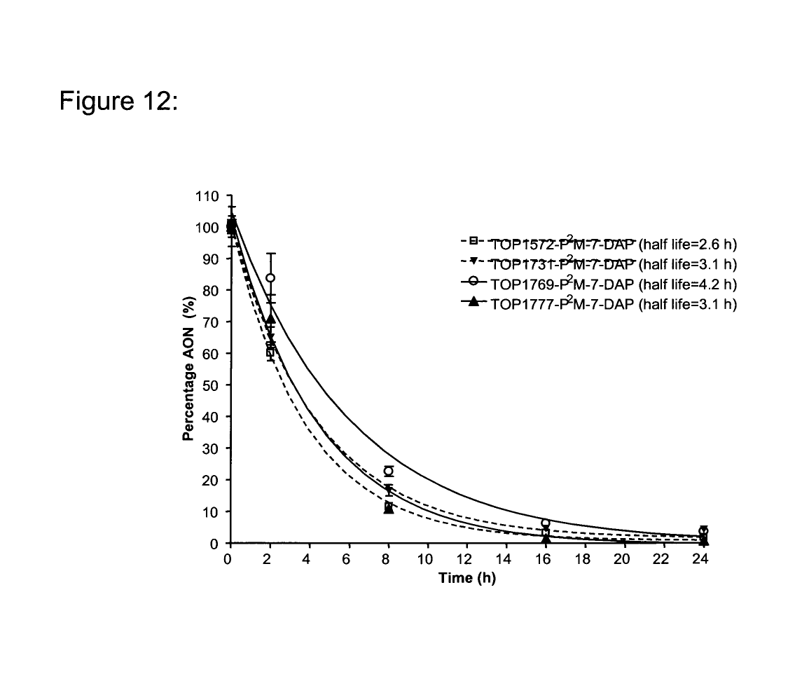

This example relates to the prolonged efficacy of P²M-DAP-containing AONs. P²M modifications are expected to reduce the stability of the AON due to the lower content of PS linkages, rendering it less resistant to nucleosidase digestion, which would shorten its activity in vitro and in vivo. The half-life of P²M-DAP-containing AONs was assessed in lung tissue. P²M-DAP-containing AONs were administered to mice via intra-tracheal aerosol and AON content measured at different time-points following administration. FIG. 12 shows that the tissue half-life the P²M-DAP-containing AONs are shorter than 5 h. The short half-life of the P²M-DAP-containing AONs is in contrast to the half-life measured for the unmodified PS-DNA AON sequences. TOP1731 (SEQ. ID No. 1) and TOP1572 (SEQ. ID No. 42) lung tissue half-life is 22 h and 26 h, respectively. AON sequences TOP1731-DAP (SEQ. ID No. 74) and TOP1572-DAP (SEQ. ID No. 107) with only the DAP-base modification have comparable half-life to the unmodified PS-DNA sequences.

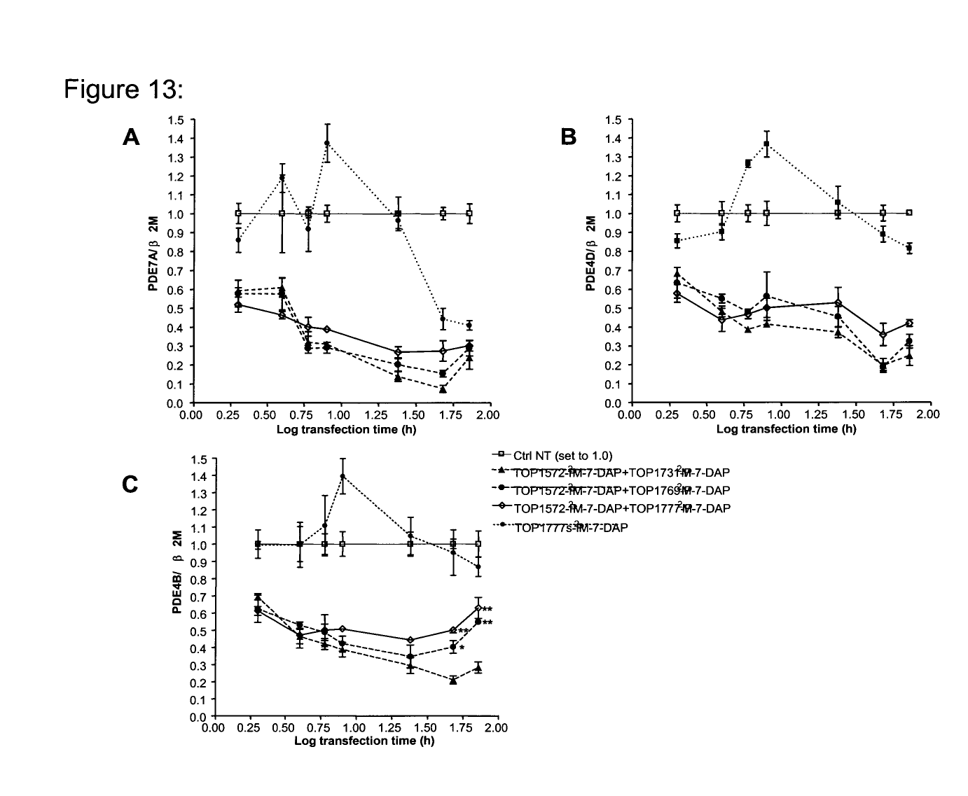

In FIG. 13, Flp-In T-Rex 293 cells were transfected for 24 h with 378 nM (total AON) of a combination of PDE4B/4D- and PDE7A-specific AON and target gene expression assessed over time sequences (PDE4B/4D; TOP1572-P²M-7-DAP (SEQ. ID No. 119), PDE7A; TOP1731-P²M-7-DAP (SEQ. ID No. 86), TOP1769-P²M-7-DAP (SEQ. ID No. 95), TOP1777-P²M-7-DAP (SEQ. ID No. 101)) or control sequence (TOP1777s-P²M-7-DAP (SEQ. ID No. 132).

Surprisingly, despite a relatively short half-life, the DAP base and P²M linkage modifications of the AON sequences have sustained inhibitory activity up to 72 h in vitro (FIG. 13A-C).

### Example 6

### Effect AON Modified with P²M-DAP Chemistry on Biological Function of Cells

This example relates to efficacy of P²M-DAP-containing AON on the biological function of different cell types, specifically the secretion of cytokines and chemokines. Cytokines and chemokines are important mediators of cell activation and recruitment while metalloproteases (MMP) are important modulators of tissue remodelling. Lung epithelial cells can play a role in the pathophysiology of inflammatory respiratory diseases through the secretion of chemokines that lead to the recruitment of immune cells, such as neutrophils and monocytes/macrophages, cytokines and metalloproteases. Interleukin-8 (IL-8/CXCL8) and monocyte chemoattractant protein-1 (MCP-1/CCL2) levels are increased in COPD patients (Szilasi M et al. Pathology Oncology Research. 2006, 12:52-60). The levels of IL-8 and MCP-1 may be involved in neutrophil and macrophage recruitment respectively. Similarly, MMPs may contribute to the pathogenesis of COPD (Baraldo et al., Chest 2007, 132:1733-40). MMP-2 and MMP-12 expression is increased in COPD patients.

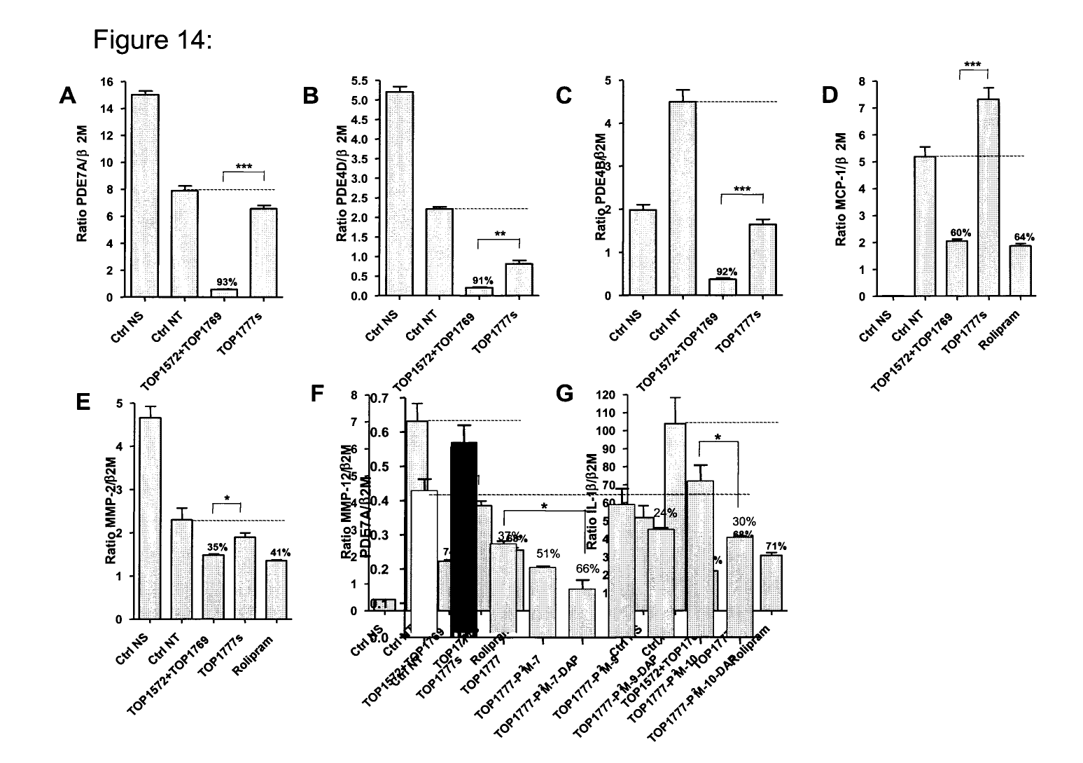

In FIG. 14, normal human bronchial epithelial (NHBE) cells were transfected for 20 h with 250 nM total of a combination of PDE4B/4D- and PDE7A-specific AON sequences (TOP1572-P²M-7-DAP (SEQ. ID No. 119), TOP1769-P²M-7-DAP (SEQ. ID No. 95) or control sequence (TOP1777s-P²M-7-DAP (SEQ. ID No. 132) and then were stimulated with 10 ng/mL cytomix (TNF-α+IL-1β+IFN-γ) for 4 h. Results show that gene expression of (D) MCP-1, (E) MMP-2, (F) MMP-12 and (G) IL-1β is increased following stimulation of NHBE cells. The combination of P²M-DAP modified PDE4B/4D- and PDE7A-specific AON sequences (TOP1572-P²M-7-DAP (SEQ. ID No. 119), TOP1769-P²M-7-DAP (SEQ. ID No. 95) can limit their respective target gene expression (FIG. 14A-C) and block the induction of gene expression (FIG. 14D-G). The activity of the AON on the expression of MCP-1, MMP-2, MMP-12 and IL-1β is markedly greater than that of Rolipram, a small molecule PDE4 inhibitor, considering that the concentration of Rolipram used was >2-log higher than that of the AON.

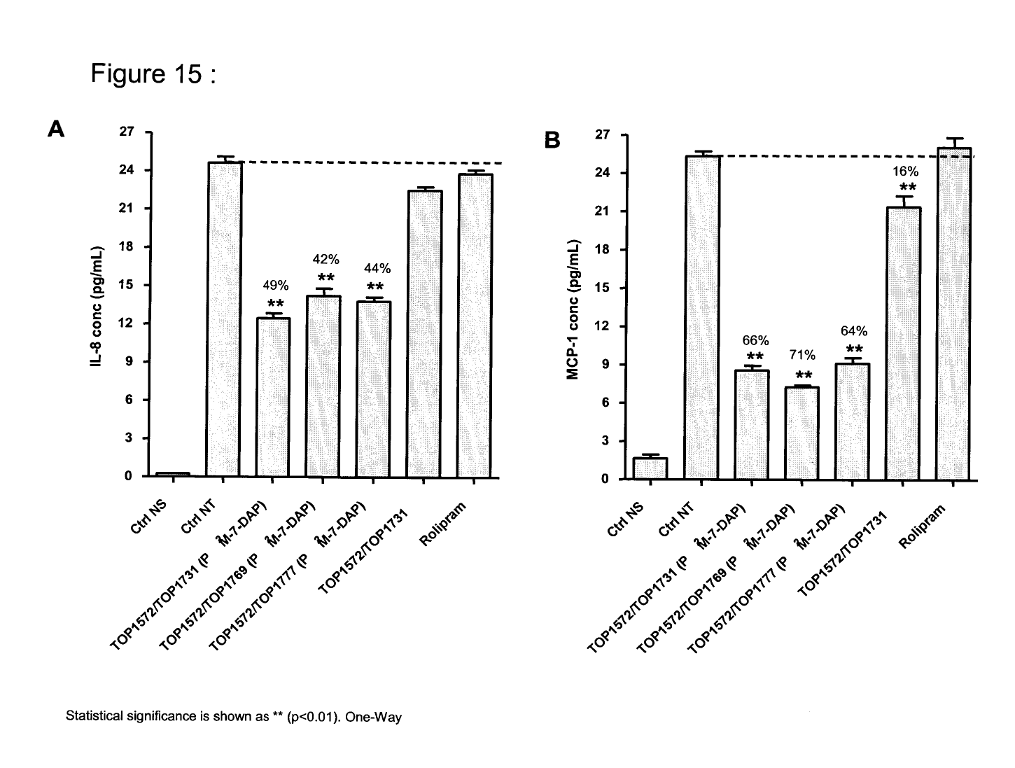

In FIG. 15, lung epithelial A549 cells were transfected for 20 h with 238 nM total of a combination of PDE4B/4D- and PDE7A-specific AON sequences (PDE4B/4D; TOP1572 (SEQ. ID No. 42), TOP1572-P²M-7-DAP (SEQ. ID No. 119), PDE7A; TOP1731 (SEQ. ID No. 1), TOP1731-P²M-7-DAP (SEQ. ID No. 86), TOP1769-P²M-7-DAP (SEQ. ID No. 95), TOP1777-P²M-7-DAP (SEQ. ID No. 101)) and were stimulated with IL-1β for 6 h to induce protein secretion of IL-8/CXCL-8 and MCP-1/CCL2. The DAP base and P²M linkage modifications of the AON enhanced the activity of the AON to limit the secretion of the pro-inflammatory chemokines IL-8/CXCL-8 and MCP-1/CCL2 compared to the unmodified (PS-DNA) AON.

### Example 8

### In Vitro and In Vivo Response to P²M-DAP Modified AONs

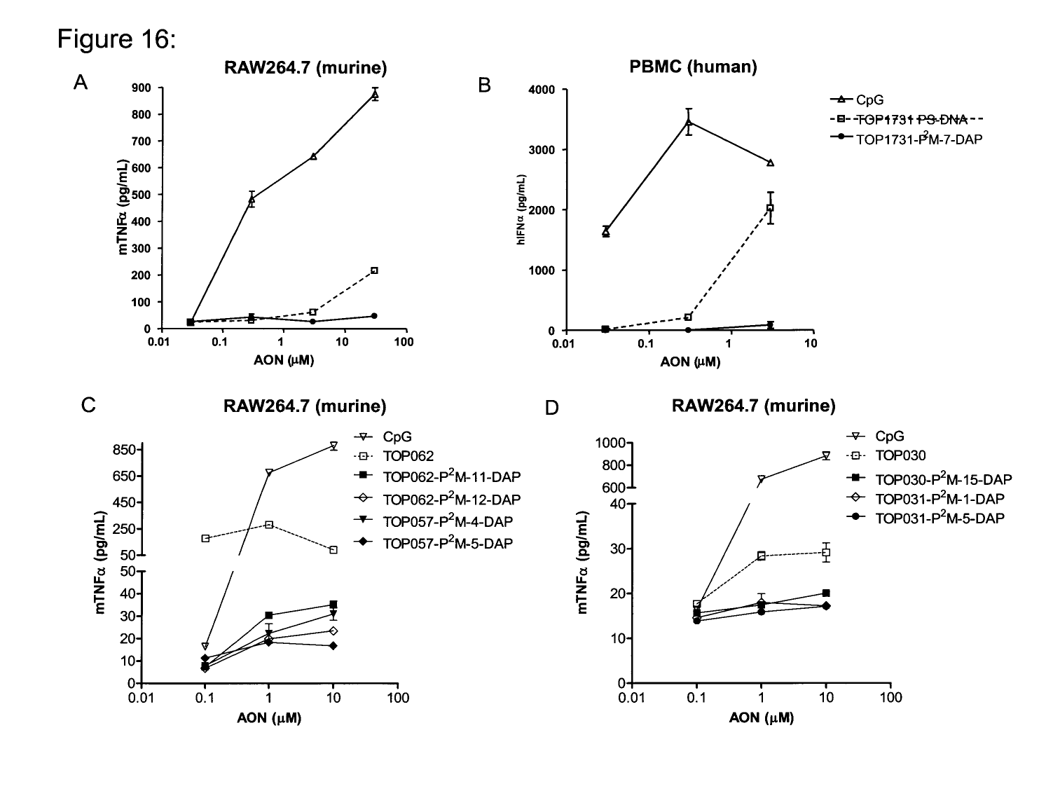

This example relates to the relative reduction of the immune and inflammatory response of P²M-DAP modified AONs in vitro as well as in vivo following chronic dosing administration to mice. FIGS. 16 (A, B, C and D) shows the in vitro response of mouse macrophages (RAW 264.7 cells) and human PBMC when exposed for 24 h to either the unmodified PS-DNA sequence TOP1731 (SEQ. ID No. 1), TOP062 (SEQ. ID No. 179) and TOP030 (SEQ. ID No. 161) or the P²M-DAP-modified sequence TOP1731-P²M-7-DAP (SEQ. ID No. 86), TOP062-P²M-11-DAP (SEQ. ID No. 184), TOP062-P²M-12-DAP (SEQ. ID No. 185), TOP057-P²M-4-DAP (SEQ. ID No. 193), TOP057-P²M-5-DAP (SEQ. ID No. 194), TOP030-P²M-15-DAP (SEQ. ID No. 170), TOP031-P²M-1-DAP (SEQ. ID No. 172) or TOP031-P²M-5-DAP (SEQ. ID No. 176). A known immunostimulatory sequence (CpG (SEQ ID No. 133)) was used as positive control. Results indicate that the PS-DNA sequence induced a dose-dependent secretion of TNF-α and IFN-α in RAW 264.7 and PBMC, respectively, while the incorporation of the P²M-DAP modifications completely abrogated this immunostimulatory effect. This result is surprising as the broader activity of known immunostimulatory sequences, such as the CpG sequence (SEQ. ID No. 133) used in this study as positive control, is known to be dependent on the presence of mixed PS/PO linkages (Krieg A. 2002 Annu. Rev. Immunol, 20:709-760).

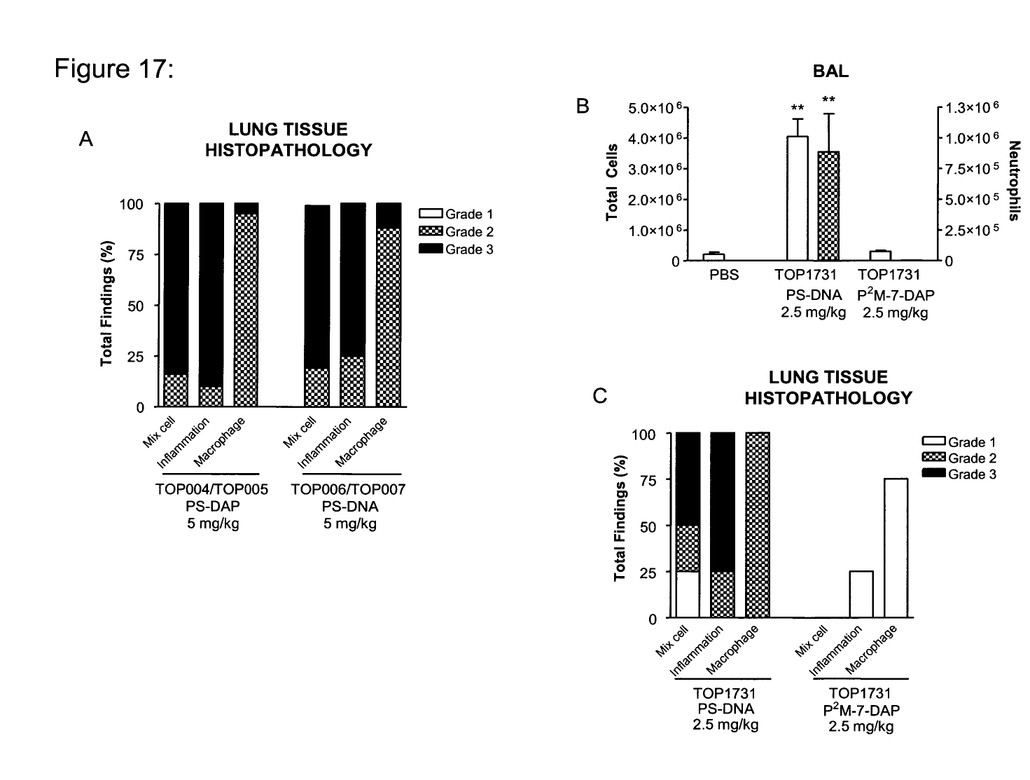

The response of unmodified PS-DNA and DAP-modified AON sequences was also assessed in vivo following administration by inhalation of 14 consecutive doses of 5 mg/kg to rats (saline was used as control). Lung tissue histolopathology was assessed 24 h following the last AON treatment. FIG. 17A shows the histopathological changes observed in the animals following treatment with the selected unmodified or DAP-modified AON sequences. Results indicate that animal treated with the unmodified or the DAP-modified AON sequences had comparable type of findings, incidence of findings and severity of findings clearly showing no benefit of the DAP base modification alone for the tolerability of the AON.

The response of P²M-DAP-modified AON sequences intra-tracheal administration of 10 doses of 2.5 mg/kg to mice (PBS was used as control). Differential cell counts of bronchoalveolar lavage (BAL) and lung tissue histolopathology were assessed 24 h following the last AON treatment. Table 8 summarizes the influx of inflammatory cells in BAL and the histopathological assessment of the selected P²M-DAP and unmodified AON sequences (PS-DNA). As shown in FIG. 17B, results indicated that animals receiving the P²M-DAP-modified AON (TOP1731-P²M-7-DAP (SEQ. ID No. 86)) had a lower influx of total cells, represented by a decrease in macrophage, lymphocytes and neutrophils, compared to the unmodified PS-DNA sequence TOP1731 (SEQ ID No. 1). Mice treated with the P²M-DAP-modified AON also had a lower incidence of lung tissue findings and the findings observed in these animals were of lesser severity (Grade 1 reflects a minimal severity while Grade 4 reflects severe severity) compared to the unmodified PS-DNA AON (FIG. 17C) clearly showing a benefit of the DAP base and P²M linkage modifications for the tolerability of the AON.

Table 8 also summarizes the efficacy/toxicity benefit ratio calculated for the selected P²M-DAP-containing AON sequences. This ratio takes into account the activity (referred to efficacy index or as example here the maximal target knockdown obtained at a given concentration) of the AON vs its relative toxicity (here referred to lung toxicity index or the sum of all the findings and their severity grade). Results in Table 8 show very low ratio values for the unmodified AON sequences (PS-DNA) and higher ratio values (>0.25) for the P²M-DAP-containing AON sequences.

### Example 9

### P²M-DAP-modified AON Efficacy in a Mouse Model of LPS-Induced Airway Inflammation

This example relates to the efficacy of AON targeting the PDE4B/4D and PDE7A when P²M-DAP modifications are incorporated into the chemistry of the AON in an in vivo model of acute lung inflammation in mice. Tables 4c, 4d and 6 describe the compositions of AONs targeting the human or mouse PDE4B/4D and PDE7A and modifications with P²M-DAP.

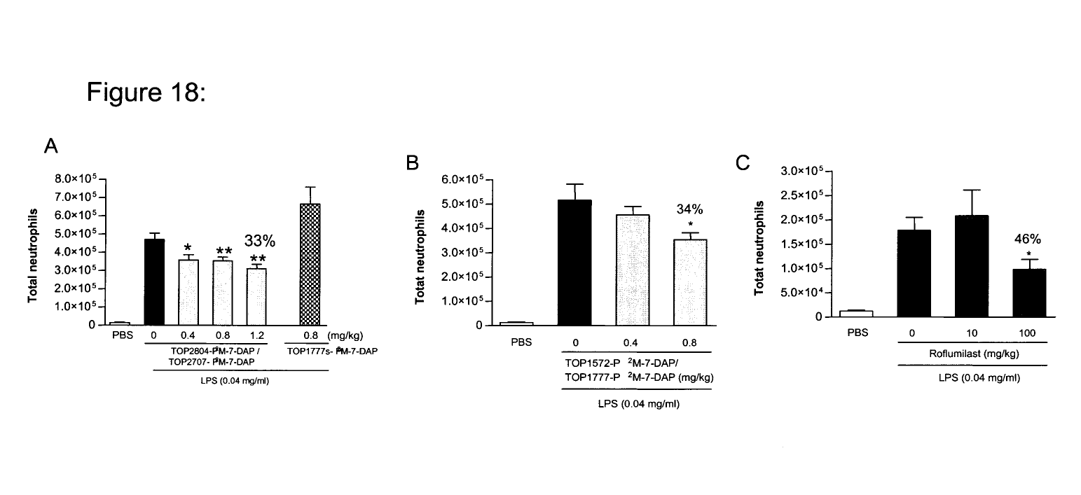

The activity of P²M-DAP-modified AONs targeting the PDE4B/4D and PDE7A was demonstrated in an in vivo model of LPS-induced acute lung inflammation model in mice. In this model of inflammation, mice are exposed to LPS resulting in a marked influx of neutrophils in the lungs of the animals (FIG. 18). Neutrophils are a key cell underlying the inflammatory response in many inflammatory diseases such as in COPD. When mice were treated prior to LPS-exposure with a combination (ratio 1:1 w/w) of one modified AON targeting PDE4B/4D (TOP572-P²M-7-DAP (SEQ. ID No.,) or TOP2804-P²M-7-DAP (SEQ. ID No. 138)) and one modified AON targeting PDE7A (TOP1777-P²M-7-DAP (SEQ. ID No. 101) or TOP2707-P²M-7-DAP (SEQ. ID No. 136)), significant reduction of the LPS-induced neutrophil influx was observed (FIG. 18A-B). However, when LPS-exposed mice were treated with a control sequence (TOP1777s-P²M-7-DAP (SEQ. ID No. 132), the neutrophil influx to the lung was not reduced (FIG. 18A). In mice exposed to LPS but treated with roflumilast, a small molecule inhibitor of PDE4, a dose of 100 mg/kg was required in order to reduce neutrophil influx by a level comparable to that obtained with the AON (FIG. 18C).

### Example 10

### P²M-DAP-Modified AON Efficacy in a Mouse Model of Cigarette Smoke-Induced Airway Inflammation

This example relates to the efficacy of AON targeting the PDE4B/4D and PDE7A when P²M-DAP modifications are incorporated into the chemistry of the AON in an in vivo model of cigarette smoke-induced lung inflammation in mice. Tables 4c, 4d and 6 describe the compositions of AONs targeting the human or mouse PDE4B/4D and PDE7A and modifications with P²M-DAP.

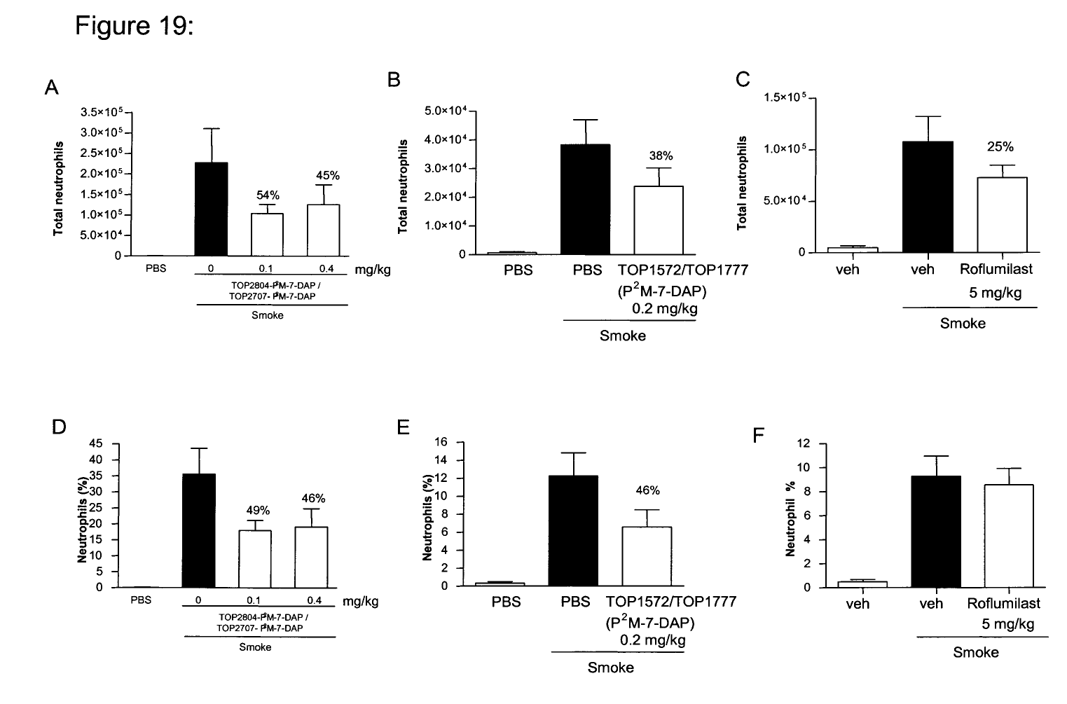

The activity of P²M-DAP-modified AONs targeting the PDE4B/4D and PDE7A was also demonstrated in an in vivo model of cigarette smoke-induced lung inflammation model in mice. In this model of inflammation, mice are exposed to cigarette smoke for 4 consecutive days resulting in a marked influx of neutrophils in the lungs of animals (FIG. 19). When mice were treated every other day prior to smoke exposure with a combination (ratio 1:1 w/w) of one P²M-DAP-modified AON targeting PDE4B/4D (TOP572-P²M-7-DAP (SEQ ID No:) and one P²M-DAP-modified AON targeting PDE7A (TOP1777-P²M-7-DAP (SEQ ID No. 101), a reduction of the total (FIGS. 19A and B) and the percentage (FIGS. 19D and E) neutrophil influx was observed. In mice exposed to cigarette smoke but treated with roflumilast, a small molecule inhibitor of PDE4, a dose of 5 mg/kg was required in order to reduce total neutrophil influx (FIG. 19C) but with no effect of the percentage of neutrophils (FIG. 19F).

The long acting nature of the P²M-DAP-modified AONs was again demonstrated, as they were administered every 2 days compared to roflumilast, which was administered daily.

Although preferred embodiments of the invention have been described herein, it will be understood by those skilled in the art that variations may be made thereto without departing from the spirit of the invention or the scope of the appended claims. All references cited herein are incorporated by reference.

TABLE 1

| | Seq | | | | % PDETA |
|---|---|---|---|---|---|
| AON ID | ID No. | Length | AON sequence 5′-3′ | Target sita^{a} | Inhibition^{b} |
| TOP1731 | 1 | 21 | TCATGAGTGGCAGCTGCAATT | 805 | >50% |
| TOP1738 | 2 | 19 | CAGTCTGATCAGAACTGTC | 227 | 20-50% |
| TOP1739 | 3 | 20 | CATCTCCTAGCATACGAATG | 253 | 20-50% |
| TOP1740 | 4 | 19 | GGCTCCTTACACGTACATC | 269 | >50% |
| TOP1741 | 5 | 20 | GACAGACACTTCAATTTCAG | 357 | 20-50% |
| TOP1742 | 6 | 19 | GCTGGAAACTTAGTAGCCT | 395 | <20% |
| TOP1743 | 7 | 18 | GTGAAGATCTAAGATATC | 414 | <20% |
| TOP1744 | 8 | 19 | TGAAACCGCAGTACCACGA | 339 | >50% |
| TOP1745 | 9 | 19 | AGGTTAAGCTTACTAGACT | 572 | 20-50% |
| TOP1746 | 10 | 19 | TGGAAGTACTCAATTAATC | 612 | <20% |
| TOP1747 | 11 | 20 | CTACGAAGTTTCATCATATC | 635 | 20-50% |
| TOP1748 | 12 | 19 | GGACTGCGTTATGGTAAGG | 695 | 20-50% |
| TOP1749 | 13 | 19 | CATGGCCTGAGTAACATCC | 721 | 20-50% |
| TOP1750 | 14 | 19 | CAGAATTGGCAAGCTTAGG | 758 | 20-50% |
| TOP1751 | 15 | 19 | ATGATCCAGATCATGAGTG | 817 | 20-50% |
| TOP1752 | 16 | 19 | GTTGCCAAGTAATGGTTAG | 867 | 20-50% |
| TOP1753 | 17 | 19 | CAGTACTGAGGTATTCTTG | 892 | 20-50% |
| TOP1754 | 18 | 18 | CACTGCAGATCTCCAGTG | 920 | 20-50% |
| TOP1755 | 19 | 19 | GAATAAGCCTGATTCTCTC | 946 | <20% |
| TOP1756 | 20 | 20 | GAGCACCTATCTGTGTCTCC | 997 | 20-50% |
| TOP1757 | 21 | 20 | GTGGCTAGTATCAGAGCACC | 1010 | 20-50% |
| TOP1758 | 22 | 19 | GACTGATGTCTGTGGCTAG | 1022 | <20% |
| TOP1759 | 23 | 19 | GTGTCTGGTGTCTTCTAGG | 1096 | <20% |
| TOP1760 | 24 | 20 | CCACGTCCGACATGGGTTAC | 1158 | <20% |
| TOP1761 | 25 | 19 | TCTATATCTCCTTGATGGA | 1221 | <20% |
| TOP1762 | 26 | 19 | TAGATTCAGTGTGACGATC | 1274 | <20% |
| TOP1763 | 27 | 19 | ACCAATCTGGATGTTGGCA | 1294 | <20% |
| TOP1764 | 28 | 19 | CTGGATGATCCAGATCATG | 821 | <20% |
| TOP1765 | 29 | 18 | GGCTTGTCCATTATAATC | 482 | <20% |
| TOP1766 | 30 | 20 | ACATCTCCTAGCATACGAAT | 254 | <20% |
| TOP1767 | 31 | 21 | AAGGTTAAGCTTACTAGACTA | 571 | 20-50% |
| TOP1768 | 32 | 21 | GTGCATGGCCTGAGTAACATC | 722 | 20-50% |
| TOP1769 | 33 | 21 | ACAGTGCATGGCCTGAGTAAC | 725 | >50% |
| TOP1770 | 34 | 21 | CAGAATTGGCAAGCTTAGGTT | 756 | 20-50% |
| TOP1771 | 35 | 21 | TGGCAAGCTTAGGTTCCTTTA | 750 | 20-50% |
| TOP1772 | 36 | 20 | TGATCCAGATCATGAGTGGC | 815 | >50% |
| TOP1773 | 37 | 21 | TAAAGTTGCCAAGTAATGGTT | 869 | 20-50% |
| TOP1774 | 38 | 21 | ACTGAGGTATTCTTGTATAAA | 886 | 20-50% |
| TOP1775 | 39 | 20 | GCAGATCTCCAGTGGTGATT | 914 | 20-50% |
| TOP1776 | 40 | 21 | CATACACTTGGCTTGTCCATT | 488 | 20-50% |
| TOP1777 | 41 | 21 | TAAGTAACAGTGCATGGCCTG | 731 | >50% |

^{a}Target site on PDE7A1 mRNA sequence (accession # NM_002603)
^{b}Percentage of PDE7A mRNA expression in Flp-In T-Rex 293 cells (260 nM)

TABLE 2

| | Seq | | | Taget site^{a} | | % Inhibition^{b} | |
|---|---|---|---|---|---|---|---|
| AON ID | ID No. | Length | AON sequence 5′-3′ | PDE4D | PDE4B | PDE4D | PDE4B |
| TOP1572 | 42 | 19 | GGTTGCTCAGITCTGCACA | 1875 | 2024 | >50% | >50% |
| TOP1575 | 43 | 16 | AICATCCTTTTIAACT | 937 | 1086 | <20% | <20% |
| TOP1576 | 44 | 16 | AGICCCCATTTGTTCA | 1225 | 1374 | <20% | <20% |
| TOP1577 | 45 | 17 | GCCACITCAGCITGGTA | 1386 | 1535 | <20% | <20% |
| TOP1578 | 46 | 17 | ACCATGTCIATIACCAT | 1716 | 1865 | <20% | <20% |
| TOP1579 | 47 | 16 | TCATITGTTTIGACAT | 1746 | 1895 | <20% | <20% |
| TOP1580 | 48 | 17 | GAICTIGTCACTTTCTT | 1797 | 1946 | <20% | <20% |
| TOP1581 | 49 | 17 | GCACAGTGCACCATITT | 1863 | 2012 | 20-50% | <20% |
| TOP1582 | 50 | 18 | GCTCAGITCIGCACAGTG | 1872 | 2021 | <20% | <20% |
| TOP1583 | 51 | 17 | TCACACATIGGGCTIAT | 1989 | 2138 | <20% | <20% |
| TOP1584 | 52 | 17 | TTITCACACATIGGGCT | 1992 | 2141 | <20% | <20% |
| TOP1585 | 53 | 17 | GCCATTITCCACATCAA | 475 | 1037 | <20% | <20% |
| TOP1586 | 54 | 17 | GGATCCAIIGGACTCCG | 504 | 1526 | <20% | <20% |
| TOP1587 | 55 | 20 | GTCATAITCGCTGTCIGATC | 601 | 2219 | <20% | <20% |
| TOP1588 | 56 | 18 | GACCTGIGCAAAIGGAGT | 690 | 2022 | <20% | <20% |
| TOP1589 | 57 | 17 | GCCATCTCACTGACIGA | 912 | 1061 | <20% | <20% |
| TOP1590 | 58 | 17 | CTTTTIAACTTGTTIGA | 930 | 1079 | <20% | <20% |
| TOP1591 | 59 | 17 | TCTAAGAAIGTITTTGA | 1017 | 1166 | <20% | <20% |
| TOP1592 | 60 | 17 | TGCTTITCTAAGAAIGT | 1023 | 1172 | <20% | <20% |
| TOP1593 | 61 | 17 | TTCAGTITTIACTCCAA | 1174 | 1323 | <20% | <20% |
| TOP1594 | 62 | 17 | TGGTCTTCIAIAGTCAT | 1368 | 1517 | <20% | <20% |
| TOP1595 | 63 | 17 | ATCTCCAAATCTGTIAA | 1482 | 1631 | <20% | <20% |
| TOP1596 | 64 | 17 | AGIATCTCCAAATCTGT | 1485 | 1634 | <20% | <20% |
| TOP1597 | 65 | 17 | GGATGATCIACITCATG | 1527 | 1676 | <20% | <20% |
| TOP1598 | 66 | 17 | ACICCAGGATGATCIAC | 1533 | 1682 | 20-50% | <20% |
| TOP1599 | 67 | 17 | GCAAGTTCIGAITTTGT | 1572 | 1721 | <20% | <20% |
| TOP1600 | 68 | 18 | CTTIGTIGGGTTGCTCAG | 1884 | 2033 | <20% | 20-50% |
| TOP1601 | 69 | 17 | TCACATIGGGCTIAT | 1989 | 2138 | <20% | <20% |
| TOP1602 | 70 | 17 | GGATGIACAATITAGTC | 2049 | 2198 | <20% | 20-50% |
| TOP1603 | 71 | 17 | GAIGCCATCTCACTGAC | 915 | 1064 | <20% | 20-50% |
| TOP1604 | 72 | 17 | GTAATGGTCTTCIAIAG | 1372 | 1521 | <20% | <20% |

^{a}Target site on PDE4B1 (accession #NM_001037341) or PDE4D1 (accession # NM_001104631) mRNA sequence
^{b}Percentage of PDE4B or PDE4D mRNA expression in Flp-In T-Rex 293 cells (260 nM)
)

TABLE 3

| | | | Number of mismatch(es) | | | |
|---|---|---|---|---|---|---|
| ID | Target(s) | *AON sequence (5′-3′) | Human | Mouse | Rat | Monkey |
| TOP1731 | PDE7A | tcatgagtggcagctgcaatt | 0 | 2 | 0 | 0 |
| TOP1769 | | acagtgcatggcctgagtaac | 0 | 0 | 1 | 0 |
| TOP1777 | | taagtaacagtgcatggcctg | 0 | 0 | 0 | 0 |
| TOP1572 | PDE4B/4D | ggttgctcagitctgcaca | 0 | 0 | 0 | 0 |

*Mismatch(es) with animal species are indicated in bold and underlined letters

### Table 4.

TABLE 4a

| | Seq | | % | % Difference |
|---|---|---|---|---|
| AON ID | ID No. | Sequence (5′-3′) | PS^{a} | vs PS-DNA^{c} |
| TOP1731 | 1 | PS-tcatgagtggcagctgcaatt | 100 | N.A |
| TOP1731-F3 | 73 | PS-tcatgagtggcagctgcaatt | 100 | +21 |
| TOP1731-DAP | 74 | PS-tcxtgxgtggcxgctgcxxtt | 100 | −1 |
| TOP1731-PO | 75 | PO-tcatgagtggcagctgcaatt | 0 | −77 |
| TAP1731-PO-DAP | 76 | PO-tcxtgxgtggcxgctgcxxtt | 0 | −66 |
| TOP1731-PO-OMe1 | 77 | PO-TCATGagtggcagctgCAATT | 0 | −168 |
| TOP1731-P²M-1 | 78 | tₛcₛaₛtₛgₛaₛgₛtₛgₛgₒcₒaₒgₛcₛtₛgₛcₛaₛaₛtₛt | 85 | +7 |
| TOP1731-P²M-2 | 79 | tₛcₛaₛtₛgₛaₛgₛtₒgₒgₒcₒaₒgₒcₒtₛgₛcₛaₛaₛtₛt | 65 | −55 |
| TOP1731-P²M-3 | 80 | tₛcₛaₛtₛgₛaₒgₒtₒgₒgₒcₒaₒgₒcₒtₒgₛcₛaₛaₛtₛt | 50 | −30 |
| TOP1731-P²M-3-DAP | 81 | tₛcₛxₛtₛgₛxₒgₒtₒgₒgₒcₒxₒgₒcₒtₒgₛcₛxₛxₛtₛt | 50 | −21 |
| TOP1731-P²M-4 | 82 | tₛcₛaₛtₛgₒaₒgₒtₒgₒgₒcₒaₒgₒcₒtₒgₒcₒaₛaₛtₛt | 35 | −64 |
| TOP1731-P²M-5 | 83 | tₛcₛaₛtₛgₒaₒgₒtₛgₛgₛcₛaₛgₛcₒtₒgₒcₛaₛaₛtₛt | 70 | +4 |
| TOP1731-P²M-6 | 84 | tₛcₛaₛtₛgₒaₒgₒtₒgₛgₛcₛaₛgₒcₒtₒgₒcₛaₛaₛtₛt | 60 | −29 |
| TOP1731-P²M-7 | 85 | tₛcₛaₒtₒgₛaₛgₒtₒgₛgₛcₛaₒgₒcₛtₛgₛcₒaₒaₛtₛt | 60 | +9 |
| TOP1731-P²M-7-DAP | 86 | tₛcₛxₒtₒgₛxₛgₒtₒgₛgₛcₛxₒgₒcₛtₛgₛcₒxₒxₛtₛt | 60 | +21 |
| TOP1731-P²M-8 | 87 | tₛcₛaₒtₒgₒaₒgₒtₒgₒgₒcₒaₒgₒcₒtₒgₒcₒaₛaₛtₛt | 25 | −79 |
| TOP1731-P²M-8-DAP | 88 | tₛcₛxₒtₒgₒxₒgₒtₒgₒgₒcₒxₒgₒcₒtₒgₒcₒxₛxₛtₛt | 25 | −40 |
| TOP1731-P²M-9 | 89 | tₛcₛaₒtₒgₒaₒgₛtₛgₒgₒcₒaₒgₛcₛtₒgₒcₒaₒaₛtₛt | 40 | −46 |
| TOP1731-P²M-9-DAP | 90 | tₛcₛxₒtₒgₒxₒgₛtₛgₒgₒcₒxₒgₛcₛtₒgₒcₒxₒxₛtₛt | 40 | −30 |
| TOP1731-P²M-10 | 91 | tₛcₛaₒtₒgₛaₛgₒtₒgₒgₛcₛaₒgₒcₒtₛgₛcₒaₒaₛtₛt | 50 | −21 |
| TOP1731-P²M-10-DAP | 92 | tₛcₛxₒtₒgₛxₛgₒtₒgₒgₛcₛxₒgₒcₒtₛgₛcₒxₒxₛtₛt | 50 | +8 |
| TOP1731-LNA1 | 93 | PS-tcatggtggcagctgcaatt | 100 | N.A |

TABLE 4b

| | Seq | | % | % Difference |
|---|---|---|---|---|
| AON ID | ID No. | Sequence (5′-3′) | PS^{a} | vs PS-DNA^{c} |
| TOP1769 | 33 | PS-acagtgcatggcctgagtaa | 100 | N.A |
| TOP1769-P²M-7 | 94 | aₛcₛaₒgₒtₛgₛcₒaₒtₛgₛgₛcₒcₒtₛgₛaₛgₒtₒaₛaₛc | 60 | +37 |
| TOP1769-P²M-7-DAP | 95 | xₛcₛxₒgₒtₛgₛcₒxₒtₛgₛgₛcₒcₒtₛgₛxₛgₒtₒxₛxₛc | 60 | +43 |
| TOP1769-P²M-9 | 96 | aₛcₛaₒgₒtₒgₒcₛaₛtₒgₒgₒcₒcₛtₛgₒaₒgₒtₒaₛaₛc | 67 | +2 |
| TOP1769-P²M-9-DAP | 97 | xₛcₛxₒgₒtₒgₒcₛxₛtₒgₒgₒcₒcₛtₛgₒxₒgₒtₒxₛxₛc | 67 | −9 |
| TOP1769-P²M-10 | 98 | aₛcₛaₒgₒtₛgₛcₒaₒtₒgₛgₛcₒcₒtₒgₛaₛgₒtₒaₛaₛc | 50 | +11 |
| TOP1769-P²M-10-DAP | 99 | xₛcₛxₒgₒtₛgₛcₒxₒtₒgₛgₛcₒcₒtₒgₛxₛgₒtₒxₛxₛc | 50 | +27 |

TABLE 4c

| | Seq | | % | % Difference |
|---|---|---|---|---|
| AON ID | ID No. | Sequence (5′-3′) | PS^{a} | vs PS-DNA^{c} |
| TOP1777 | 41 | PS-taagtaacagtgcatggcctg | 100 | N.A |
| TOP1777-P²M-7 | 100 | tₛaₛaₒgₒtₛaₛaₒcₒaₛgₛtₛgₒcₒaₛtₛgₛgₒcₒcₛtₛg | 60 | +13 |
| TOP1777-P²M-7-DAP | 101 | tₛxₛxₒgₒtₛxₛxₒcₒxₛgₛtₛgₒcₒxₛtₛgₛgₒcₒcₛtₛg | 60 | +29 |
| TOP1777-P²M-9 | 102 | tₛaₛaₒgₒtₒaₒaₛcₛaₒgₒtₒgₒcₛaₛtₒgₒgₒcₒcₛtₛg | 67 | −31 |
| TOP1777-P²M-9-DAP | 103 | tₛxₛxₒgₒtₒxₒxₛcₛxₒgₒtₒgₒcₛxₛtₒgₒgₒcₒcₛtₛg | 67 | −13 |
| TOP1777-P²M-10 | 104 | tₛaₛaₒgₒtₛaₛaₒcₒaₒgₛtₛgₒcₒaₒtₛgₛgₒcₒcₛtₛg | 50 | −47 |
| TOP1777-P²M-10-DAP | 105 | tₛxₛxₒgₒtₛxₛxₒcₒxₒgₛtₛgₒcₒxₒtₛgₛgₒcₒcₛtₛg | 50 | −8 |

TABLE 4d

| | | | | % Difference vs. | |
|---|---|---|---|---|---|
| | Seq | | | PS-DNA^{c} | |
| AON ID | ID No. | Sequence (5′-3′) | % PS^{a} | PDE4D | PDE4B |
| TOP1572 | 42 | PS-ggttgctcagitctgcaca | 100 | N.A | N.A |
| TOP1572-F2 | 106 | PS-ggttgctcagitctgcaca | 100 | +5 | +20 |
| TOP1572-DAP | 107 | PS-ggttgctcxgitctgcxcx | 100 | −1 | −3 |
| TOP1572-PO | 108 | PO-ggttgctcagitctgcaca | 0 | −58 | −113 |
| TOP1572-PO-DAP | 109 | PO-ggttgctcxgitctgcxcx | 0 | −24 | +3 |
| TOP1572-PO-OMe1 | 110 | PO-GGTTGctcagitctGCACA | 0 | −47 | −112 |
| TOP1572-P²M-1 | 112 | gₛgₛtₛtₛgₛcₛtₛcₛaₒgₒiₒtₛcₛtₛgₛcₛaₛcₛa | 83 | −4 | −4 |
| TOP1572-P²M-2 | 112 | gₛgₛtₛtₛgₛcₛtₒcₒaₒgₒiₒtₒcₒtₛgₛcₛaₛcₛa | 56 | −22 | −37 |
| TOP1572-P²M-3 | 113 | gₛgₛtₛtₛgₛcₒtₒcₒaₒgₒiₒtₒcₒtₒgₛcₛaₛcₛa | 50 | −53 | −25 |
| TOP1572-P²M-3-DAP | 114 | gₛgₛtₛtₛgₛcₒtₒcₒxₒgₒiₒtₒcₒtₒgₛcₛxₛcₛx | 50 | −20 | +9 |
| TOP1572-P²M-4 | 115 | gₛgₛtₛtₛgₒcₒtₒcₒaₒgₒiₒtₒcₒtₒgₒcₛaₛcₛa | 39 | +2 | −26 |
| TOP1572-P²M-5 | 116 | gₛgₛtₛtₛgₒcₒtₒcₛaₛgₛiₛtₛcₒtₒgₒcₛaₛcₛa | 67 | −16 | −36 |
| TOP1572-P²M-6 | 117 | gₛgₛtₛtₛgₒcₒtₒcₒaₛgₛiₛtₒcₒtₒgₒcₛaₛcₛa | 56 | −12 | −35 |
| TOP1572-P²M-7 | 118 | gₛgₛtₒtₒgₛcₛtₒcₒaₛgₛiₒtₒcₛtₛgₒcₒaₛcₛa | 56 | −9 | −1 |
| TOP1572-P²M-7-DAP | 119 | gₛgₛtₒtₒgₛcₛtₒcₒxₛgₛiₒtₒcₛtₛgₒcₒxₛcₛx | 56 | 0 | −3 |
| TOP1572-P²M-8 | 120 | gₛgₛtₒtₒgₒcₒtₒcₒaₒgₒiₒtₒcₒtₒgₒcₛaₛcₛa | 28 | −61 | −82 |
| TOP1572-P²M-8-DAP | 121 | gₛgₛtₒtₒgₒcₒtₒcₒxₒgₒiₒtₒcₒtₒgₒcₛxₛcₛx | 28 | −27 | −43 |
| TOP1572-P²M-9 | 122 | gₛgₛtₒtₒgₒcₛtₛcₒaₒgₒiₒtₛcₛtₒgₒcₒaₛcₛa | 44 | −32 | −36 |
| TOP1572-P²M-9-DAP | 123 | gₛgₛtₒtₒgₒcₛtₛcₒxₒgₒiₒtₛcₛtₒgₒcₒxₛcₛx | 44 | −19 | −16 |
| TOP1572-P²M-10 | 124 | gₛgₛtₛtₒgₒcₒtₒcₛaₛgₛiₛtₒcₒtₒgₒcₛaₛcₛa | 56 | −49 | −64 |
| TOP1572-P²M-10-DAP | 125 | gₛgₛtₛtₒgₒcₒtₒcₒxₛgₛiₛtₒcₒtₒgₒcₛxₛcₛx | 56 | −22 | −23 |
| TOP1572-P²M-11 | 126 | gₛgₛtₒtₒgₒcₛtₛcₒaₒgₒiₛtₛcₒtₒgₒcₛaₛcₛa | 50 | −35 | −52 |
| TOP1572-P²M-11-DAP | 127 | gₛgₛtₒtₒgₒcₛtₛcₒxₒgₒiₛtₛcₒtₒgₒcₛxₛcₛx | 50 | −20 | −30 |

| Sugar Chemistry | Symbol: |
|---|---|
| DNA | lower case |
| FANA | bold lowercase |
| 2′-OMe-RNA | BOLD UPPERCASE |
| LNA | Underlined italic lowercase |
| Linkage Chemistry | Symbol: |
| All Phosphodiester (PO) | Denote sequence with prefix PO- |
| All Phosphorothioate | Denote sequence with prefix PS- |
| (PS) | |
| Mixed PO/PS linkages | Denote PS with s subscript and PO with o subscript |
| (P²M) | |
| Base Chemistry | Symbol: |
| inosine | i |
| 2-amino-2′-deoxy- | x |
| adensine | |

^{a}Percentage of phosphorothioate linkage in AON sequence.
^{c}Percentage of difference compared to PDE mRNA inhibition in Flp-In T-Rex 293 cells treated with 236 nM PS-DNA.

TABLE 5

| | Seq | | |
|---|---|---|---|
| AON ID | ID No. | Sequence (5′-3′) | % PS^{a} |
| TOP1731s | 128 | PS-agtactcaccgtcgacgttaa | 100 |
| TOP1769s | 129 | PS-tgtcacgtaccggactcattg | 100 |
| TOP1777s | 130 | PS-attcattgtcacgtaccggac | 100 |
| TOP1572s | 131 | PS-ccaacgagtciagacgtgt | 100 |
| TOP1777s-P²M-7-DAP | 132 | xₛtₛtₒcₒxₛtₛtₒgₒtₛcₛxₛcₒgₒtₛxₛcₛcₒgₒgₛxₛc | 60 |
| TOP1731s-P²M-7-DAP | 155 | xₛgₛtₒxₒcₛtₛcₒxₒcₛcₛgₛtₒcₒgₛxₛcₛgₒtₒtₛxₛx | 60 |
| TOP1572s-P²M-7-DAP | 156 | cₛcₛxₒxₒcₛgₛxₒgₒtₛcₛiₒxₒgₛxₛcₒgₒtₛgₛt | 56 |
| CpG | 133 | gₛgₛgₒgₒgₒaₒgₒaₒtₒcₒgₒtₒcₒgₛgₛgₛgₛgₛg | 37 |

| Sugar Chemistry | Symbol: |
|---|---|
| DNA | lower case |
| Linkage Chemistry | Symbol: |
| All Phosphorothioate (PS) | Denote sequence with prefix PS- |
| Mixed PO/PS linkages (P²M) | Denote PS with s subscript and PO with o subscript |
| Base Chemistry | Symbol |
| inosine | i |
| 2-amino-2′-deoxyadenosine | x |

^{a}Percentage of phosphorothioate linkage in AON sequence.

TABLE 6

| | Seq | | |
|---|---|---|---|
| AON ID | ID No. | Sequence (5′-3′) | % PS^{a} |
| TOP27-7-F3 | 134 | PS-tccagatcgtgagtggc | 100 |
| TOP2707Mi2-F3 | 135 | PS-tgctcaacgtctgaggg | 100 |
| TOP2707-P²M-7-DAP | 136 | tₛcₛcₒxₒgₒxₛtₛcₒgₒtₒgₛxₛgₒtₒgₛgₛc | 50 |
| TOP2804-F1 | 137 | PS-ggttgctcaggtctgcacag | 100 |
| TOP2804-P²M-7-DAP | 138 | gₛgₛtₒtₒgₛcₛtₒcₒxₛgₛgₒtₒcₛtₛgₒcₒxₛcₛxₛg | 58 |

| Sugar Chemistry | Symbol: |
|---|---|
| DNA | lower case |
| FANA | bold lowercase |
| Linkage Chemistry | Symbol: |
| All Phosphodiester (PO) | Denote sequence with prefix PO- |
| All Phosphorothioate | Denote sequence with prefix PS- |
| (PS) | |
| Mixed PO/PS linkages | Denote PS with s subscript and PO with |
| (P²M) | o subscript |
| Base Chemistry | Symbol: |
| 2-amino-2′- | x |
| deoxyadenosine | |

^{a}Percentage of phosphorothioate linkage in AON sequence.

TABLE 7

| Target | Sense/Antisense | SEQ |
|---|---|---|
| gene | primer sequence (5′-3′) | ID No. |
| PDE7A | TCAGGCCATGCACTGTTACT | 139 |
| | CCTGATTCTCTCAATAAGCCC | 140 |
| PDE4B | TGGCAGACCTGAAGACAATG | 141 |
| | AAATTCCTCCATGATGCGG | 142 |
| PDE4D | CAGAATATGGTGCACTGTGC | 143 |
| | AGTCTATGAAGCCCACCTGTG | 144 |
| MCP-1 | CAATAGGAAGATCTCAGTGC | 145 |
| | GTGTTCAAGTCTTCGGAGTT | 146 |
| MMP-2 | AGGGCACATCCTATGACAGC | 147 |
| | ATTTGTTGCCCAGGAAAGTG | 148 |
| MMP-12 | CCAGTGACAAAAATGAAATATAGTGGAA | 149 |
| | GACTCCACATCGAGGTGCGT | 150 |
| IL-1β | CAGGGACAGGATATGGAGCAA | 151 |
| | GCAGACTCAAATTCCAGCTTGTTA | 152 |
| B2M | CAAGGACTGGTCTTTCTATCTCTTGT | 153 |
| | GTGGAGCAACCTGCTCAGATAC | 154 |

| | | | | TOP1572 | | TOP1731 | TOP1769- | TOP1777- |
|---|---|---|---|---|---|---|---|---|
| | | | TOP1572- | P²M-7 | | P²M-7- | P²M-7- | P²M-7- |
| | PBS | TOP1572 | P²M-7 | DAP | TOP1731 | DAP | DAP | DAP |
| Dose (mg/kg) | — | 2.5 | 2.5 | 2.5 | 2.5 | 2.5 | 2.5 | 2.5 |

BAL

| | | | | | | | | |
|---|---|---|---|---|---|---|---|---|
| Total cells | 2.7 × 10⁵ | 1.1 × 10⁶ | 1.6 × 10⁵ | 3.4 × 10⁵ | 4.0 × 10⁶ | 3.0 × 10⁵ | 3.1 × 10⁵ | 2.7 × 10⁵ |
| | (6.4 × 10⁴) | (2.7 × 10⁵) | (3.5 × 10⁴) | (7.9 × 10⁴) | (1.1 × 10⁶) | (5.5 × 10⁴) | (9.7 × 10⁴) | (1.7 × 10⁵) |
| Neutrophils | 4.3 × 10³ | 8.4 × 10⁴ | 5.7 × 10³ | 1.7 × 10⁴ | 8.9 × 10⁵ | 3.9 × 10³ | 2.2 × 10⁴ | 3.2 × 10³ |
| | (2.7 × 10³) | (6.9 × 10⁴) | (2.0 × 10³) | (5.3 × 10³) | (6.2 × 10⁵) | (2.6 × 10³) | (1.1 × 10⁴) | (2.6 × 10³) |
| Macrophage | 2.6 × 10⁵ | 9.1 × 10⁵ | 1.4 × 10⁵ | 3.1 × 10⁵ | 2.4 × 10⁶ | 3.0 × 10⁵ | 3.0 × 10⁵ | 1.3 × 10⁵ |
| | (5.8 × 10⁴) | (3.1 × 10⁵) | (3.6 × 10⁴) | (8.1 × 10⁴) | (4.7 × 10⁵) | (7.0 × 10⁴) | (8.7 × 10⁴) | (2.4 × 10⁴) |
| Lymphocytes | 8.1 × 10³ | 1.9 × 10⁵ | 1.1 × 10⁴ | 1.1 × 10⁴ | 6.8 × 10⁵ | 9.4 × 10³ | 2.0 × 10⁴ | 3.2 × 10⁴ |
| | (1.1 × 10⁴) | (5.4 × 10⁴) | (3.2 × 10³) | (5.0 × 10³) | (2.5 × 10⁵) | (3.8 × 10³) | (1.5 × 10⁴) | (1.4 × 10⁴) |
| % Large | 6.0 | 61.0 | 33.0 | 18.0 | 55.0 | 37.5 | 18.2 | 15.7 |
| macrophages | (1.8) | (4.3) | (8.5) | (4.4) | (4.1) | (5.2) | (5.4) | (5.9) |

LUNG
Infiltrate, mixed cell, perivascular

| | | | | | | | | |
|---|---|---|---|---|---|---|---|---|
| Grade 1 | — | 1/4 | — | — | 1/4 | — | — | — |
| Grade 2 | — | 1/4 | — | — | 1/4 | — | — | — |
| Grade 3 | — | 2/4 | — | — | 2/4 | — | — | — |

Inflammation, alveolar (chronic active)

| | | | | | | | | |
|---|---|---|---|---|---|---|---|---|
| Grade 1 | — | — | — | — | | 1/4 | — | — |
| Grade 2 | — | 1/4 | — | — | 1/4 | — | — | — |
| Grade 3 | — | 1/4 | — | — | 3/4 | — | — | — |
| Grade 4 | — | 2/4 | — | — | — | — | — | — |

Infiltrate, macrophages alveolar

| | | | | | | | | |
|---|---|---|---|---|---|---|---|---|
| Grade 1 | — | — | — | 4/4 | — | 3/4 | 1/4 | 2/4 |
| Grade 2 | — | — | — | — | 4/4 | — | 3/4 | — |

Lung Toxicity Index*

| | | | | | | | |
|---|---|---|---|---|---|---|---|
| — | 5.5 | 0 | 1 | 7 | 1 | 1.75 | 0.5 |

Efficacy Index**

| | | | | | | | |
|---|---|---|---|---|---|---|---|
| — | 75%^{A} | 50%^{A} | 71%^{A} | 70% | 91% | 89% | 66% |

Efficacy/Toxicity Ratio***

| | | | | | | | |
|---|---|---|---|---|---|---|---|
| — | 0.14 | >10 | 0.71 | 0.1 | 0.91 | 0.51 | 1.32 |

BAL: values represent mean (± SEM)
LUNG: values represent number of animals with finding/total animals
A: mean efficacy for the two targets (PDE4B and PDE4D)
*Lung Toxicity Index = Σ(finding % *Severity Grade)
For example for TOP1572:
Lung Toxicity Index = (0.25*1 + 0.25*2 + 0.5*3) + (0.25*2 + 0.25*3+300.5*4) = 5.5
**Efficacy Index = % maximal target knockdown in vitro
***Efficacy/Toxicity Ratio = Efficacy Index/Lung Toxicity Index

TABLE 9

| | Seq | | |
|---|---|---|---|
| AON ID | ID No. | Sequence (5′-3′) | % PS^{a} |
| TOP004 | 157 | PS-gggtctgcxgcgggxtggt | 100 |
| TOP005 | 158 | PS-gttxctxcttccxcctgcctg | 100 |
| TOP006 | 159 | PS-tggcactttaggtggctg | 100 |
| TOP007 | 160 | PS-actcatattcatagggtg | 100 |

| Sugar Chemistry | Symbol: |
|---|---|
| DNA | lower case |
| Linkage Chemistry | Symbol: |
| All Phosphoro- | Denote sequence with prefix PS- |
| thioate (PS) | |
| Base Chemistry | Symbol: |
| 2-amino-2′-deoxy- | x |
| adenosine | |

^{a}Percentage of phosphorothioate linkage in AON sequence.

### Table 10

TABLE 10a

| | Seq | | % | % Difference |
|---|---|---|---|---|
| AON ID | ID No. | Sequence (5′-3′) | PS^{a} | vs PS-DNA^{c} |
| TOP030 | 161 | PS-cacctctgtcaccagcatg | 100 | N.A |
| TOP030-P²M-7-DAP | 162 | cₛxₛcₒcₒtₛcₛtₒgₒtₛcₛxₒcₒcₛxₛgₒcₒxₛtₛg | 56 | −47 |
| TOP030-P²M-8-DAP | 163 | cₛxₛcₛcₒtₒcₛtₛgₒtₒcₒxₒcₛcₛxₒgₒcₛxₛtₛg | 56 | −14 |
| TOP030-P²M-9-DAP | 164 | cₛxₛcₒcₒtₒcₛtₛgₒtₒcₒxₛcₛcₒxₒgₒcₛxₛtₛg | 50 | −16 |
| TOP030-P²M-10-DAP | 165 | cₛxₛcₒcₒtₒcₛtₛgₒtₒcₒxₛcₛcₒxₒgₒcₒxₛtₛg | 50 | −35 |
| TOP030-P²M-11-DAP | 166 | cₛxₛcₛcₒtₛcₛtₒgₛtₛcₛxₒcₛcₛxₛgₛcₒxₛtₛg | 78 | −4 |
| TOP030-P²M-12-DAP | 167 | cₛxₛcₒcₒtₛcₛtₛgₛtₒcₒxₛcₛcₛxₒgₒcₛxₛtₛg | 67 | −33 |
| TOP030-P²M-13-DAP | 168 | cₛxₛcₛcₛtₛcₛtₒgₒtₒcₒxₒcₒcₛxₛgₛcₛxₛtₛg | 67 | −25 |
| TOP030-P²M-14-DAP | 169 | cₛxₛcₛcₛtₛcₛtₛgₒtₒcₒxₛcₛcₛxₛgₛcₛxₛtₛg | 78 | −51 |
| TOP030-P²M-15-DAP | 170 | cₛxₛcₒcₒtₒcₛtₛgₛtₛcₛxₛcₛcₒxₒgₒcₛxₛtₛg | 67 | −11 |
| TOP031 | 171 | PS-gtacttccggaacctctctcc | 100 | N.A |
| TOP031-P²M-1-DAP | 172 | gₛtₛxₒcₒtₛtₛcₒcₒgₛgₛxₒxₒcₛcₛtₒcₒtₛcₛtₛcₛc | 60 | +2 |
| TOP031-P²M-2-DAP | 173 | gₛtₛxₛcₒtₛtₛcₒcₒgₒgₒxₒxₛcₛcₛtₛcₒtₒcₒtₛcₛc | 60 | −2 |
| TOP031-P²M-3-DAP | 174 | gₛtₛxₒcₒtₒtₛcₛcₛgₛgₛxₛxₛcₛcₛtₛcₒtₒcₒtₛcₛc | 70 | +9 |
| TOP031-P²M-4-DAP | 175 | gₛtₛxₛcₒtₛtₛcₛcₒgₛgₛxₛxₒcₛcₛtₛcₒtₛcₛtₛcₛc | 80 | +14 |
| TOP031-P²M-5-DAP | 176 | gₛtₛxₛcₒtₒtₛcₛcₛgₛgₒxₒxₛcₛcₛtₛcₛtₒcₒtₛcₛc | 70 | +6 |
| TOP031-P²M-6-DAP | 177 | gₛtₛxₛcₛtₛtₛcₛcₒgₒgₒxₒxₒcₒcₛtₛcₛtₛcₛtₛcₛc | 70 | −1 |
| TOP031-P²M-7-DAP | 178 | gₛtₛxₛcₛtₛtₛcₛcₛgₒgₒxₒxₒcₛcₛtₛcₛtₛcₛtₛcₛc | 80 | −1 |

| Sugar Chemistry | Symbol: |
|---|---|
| DNA | lower case |
| FANA | bold lowercase |
| Linkage Chemistry | Symbol: |
| All Phosphorothioate | Denote sequence with prefix PS- |
| (PS) | |
| Mixed PO/PS linkages | Denote PS with s subscript and PO with o subscript |
| (P²M) | |
| Base Chemistry | Symbol: |
| 2-amino-2′-deoxyadenosine | x |

^{a}Percentage of phosphorothioate linkage in AON sequence.

TABLE 10b

| | Seq | | % | % Difference |
|---|---|---|---|---|
| AON ID | ID No. | Sequence (5′-3′) | PS^{a} | vs PS-DNA^{c} |
| TOP062 | 179 | PS-ctctccacttccacggcctg | 100 | N.A |
| TOP062-P²M-7-DAP | 180 | cₛtₛcₒtₒcₛcₛxₒcₒtₛtₛcₒcₒxₛcₛgₒgₒcₛcₛtₛg | 58 | −29 |
| TOP062-P²M-8-DAP | 181 | cₛtₛcₒtₒcₛcₛxₒcₒtₛtₛcₒcₒxₒcₛgₛgₒcₒcₛtₛg | 53 | −41 |
| TOP062-P²M-9-DAP | 182 | cₛtₛcₛtₒcₒcₒxₛcₛtₒtₒcₒcₛxₛcₒgₒgₒcₛcₛtₛg | 53 | −26 |
| TOP062-P²M-10-DAP | 183 | cₛtₛcₒtₒcₒcₛxₛcₒtₒtₒcₒcₒxₛcₛgₒgₒcₒcₛtₛg | 42 | −27 |
| TOP062-P²M-11-DAP | 184 | cₛtₛcₛtₒcₛcₛxₛcₒtₛtₛcₛcₒxₛcₛgₛgₒcₛcₛtₛg | 79 | +2 |
| TOP062-P²M-12-DAP | 185 | cₛtₛcₒtₒcₛcₛxₛcₛtₒtₒcₛcₛxₛcₛgₒgₒcₛcₛtₛg | 68 | −15 |
| TOP062-P²M-13-DAP | 186 | cₛtₛcₛtₛcₛcₛxₛcₒtₒtₒcₒcₒxₒcₛgₛgₛcₛcₛtₛg | 68 | −20 |
| TOP062-P²M-14-DAP | 187 | cₛtₛcₛtₛcₛcₛxₛcₛtₒtₒcₒcₒxₛcₛgₛgₛcₛcₛtₛg | 79 | −15 |
| TOP062-P²M-15-DAP | 188 | cₛtₛcₒtₒcₒcₛxₛcₛtₛtₛcₛcₛxₛcₒgₒgₒcₛcₛtₛg | 68 | −8 |
| TOP057 | 189 | PS-gaccgagctggccacctcc | 100 | N.A |
| TOP057-P²M-1DAP | 190 | gₛxₛcₒcₒgₛxₛgₒcₒtₛgₛgₒcₒcₛxₛcₒcₒtₛcₛc | 56 | −34 |
| TOP057-P²M-2-DAP | 191 | gₛxₛcₒcₒgₒxₛgₛcₒtₒgₒgₒcₛcₛxₛcₒcₒtₛcₛc | 56 | −32 |
| TOP057-P²M-3-DAP | 192 | gₛxₛcₒcₒgₒxₛgₛcₛtₛgₛgₛcₛcₛxₒcₒcₒtₛcₛc | 67 | −4 |
| TOP057-P²M-4-DAP | 193 | gₛxₛcₛcₒgₛxₛgₛcₒtₛgₛgₛcₒcₛxₛcₛcₒtₛcₛc | 78 | +5 |
| TOP057-P²M-5-DAP | 194 | gₛxₛcₛcₒgₒxₛgₛcₛtₒgₒgₛcₛcₛxₛcₒcₒtₛcₛc | 67 | −16 |
| TOP057-P²M-6-DAP | 195 | gₛxₛcₛcₛgₛxₛgₒcₒtₒgₒgₒcₒcₛxₛcₛcₛtₛcₛc | 67 | −3 |
| TOP057-P²M-7-DAP | 197 | gₛxₛcₛcₛgₛxₛgₛcₒtₒgₒgₒcₛcₛxₛcₛcₛtₛcₛc | 78 | +3 |

| Sugar Chemistry | Symbol: |
|---|---|
| DNA | lower case |
| FANA | bold lowercase |
| Linkage Chemistry | Symbol: |
| All Phosphorothioate | Denote sequence with prefix PS- |
| (PS) | |
| Mixed PO/PS linkages | Denote PS with s subscript and PO with o subscript |
| (P²M) | |
| Base Chemistry | Symbol: |
| 2-amino-2′-deoxyadenosine | x |

^{a}Percentage of phosphorothioate linkage in AON sequence.

1. An oligonucleotide directed against a target gene, wherein the oligonucleotide is capable of hybridizing to at least a portion of a nucleic acid sequence encoding the gene under stringent conditions, and wherein,
at least one nucleotide of the oligonucleotide is 2-amino-2′-deoxyadenosine (DAP); and
the internucleoside linkages of the oligonucleotide comprises at least three alternating segments, each segment consisting of either at least one phosphorothioate or at least one phosphodiester bond.

2. The oligonucleotide of claim 1, wherein the target gene is the common beta sub-unit of IL-3, IL-5 and GM-CSF.

3. The oligonucleotide of claim 1, wherein the target gene is CCR3 chemokine receptor.

4. The oligonucleotide of claim 1, wherein the target gene is a target Phosphodiesterase (PDE).

5. The oligonucleotide of claim 4, wherein the target PDE is selected from the group consisting of PDE3A, PDE3B, PDE4A, PDE4B, PDE4C, PDE4D, PDE7A1, PDE7A2, PDE7A3 and PDE7B.

6. The oligonucleotide of claim 5, wherein the target PDE is PDE7A.

7. The oligonucleotide of claim 5, wherein the target PDE is PDE4B.

8. The oligonucleotide of claim 5, wherein the target PDE is PDE4D.

9. The oligonucleotide of claim 1, wherein the ratio of phosphorothioate to phosphodiester bonds is between 30:70 and 70:30.

10. The oligonucleotide of claim 9, wherein the ratio of phosphorothioate to phosphodiester bonds is between 40:60 and 60:40.

11. The oligonucleotide of claim 10, wherein the ratio of phosphorothioate to phosphodiester bonds is between 45:55 and 55:45.

12. The oligonucleotide of claim 11, wherein the ratio of phosphorothioate to phosphodiester bonds is about 50:50.

13. The oligonucleotide of claim 1, wherein the oligonucleotide is at least 80% complementary to the target gene.

14. The oligonucleotide of claim 13, wherein the oligonucleotide is 100% complementary to the target gene.

15. The oligonucleotide of claim 1, wherein the efficacy/toxicity ratio is greater than 0.25.

16. The oligonucleotide of claim 1, wherein the oligonucleotide is between 15-25 nucleotides in length.

17. The oligonucleotide of claim 16, wherein the oligonucleotide is between 18-22 nucleotides in length.

18. The oligonucleotide of claim 4, having a base sequence selected from the group consisting of SEQ ID NOs. 1-72, preferably SEQ ID NOs. 1, 4, 8, 33, 36, 41 and 42, wherein at least one adenosine is replaced with DAP.

19. The oligonucleotide of claim 18, wherein the oligonucleotide has a base sequence selected from the group consisting of SEQ ID NOs. 1, 33, 41 and 42.

20. The oligonucleotide of claim 4, comprising any one of SEQ ID NOs. 81, 86, 90, 92, 95, 97, 99, 101, 103, 105, 114, 119, 121, 123, 125 and 127, preferably SEQ ID NOs. 86, 95, 101 and 119.

21. The oligonucleotide of claim 20, consisting of any one of SEQ ID NOs. 81, 86, 90, 92, 95, 97, 99, 101, 103, 105, 114, 119, 121, 123, 125 and 127, preferably SEQ ID NOs. 86, 95, 101 and 119.

22. The oligonucleotide of claim 2, having a base sequence selected from the group consisting of SEQ ID NOs. 179 and 189, wherein at least one adenosine is replaced with DAP.

23. The oligonucleotide of claim 22, comprising any one of SEQ ID NOs. 180-188 and 190-197.

24. The oligonucleotide of claim 3, having a base sequence selected from the group consisting of SEQ ID NOs. 161 and 171, wherein at least one adenosine is replaced with DAP.

25. The oligonucleotide of claim 24, comprising any one of SEQ ID NOs. 162-170 and 172-178.

26. A pharmaceutical composition comprising the oligonucleotide of claim 1 and a pharmaceutically acceptable carrier.

27. The pharmaceutical composition of claim 26 comprising at least two oligonucleotides of claim 1.

28. The pharmaceutical composition of claim 27, wherein the at least two oligonucleotides are directed against PDE7A and PDE4B respectively.

29. The pharmaceutical composition of claim 27, wherein the at least two oligonucleotides are directed against PDE7A and PDE4D respectively.

30. The pharmaceutical composition of 28, wherein the oligonucleotide directed against PDE4B or PDE4D also downregulates PDE4D.

31. The pharmaceutical composition of claim 27, wherein the at least two oligonucleotides are directed against the common beta sub-unit of IL-3, IL-5 and GM-CSF, and CCR3 chemokine receptor respectively.

32-34. (canceled)

35. A method of treating respiratory disease in a subject comprising administering the pharmaceutical composition of claim 26.

36-37. (canceled)

38. An oligonucleotide comprising the base sequence of any one of SEQ ID NOs. 1-72, 161, 171, 179 and 189, preferably SEQ ID NOs. 1, 4, 8, 33, 36, 41, 42, 161, 171, 179 and 189.

39. The oligonucleotide of claim 38, wherein at least one adenosine is replaced with DAP.

40. An oligonucleotide consisting of any one of SEQ ID NOs. 1-127 and 161-197.

41. The method of claim 35, wherein the respiratory disease is at least one of chronic obstructive pulmonary disease, asthma, eosinophilic cough, bronchitis, acute and chronic rejection of lung allograft, sarcoidosis, pulmonary fibrosis, rhinitis, sinusitis, viral infection or a neoplastic disease.
